SEQUENCE LISTING (15 sequences)

SEQ ID NO: 1 - protein, 100 aa, Human immunodeficiency virus type 1
VEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLN

SEQ ID NO: 2 - protein, 39 aa, Human immunodeficiency virus type 1
GGGDMRDNWRSELYKYKVVKIEPLGVAPTKAKRRVVQRE

SEQ ID NO: 3 - protein, 12 aa, Human immunodeficiency virus type 1
KRRVVQREKRAV

SEQ ID NO: 4 - protein, 15 aa, Human immunodeficiency virus type 1
APTKAKRRVVQREKR

SEQ ID NO: 5 - DNA, 2037 bp, Artificial Sequence
  Derived from HIV-1
atggatgcaatgaagagagggctctgctgtgtgctgctgctgtgtggagcagtcttcgtttcgcccagccaggaaatccatgcccgattcagaagaggcgccgctgtggagaagctgtgggtgactgtatactatggggtgcctgtgtggaaggaggccaccaccaccctgttctgtgcctctgatgccaaggcctatgacactgaggtccacaatgtctgggccacccatgcctgtgtgcccactgaccccaaccctcaggaggtggtgctggagaatgtgactgagcacttcaacatgtggaagaacaacatggtggagcagatgcaggaggacatcatcagcctgtgggaccagagcctgaagccctgtgtgaagctgacccccctgtgtgtgaccctgaactgcaaggatgtgaatgccaccaacaccaccaatgactctgagggcactatggagaggggtgagatcaagaactgcagcttcaacatcaccaccagcatcagggatgaggtgcagaaggagtatgccctgttctacaagctggatgtggtgcccattgacaacaacaacaccagctacaggctgatcagctgtgacacctctgtgatcacccaggcctgccccaagatcagctttgagcccatccccatccactactgtgcccctgctggctttgccatcctgaagtgcaatgacaagaccttcaatggcaaaggcccttgcaagaatgtgagcactgtgcagtgcactcatggcatcaggcctgtggtgagcacccagctgctgctgaatggcagcctggctgaggaggaggtggtgatcaggtctgacaacttcaccaacaatgccaagaccatcattgtgcagctgaaggagtctgtggagatcaactgcaccaggcccaacaacaacaccaggaagagcattcacattggccctggcagggccttctacaccactggggagatcattggggacatcaggcaggcccactgcaacatcagcagggccaagtggaatgacaccctgaagcagattgtgatcaagctgagggagcagtttgagaacaagaccattgtgttcaatcacagctctggtggtgatcctgagattgtgatgcacagcttcaactgtggtggtgagttcttctactgcaacagcacccagctgttcaacagcacctggaacaacaacactgagggcagcaacaacactgagggcaacaccatcaccctgccttgcaggatcaagcagatcatcaacatgtggcaggaggtgggcaaggccatgtatgctcctcccatcaggggccagatcaggtgcagcagcaacatcactggcctgctgctgaccagggatggtggcatcaatgagaatggcactgagattttcaggcctggtggtggggacatgagggacaactggaggtctgagctgtacaagtacaaggtggtgaagattgagccccttggtgtggctcccaccaaggctaagcgcagggtggtgcagagggagaagcgcgctgtgggcattggggctgtgttcctgggcttccttggggctgctggcagcactatgggtgctgccagcatgaccctgactgtgcaggccaggctgctgctgtctggcattgtgcagcagcagaacaacctgctgagggccattgaggctcaacagaggatgctccagctcactgtctggggcatcaagcagctccaggccagggtgctggctgtggagaggtatcttggggatcagcagctccttggcatctggggctgctctggcaagctgatctgcaccactgctgtgccctggaatgccagctggagcaacaagagcctggacaggatctggaacaacatgacctggatggagtgggagagggagattgacaactacacctctgagatttacaccctgattgaggagagccagaaccagcaggagaagaatgagcaggagctgctggagctggacaagtgggccagcctgtggaactggtttgacatcaccaagtggctgtggtactaa

SEQ ID NO: 6 - protein, 502 aa, Artificial Sequence
  Derived from HIV-1
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKR

SEQ ID NO: 7 - protein, 170 aa, Artificial Sequence
  Derived from HIV-1
AVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWY

SEQ ID NO: 8 - DNA, 2043 bp, Artificial Sequence
  Derived from HIV-1
atggatgcaatgaagagagggctctgctgtgtgctgctgctgtgtggagcagtcttcgtttcgcccagccaggaaatccatgcccgattcagaagaggcgccgctgtggagaagctgtgggtgactgtatactatggggtgcctgtgtggaaggaggccaccaccaccctgttctgtgcctctgatgccaaggcctatgacactgaggtccacaatgtctgggccacccatgcctgtgtgcccactgaccccaaccctcaggaggtggtgctggagaatgtgactgagcacttcaacatgtggaagaacaacatggtggagcagatgcaggaggacatcatcagcctgtgggaccagagcctgaagccctgtgtgaagctgacccccctgtgtgtgaccctgaactgcaaggatgtgaatgccaccaacaccaccaatgactctgagggcactatggagaggggtgagatcaagaactgcagcttcaacatcaccaccagcatcagggatgaggtgcagaaggagtatgccctgttctacaagctggatgtggtgcccattgacaacaacaacaccagctacaggctgatcagctgtgacacctctgtgatcacccaggcctgccccaagatcagctttgagcccatccccatccactactgtgcccctgctggctttgccatcctgaagtgcaatgacaagaccttcaatggcaaaggcccttgcaagaatgtgagcactgtgcagtgcactcatggcatcaggcctgtggtgagcacccagctgctgctgaatggcagcctggctgaggaggaggtggtgatcaggtctgacaacttcaccaacaatgccaagaccatcattgtgcagctgaaggagtctgtggagatcaactgcaccaggcccaacaacaacaccaggaagagcattcacattggccctggcagggccttctacaccactggggagatcattggggacatcaggcaggcccactgcaacatcagcagggccaagtggaatgacaccctgaagcagattgtgatcaagctgagggagcagtttgagaacaagaccattgtgttcaatcacagctctggtggtgatcctgagattgtgatgcacagcttcaactgtggtggtgagttcttctactgcaacagcacccagctgttcaacagcacctggaacaacaacactgagggcagcaacaacactgagggcaacaccatcaccctgccttgcaggatcaagcagatcatcaacatgtggcaggaggtgggcaaggccatgtatgctcctcccatcaggggccagatcaggtgcagcagcaacatcactggcctgctgctgaccagggatggtggcatcaatgagaatggcactgagattttcaggcctggtggtggggacatgagggacaactggaggtctgagctgtacaagtacaaggtggtgaagattgagccccttggtgtggctcccaccaagtgcaagcgcagggtggtgcagaggaggaggaggcgcagggctgtgggcattggggctgtgttcctgggcttccttggggctgctggcagcactatgggtgctgccagcatgaccctgactgtgcaggccaggctgctgctgtctggcattgtgcagcagcagaacaacctgctgagggcccccgaggctcaacagaggatgctccagctcactgtctggggcatcaagcagctccaggccagggtgctggctgtggagaggtatcttggggatcagcagctccttggcatctggggctgctctggcaagctgatctgctgcactgctgtgccctggaatgccagctggagcaacaagagcctggacaggatctggaacaacatgacctggatggagtgggagagggagattgacaactacacctctgagatttacaccctgattgaggagagccagaaccagcaggagaagaatgagcaggagctgctggagctggacaagtgggccagcctgtggaactggtttgacatcaccaagtggctgtggtactaa

SEQ ID NO: 9 - protein, 504 aa, Artificial Sequence
  Derived from HIV-1
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKCKRRVVQRRRRRR

SEQ ID NO: 10 - protein, 170 aa, Artificial Sequence
  Derived from HIV-1
AVGIGAVFLGFLGAAGSTMGAASMTLTVQARLLLSGIVQQQNNLLRAPEAQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELDKWASLWNWFDITKWLWY

SEQ ID NO: 11 - DNA, 2124 bp, Artificial Sequence
  Derived from HIV-1
atggatgcaatgaagagagggctctgctgtgtgctgctgctgtgtggagcagtcttcgtttcgcccagccaggaaatccatgcccgattcagaagaggcgccgctgtggagaagctgtgggtgactgtatactatggggtgcctgtgtggaaggaggccaccaccaccctgttctgtgcctctgatgccaaggcctatgacactgaggtccacaatgtctgggccacccatgcctgtgtgcccactgaccccaaccctcaggaggtggtgctggagaatgtgactgagcacttcaacatgtggaagaacaacatggtggagcagatgcaggaggacatcatcagcctgtgggaccagagcctgaagccctgtgtgaagctgacccccctgtgtgtgaccctgaactgcaaggatgtgaatgccaccaacaccaccaatgactctgagggcactatggagaggggtgagatcaagaactgcagcttcaacatcaccaccagcatcagggatgaggtgcagaaggagtatgccctgttctacaagctggatgtggtgcccattgacaacaacaacaccagctacaggctgatcagctgtgacacctctgtgatcacccaggcctgccccaagatcagctttgagcccatccccatccactactgtgcccctgctggctttgccatcctgaagtgcaatgacaagaccttcaatggcaaaggcccttgcaagaatgtgagcactgtgcagtgcactcatggcatcaggcctgtggtgagcacccagctgctgctgaatggcagcctggctgaggaggaggtggtgatcaggtctgacaacttcaccaacaatgccaagaccatcattgtgcagctgaaggagtctgtggagatcaactgcaccaggcccaacaacaacaccaggaagagcattcacattggccctggcagggccttctacaccactggggagatcattggggacatcaggcaggcccactgcaacatcagcagggccaagtggaatgacaccctgaagcagattgtgatcaagctgagggagcagtttgagaacaagaccattgtgttcaatcacagctctggtggtgatcctgagattgtgatgcacagcttcaactgtggtggtgagttcttctactgcaacagcacccagctgttcaacagcacctggaacaacaacactgagggcagcaacaacactgagggcaacaccatcaccctgccttgcaggatcaagcagatcatcaacatgtggcaggaggtgggcaaggccatgtatgctcctcccatcaggggccagatcaggtgcagcagcaacatcactggcctgctgctgaccagggatggtggcatcaatgagaatggcactgagattttcaggcctggtggtggggacatgagggacaactggaggtctgagctgtacaagtacaaggtggtgaagattgagccccttggtgtggctcccaccaagtgcaagcgcagggtggtgcagaggaggaggaggcgcagggctgtgggcattggggctgtgttcctgggcttccttggggctgctggcagcactatgggtgctgccagcatgaccctgactgtgcaggccaggctgctgctgtctggcattgtgcagcagcagaacaacctgctgagggccattgaggctcaacagaggatgctccagctcactgtctggggcatcaagcagctccaggccagggtgctggctgtggagaggtatcttggggatcagcagctccttggcatctggggctgctctggcaagctgatctgctgcactgctgtgccctggaatgccagctggagcaacaagagcctggacaggatctggaacaacatgacctggatggagtgggagagggagattgacaactacacctctgagatttacaccctgattgaggagagccagaaccagcaggagaagaatgagcaggagctgctggagctggacaagtgggccagcctgtggaactggtttgacatcaccaagtggctgtggtacatcaagattttcatcatgattgttggtggcctggtgggcctgaggctggtgttcactgtcctgagcattgtgaacagggtgtaa

SEQ ID NO: 12 - protein, 504 aa, Artificial Sequence; identical to SEQ ID NO: 9
  Derived from HIV-1

SEQ ID NO: 13 - protein, 197 aa, Artificial Sequence
  Derived from HIV-1
AVGIGAVFLGFLGAAGSTMGAASMTLTVQARLLLSGIVQQQNNLLRAIEAQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRV

SEQ ID NO: 14 - protein, 34 aa, Artificial Sequence
  Derived from HIV-1
TRPNNNTRKSIHIGPGRAFYTTGEIIGDIRQAHC

SEQ ID NO: 15 - protein, 36 aa, Artificial Sequence
  Derived from HIV-1
CTRPNNNTRKRIRIQRGPGRAFVTIGKIGNMRQAHC

(19) United States
(12) Patent Application Publication
(10) Pub. No.: US 2008/0274134 A1
(43) Pub. Date: Nov. 6, 2008

(54) Hiv-1 Neutralizing Antibodies Elicited By Trimeric Hiv-1 Envelope Glycoprotein Complex

(76) Inventors: Norbert Schulke, Dedham, MA (US); William C. Olson, Ossining, NY (US); Paul J. Maddon, Scarsdale, NY (US); John P. Moore, New York, NY (US); Simon Beddows, Merseyside (GB)

Correspondence Address: COOPER & DUNHAM, LLP, 1185 AVENUE OF THE AMERICAS, NEW YORK, NY, 10036, US

(21) Appl. No.: 11/629,843

(22) PCT Filed: Jun. 15, 2005
(86) PCT No.: PCT/US05/21091

§ 371 Date: Feb. 28, 2008

Related U.S. Application Data
(60) Provisional application No. 60/580,229, filed on Jun. 15, 2004.
(60) Provisional application No. 60/588,590, filed on Jul. 16, 2004.
(60) Provisional application No. 60/605,662, filed on Aug. 30, 2004.
(60) Provisional application No. 60/670,937, filed on Apr. 12, 2005.

(51) Int. Cl. A61K 39/00

(52) U.S. Cl. 424/196.11

(57) ABSTRACT

This invention provides methods for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of (a) a trimeric HIV-1 gp140 protein complex, or (b) a superparamagnetic particle and a trimeric HIV-1 gp140 protein complex so affixed thereto as to permit recognition of the complex by a subject's immune system. The invention also provides methods for prophylactically or therapeutically treating HIV-1 infection in a subject. The invention further provides uses of a nucleic acid prime and a protein boost composition for the manufacture of separate coadministerable medicaments to be administered to a subject.

This application claims the benefit of U.S. Provisional Application Nos. 60/580,229, filed Jun. 15, 2004; 60/588,590, filed Jul. 16, 2004; 60/605,662, filed Aug. 30, 2004; and 60/670,937, filed Apr. 12, 2005, the contents of which are hereby incorporated by reference into this application.

The invention disclosed herein was made with United States Government support under NIH contract number N01 AI 30030 and NIH grants AI36082 and AI 45463 from the National Institute of Allergy and Infectious Diseases (NIAID). Accordingly, the United States Government has certain rights in this invention.

Throughout this application, various publications are referenced in parentheses by author name and date. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

The present invention relates to the use of trimeric gp140 as an immunogen to generate antibodies which exhibit (1) high reactivity against the trimeric protein, and (2) significant neutralization activity against viral particles pseudotyped with envelopes from selected primary HIV-1 isolates and immortalized T cell line-adapted (TCLA) strains.

### BACKGROUND OF THE INVENTION

### HIV-1 Envelope Glycoproteins and Mediation of Cell Fusion

The human immunodeficiency virus type 1 (HIV-1) is the agent that causes Acquired Immunodeficiency Syndrome (AIDS), a lethal disease characterized by deterioration of the immune system. The initial phase of the HIV-1 replicative cycle involves the attachment of the virus to susceptible host cells followed by fusion of viral and cellular membranes.

These events are mediated by the exterior viral envelope glycoproteins which are first synthesized as a fusion-incompetent precursor envelope glycoprotein (Env) known as gp160. The gp160 glycoprotein is endo-proteolytically processed to the mature envelope glycoproteins gp120 and gp41, which are noncovalently associated with each other in a complex on the surface of the virus. The gp120 surface protein contains the high affinity binding site for human CD4, the primary receptor for HIV, as well as domains that interact with fusion coreceptors, such as the chemokine receptors CCR5 and CXCR4. The gp41 protein spans the viral membrane and contains at its amino-terminus a sequence of amino acids important for the fusion of viral and cellular membranes.

The native, fusion-competent form of the HIV-1 envelope glycoprotein complex is a trimeric structure composed of three gp120 and three gp41 subunits. The receptor-binding (CD4 and co-receptor) sites are located in the gp120 moieties, whereas the fusion peptides are located in the gp41 components (Chan et al., 1997; Kwong et al., 1998; Kwong et al., 2000; Poignard et al., 2001; Tan et al., 1997; Weissenhorn et al., 1997; Wyatt et al., 1998).

In the generally accepted model of HIV-1 fusion, the sequential binding of gp120 to CD4 and a coreceptor induces a series of conformational changes in the gp41 subunits, leading to the insertion of the fusion peptides into the host cell membrane in a highly dynamic process (Doms et al., 2000; Jones et al., 1998; Melikyan et al., 2000; Sattentau et al., 1991; Sullivan et al., 1998; Trkola et al., 1996; Wu et al., 1996; Wyatt and Sodroski, 1998; Zhang et al., 1999). The associations between the six components of the fusion-competent complex are maintained via non-covalent interactions between gp120 and gp41, and between the gp41 subunits (Poignard et al., 2001; Wyatt and Sodroski, 1998). These interactions are relatively weak, making the fusion-competent complex unstable. This instability perhaps facilitates the conformational changes in the various components that are necessary for the fusion reaction to proceed efficiently, but it greatly complicates the task of isolating the native complex in purified form. Put simply, the native complex falls apart before it can be purified, leaving only the dissociated subunits.

### Elicitation of Antibody Production by HIV-1 Env Immunogens

Because of their location on the virion surface and central role in mediating viral entry, the HIV envelope glycoproteins encoded by the viral env gene provide important targets for HIV vaccine development. However, eliciting broadly reactive and potent neutralizing antibodies (NAb) by vaccination with any form of env is problematic, because of the protective mechanisms that the native Env complex has evolved to shield its most critical sites and to limit its overall immunogenicity (reviewed by Desrosiers, 2004; Garber et al., 2004). Thus, although most HIV-infected individuals mount a robust antibody (Ab) response to the envelope glycoproteins, most anti-gp120 and anti-gp41 antibodies produced during natural infection bind weakly or not at all to virions and are thus functionally ineffective. These antibodies are probably elicited and affinity matured against “viral debris” comprising gp120 monomers or improperly processed oligomers released from virions or infected cells (Burton, 1997).

Several preventive HIV-1 subunit vaccines have been tested in Phase I and II clinical trials and a multivalent formulation has entered Phase III testing (Garber et al., 2004; Graham, 2002; McMichael and Hanke, 2003). These vaccines have contained either monomeric gp120 or unprocessed gp160 proteins, and have mostly been derived from TCLA viruses, i.e., viruses adapted to grow to high levels in immortalized T cell lines. These vaccines have consistently elicited antibodies which neutralize the homologous strain of virus and some additional TCLA viruses (Parren et al., 1999). However, the antibodies do not potently neutralize primary HIV-1 isolates (Mascola et al., 1996) and they failed to elicit any protection against HIV-1 infection when they were tested in two efficacy trials (Cohen, 2003). Compared with TCLA strains, the more clinically relevant primary isolates typically possess a different cellular tropism, show a different pattern of coreceptor usage, and have reduced sensitivity to neutralization by soluble CD4 and antibodies. These differences primarily map to the viral envelope glycoproteins (Moore et al., 1995). Hence many research groups worldwide are now trying to develop Env-based immunogens that might perform better than gp120 monomers.

There are two major obstacles to the success of a NAb-based vaccine: HIV-1 sequence variation and the defenses that the Env complex has evolved to thwart the humoral immune system. Sequence variation may turn out to be the more difficult problem to solve, but it would only become relevant if researchers first learn how to elicit antibodies capable of successfully countering even a small fraction of circulating strains (Garber et al., 2004; Burton et al., 2004). The defenses of Env against antibodies have two principal, but interlocking functions: to limit the induction of NAb in the first place, and to then reduce the probability that any elicited antibodies can bind to the functional Env complex on the virion surface, and thereby neutralize infectivity. Components of the defenses include the extensive glycosylation of both the gp120 and gp41 subunits, particularly gp120 since half its molecular weight is attributable to N-linked sugars; the structural flexibility of the CD4 and coreceptor binding sites on gp120; the limited availability of T-helper epitopes on Env; the creation of the coreceptor binding site on gp120 only after CD4 binding, combined with its inaccessibility after the Env complex has attached to cell surface CD4; and the limited exposure of conserved gp41 structural elements that are formed only during the fusion process itself (Burton et al., 2004; Grundner et al., 2004; Johnson and Desrosiers, 2002). The net effect is that only a few anti-Env antibodies can bind to pre-existing or neo-epitopes on the Env complex before or during the fusion process. The broadly reactive NAb that have been identified were isolated from HIV-1-infected humans. Although it can never be certain what forms of Env they were raised against in vivo, one possibility is that the stimulating antigen was a native structure present on virus particles or virus-infected cells (Burton et al., 2004; Parren et al., 1997b; Parren et al., 1999).

The importance of Oligomerization in Envelope Glycoprotein Structure

It has become apparent that current-generation HIV subunit vaccines do not adequately present key neutralization epitopes as they appear on virions (Parren et al., 1997). The native structure of virions is known to affect the presentation or antibody epitopes in several ways. First, much of the surface area of gp120 and gp41 is occluded by inter-subunit interactions within the trimer. Hence, several regions of gp120, especially around the N- and C-termini, that are well exposed (and highly immunogenic) on the monomeric form of the protein, are completely inaccessible on the native trimer (Moore et al., 1994a). This means that a subset of antibodies raised to gp120 monomers are irrelevant, whether they arise during natural infection (because of the shedding of gp120 monomers from virions or infected cells) or after gp120 subunit vaccination. Incidentally, this provides one of several levels of protection for the virus; the immune system is decoyed into making antibodies to shed gp120 that are poorly reactive, and hence ineffective, with virions.

A second, more subtle factor is that the structure of key gp120 epitopes can be affected by oligomerization. A classic example is provided by the epitope recognized by the broadly neutralizing, human monoclonal antibody (MAb) IgG1b12 (Burton et al., 1994). This epitope overlaps the CD4-binding site on gp120 and is present on monomeric gp120. However, IgG1b12 reacts far better with native, oligomeric gp120 than might be predicted from its monomer reactivity, which accounts for its unusually potent neutralization activity. Thus, the IgG1b12 epitope is oligomer-dependent, but not oligomer-specific.

Unfortunately, the converse situation is more common. Thus, many antibodies that are strongly reactive with CD4 binding site-related epitopes on monomeric gp120 fail to react with the native trimer and consequently do not neutralize the virus. In some undefined way, oligomerization of gp120 adversely affects the structures recognized by these MAbs (Fouts et al., 1997).

A third example of the influence of the native structure of HIV-1 envelope glycoproteins on epitope presentation is provided by the activity of gp41 MAbs. Only a single gp41 MAb (2F5) is known to have strong neutralizing activity against primary viruses (Trkola et al., 1995), and among those tested, 2F5 alone is thought to recognize an intact, gp120-gp41 complex (Sattentau et al., 1995). All other gp41 MAbs that bind to virions or virus-infected cells probably react with fusion-incompetent gp41 structures from which gp120 has dissociated. Since the most stable form of gp41 is this post-fusion configuration (Weissenhorn et al., 1997), it can be supposed that most anti-gp41 antibodies are raised (during natural infection or after gp160 vaccination) to an irrelevant gp41 structure that is not present on the pre-fusion form.

Despite the operation of these protective mechanisms, most HIV-1 isolates are potently neutralized by a limited subset of broadly reactive human MAbs, which suggests that induction of a relevant humoral immune response is not impossible. MAb IgG1b12 blocks gp120-CD4 binding; a second MAb, 2G12 (Trkola et al., 1996), acts mostly by steric hindrance of virus-cell attachment; and 2F5 acts by directly compromising the fusion reaction itself. Critical to understanding the neutralization capacity of these MAbs is the recognition that they react preferentially with the fusion-competent, oligomeric forms of the envelope glycoproteins, as found on the surfaces of virions and virus-infected cells (Parren et al., 1998). This distinguishes them from their less active peers. The limited number of MAbs that are oligomer-reactive explains why so few can neutralize primary viruses. Thus, with rare exceptions, neutralizing anti-HIV antibodies are capable of binding infectious virus while non-neutralizing antibodies are not (Fouts et al., 1998). Neutralizing antibodies also have the potential to clear infectious virus through effector functions, such as complement-mediated virolysis.

### Modifying the Antigenic Structure of the HIV Envelope Glycoproteins

HIV-1 has evolved sophisticated mechanisms to shield key neutralization sites from the humoral immune response. For example, conserved regions of gp120 involved in receptor binding are shielded by variable loops and by extensive glycosylation. The CD4 binding site (CD4BS) is recessed, and the coreceptor binding site is only formed or exposed for a short period after CD4 has already bound, thereby limiting the time and space available for antibody interference (Moore and Binley, 1997; Moore and Ho, 1995; Oloffson and Hansen, 1998; Wyatt et al., 1998).

In principle, these mechanisms can be “disabled” in a vaccine. For example, the V3 loop, which for TCLA viruses in particular is an immunodominant epitope that directs the antibody response away from more broadly conserved neutralization epitopes, can be deleted. HIV-1 is also protected from humoral immunity by the extensive glycosylation of gp120. When glycosylation sites were deleted from the V1/V2 loops of SIV gp120, not only was a neutralization-sensitive virus created, but the immunogenicity of the mutant virus was increased so that a better immune response was raised to the wild-type virus (Reitter et al., 1998). Similarly, removing the V1/V2 loops from HIV-1 gp120 renders the conserved regions underneath more vulnerable to antibodies (Cao et al., 1997), although it is not yet known whether this will translate into improved immunogenicity.

It is noteworthy that the deletion of the V1, V2 and V3 loops of the envelope glycoproteins of a TCLA virus did not improve the induction of neutralizing antibodies in the context of a DNA vaccine (Lu et al., 1998). However, the instability of the gp120-gp41 interaction, perhaps exacerbated by variable loop deletions, may have influenced the outcome of this experiment.

One approach to overcoming the so-called “neutralizing antibody problem” is to try to make, then modify, proteins that mimic the native, trimeric Env structures that exist on the virion surface (Burton et al., 2004). These trimers are, however, not easy to create in recombinant form, and several steps need to be taken to facilitate the process. The approach adopted in the present series of experiments to improving upon the immune response elicited during natural infection is to produce stabilized envelope proteins which can significantly increase the time that the native, trimeric gp120-gp41 complex is presented to the immune system.

### Native and Non-Native Oligomeric Forms of HIV Envelope Glycoproteins

Current data suggest that on the HIV virion three gp120 moieties are non-covalently associated with three, underlying gp41 components in a meta-stable configuration whose fusion potential is triggered by interaction with cell surface receptors. This pre-fusion form may optimally present neutralization epitopes. This form of the envelope glycoproteins is referred to herein as native gp120-gp41. However, other oligomeric forms are possible; these are described below and illustrated in FIG. 1.

gp160: This is a full-length gp160 precursor molecule which often aggregates when expressed as a recombinant protein. It may also be formed as a result of a mutation that prevents the processing of the gp160 precursor to gp120/gp41 (VanCott et al., 1997). The gp160 precursor does not mediate virus-cell fusion and is a poor mimic of fusion-competent gp120/gp41. When evaluated in humans, recombinant gp160 molecules offered no advantages over gp120 monomers (Gorse et al., 1998).

Uncleaved gp140 (gp140UNC): This molecule is made by eliminating the natural proteolytic site needed for cleavage of the gp160 precursor protein into gp120 and gp41 (Berman et al., 1989; Earl et al., 1990), and inserting a stop codon within the env gene to truncate the protein immediately prior to the transmembrane-spanning segment of gp41. The protein lacks the transmembrane domain and the long, intra-cytoplasmic tail of gp41, but retains the regions important for virus entry and the induction of neutralizing antibodies. The ˜140 kDa secreted protein contains full-length gp120 covalently linked through a peptide bond to the ectodomain of gp41 (gp41_{ECTO}). It forms higher molecular weight noncovalent oligomers, likely through interactions mediated by the gp41_{ECTO} moieties. Several studies have suggested that some versions of gp140UNC proteins do offer a modest but significant improvement for NAb induction, compared to monomeric gp120 proteins (VanCott et al., 1997; Yang et al., 2001). However, care must be taken when judging the magnitude and meaning of incremental improvements in Env immunogenicity, taking into account the now widespread use of new assays that offer a modest increase in sensitivity (Moore and Burton, 2004). Whether cleavage, or the lack of it, has enough influence on the conformation of gp140 trimers to significantly alter their immunogenicity has not yet been determined, but is worth evaluating.

Cleavable but uncleaved gp140 (gp140NON): During biosynthesis, gp160 is cleaved into gp120 and gp41 by a cellular endoprotease of the furin family. When over-expressed in mammalian cells, the envelope glycoproteins can saturate the endogenous furin enzymes and be secreted in precursor form. Since these molecules are potentially cleavable, they are referred to herein as gp140NON. Like gp140UNC, gp140NON migrates in SDS-PAGE with an apparent molecular mass of approximately 140 kDa under both reducing and nonreducing conditions. gp140NON appears to possess the same non-native topology as gp140UNC.

Cleaved gp140 (gp140CUT): gp140CUT refers to full-length gp120 and gp41_{ECTO} fully processed and capable of forming oligomers as found on virions. The noncovalent interactions between gp120 and gp41 are sufficiently long-lived for the virus to bind and initiate fusion with new target cells, a process which is likely completed within minutes during natural infection. The association has, however, to date proven too labile for the production of significant quantities of cleaved gp140s in near-homogenous form.

### Stabilization of HIV Envelope Glycoproteins

The metastable pre-fusion conformation of viral envelope proteins such as gp120/gp41 has evolved to be sufficiently stable so as to permit the continued spread of infection yet sufficiently labile to readily allow the conformational changes required for virus-cell fusion. For the HIV isolates examined thus far, the gp120-gp41 interaction has proven too unstable for preparative-scale production of gp140CUT as a secreted protein.

Based on the chosen strategy of attempting to make a stabilized, native Env complex that would increase the time that the trimeric gp120-gp41 complex is presented to the immune system, it has previously been reported that the association between gp120 and gp41_{ECTO} can be stabilized by the introduction of a correctly positioned intermolecular disulfide bond to make a soluble form of Env, SOS gp140 (U.S. Pat. No. 6,710,173; Binley et al., 2000; Sanders et al., 2000). In the presence of cotransfected furin, the peptide bond linking gp120 to gp41_{ECTO} is cleaved, allowing the production of properly processed gp140, designated SOS gp140. It was initially reported that oligomeric proteins were present in supernatants from 293T cells transiently expressing SOS gp140 (Binley et al., 2000). However, these oligomers were not abundant, and they did not survive purification. Thus, purified SOS gp140 is a monomeric protein (Schulke et al., 2002).

The gp41-gp41 interactions in SOS gp140 have been further stabilized by deleting the first and second variable (V1 and V2) loops (Sanders et al., 2000; PCT International Application Publication No. WO 03/022869), and by introducing amino acid substitutions into the N-terminal heptad repeat region around position 559 of gp41 (PCT International Application Publication No. WO 03/022869; Sanders et al., 2002a; see FIGS. 2-4). The SOS gp140 proteins lacking either the V1 or V2 variable loops contain a greater proportion of oligomers than the full-length protein, and the V1V2 double loop-deletant is expressed primarily as noncovalently-associated trimers. One hypothesis is that the extended and extensively glycosylated variable loops sterically impede the formation of stable gp41-gp41 interactions in the context of the full-length SOS gp140 protein.

It is also hypothesized that the amino acid substitutions around position 559 destabilize the postfusion form of the protein and thereby render the transition to this configuration less likely (see FIG. 3). One such substituted SOS gp140 protein, designated SOSIP gp140, is characterized by an I559P substitution. SOSIP gp140 is properly folded, proteolytically cleaved, substantially trimeric, and has appropriate receptor binding and antigenic properties. The SOSIP gp140 trimer can be converted to the monomeric form by heat or anionic detergents but is partially resistant to nonionic detergents.

### Particle Vaccines

Studies have revealed that converting a soluble protein into a particulate form is advantageous for vaccine preparation. Precipitated aluminum salts or “alum” remain the only adjuvant utilized in vaccines that are licensed for human use by the United States Food and Drug Administration (FDA). However, several other particulate adjuvants, including beads prepared from poly(lactic-co-glycolic acid) [PLG] (Cleland et al., 1994; Hanes et al., 1997; Powell et al., 1994), polystyrene (Kovacsovics-Bankowski and Rock, 1995; Raychaudhuri and Rock, 1998; Rock and Clark, 1996; Vidard et al., 1996), liposomes (Alving et al., 1995), calcium phosphate (He et al., 2000), and cross-linked or crystallized proteins (Langhein and Newman, 1987; St. Clair et al., 1999), have also been tested in animals.

In one series of studies, ovalbumin was linked to polystyrene beads (Vidard et al., 1996). These studies revealed that antigen-specific B cells can bind particulate antigens directly via their surface 1g receptor, enabling them to phagocytose the antigen, process it, and present the resulting peptides to T cells. The optimum size for particulate antigen presentation in this context was found to be 4 μm. Other studies with biodegradable PLG microspheres between 1 and 10 um in diameter show that these particles are capable of delivering antigens into the major histocompatibility complex (MHC) class I pathway of macrophages and dendritic cells and are able to stimulate strong cytotoxic T lymphocyte (CTL) responses in vivo (Raychaudhuri and Rock, 1998). PLG microspheres containing internalized ovalbumin and other antigens also induced humoral immune responses that were greater than those achieved with soluble antigen alone (Men et al., 1996; Partidos et al., 1996).

The potent, long-lasting immune responses induced after a single immunization with antigen-loaded or antigen-coated microspheres may result from multiple mechanisms: efficient phagocytosis of the small (<10 μm) particles, which results in their transport to lymph nodes, antigen processing and presentation to T-helper cells; the gradual release of antigens from the surface or interior of the particles, leading to the stimulation of immune-competent cells; and the sustained presentation of surface antigen (Coombes et al., 1999; Coombes et al., 1996; O'Hagan et al., 1993). Antigen-presenting cells (APCs) localize to antigen-specific B cells under these conditions, and release cytokines that increase specific antibody production and augment the expansion of these antigen specific B-cell clones. Particulate antigens are also useful for generating mucosal humoral immunity by virtue of their ability to induce secretory IgA responses after mucosal vaccination (O'Hagan et al., 1993; Vidard et al., 1996).

Overall, the use of particulate antigens allows for the simultaneous activation of both the humoral and cell-mediated arms of the immune response by encouraging the production of antigen-specific antibodies that opsonize particulate antigens and by causing the antigens to be phagocytosed and shunted into the MHC Class I antigen presentation pathway (Kovacsovics-Bankowski and Rock, 1995; Raychaudhuri and Rock, 1998; Rock and Clark, 1996; Vidard et al., 1996).

Typically, the antigens are attached to the particles by physical adsorption. Antigens have also been incorporated into particles by entrapment, as is commonly performed for PLG-based vaccines (Hanes et al., 1997). More rarely, the antigens are covalently linked to functional groups on the particles (Langhein and Newman, 1987).

This application provides assessments of the immunogenicity of cleaved, trimeric JR-FL SOSIP gp140 proteins in rabbits, using a DNA-prime, protein-boost vaccination regimen.

### SUMMARY OF THE INVENTION

The present invention provides a method for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a method for preventing a subject from becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-1.

This invention further provides a method for reducing the likelihood of a subject becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-1.

This invention still further provides a method for preventing or delaying the onset of, or slowing the rate of progression of, an HIV-1-related disease in an HIV-1-infected subject which comprises administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

In addition, the present invention provides a method for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a superparamagnetic particle and a trimeric protein complex so affixed thereto as to permit recognition of the complex by a subject's immune system, wherein in the trimeric complex each monomeric polypeptide unit comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a method for preventing a subject from becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-1.

This invention further provides a method for reducing the likelihood of a subject becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-1.

This invention still further provides a method for preventing or delaying the onset of, or slowing the rate of progression of, an HIV-1-related disease in an HIV-1-infected subject which comprises administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

This invention additionally provides a use of a nucleic acid prime and a protein boost composition for the manufacture of separate coadministerable medicaments for use in a regimen for eliciting an immune response in a subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically or therapeutically effective dose of a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a use of a nucleic acid prime and a protein boost composition for the manufacture of separate coadministerable medicaments for use in a regimen for preventing a subject from becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention further provides a use of a nucleic acid prime and a protein boost composition for the manufacture of separate coadministerable medicaments for use in a regimen for reducing the likelihood of a subject becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention still further provides a use of a nucleic acid prime and a protein boost composition for the manufacture of separate coadministerable medicaments for use in a regimen for preventing or delaying the onset of, or slowing the rate of progression of, an HIV-1-related disease in an HIV-1-infected subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a therapeutically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

In addition, this invention provides a use of a nucleic acid prime and a protein boost composition for the manufacture of separate coadministerable medicaments for use in a regimen for eliciting an immune response in a subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically or therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a use of a nucleic acid prime and a protein boost composition for the manufacture of separate coadministerable medicaments for use in a regimen for preventing a subject from becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein. each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention further provides a use of a nucleic acid prime and a protein boost composition for the manufacture of separate coadministerable medicaments for use in a regimen for reducing the likelihood of a subject becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

Finally, this invention provides a use of a nucleic acid prime and a protein boost composition for the manufacture of separate coadministerable medicaments for use in a regimen for preventing or delaying the onset of, or slowing the rate of progression of, an HIV-1-related disease in an HIV-1-infected subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

### BRIEF DESCRIPTION OF THE FIGURES

FIG. 1. Possible configurations of the HIV-1 envelope glycoproteins. i) Monomeric gp120; ii) Proteolytically uncleaved gp140 trimer with the peptide bond maintained between gp120 and gp41 (gp140UNC or gp140NON); iii) Full-length recombinant (uncleaved) gp160; iv) SOS gp140 protein, a proteolytically processed gp140 stabilized by an intermolecular disulfide bond and having a “native” configuration; and v) Native, virion-associated gp120-gp41 trimer. “Antigenicity” refers to the exposure or accessibility of important virion epitopes (i.e., epitopes accessible on the native Env complex); “coreceptor binding” refers to the ability of the Env to bind to fusion coreceptors such as the chemokine receptors CCR5 and CXCR4; “immunogenicity” refers to the ability of the envelope proteins, injected into an animal, to generate HIV neutralizing Abs; “optimization” refers to procedures that are likely to improve immunogenicity and improve production of Env proteins that mimic the native structure. Parentheses indicate predicted results.

FIG. 2. Mutations introduced into the Env Protein of HIV-1_{JR-FL} The various residues of the JR-FL gp140WT protein that have been mutated to cysteines in one or more mutants are indicated by closed arrows on the schematics of the gp120 and gp41_{ECTO} subunits. The positions of the alanine-501 and threonine-605 residues that are both mutated to cysteine in the SOS gp140 protein are indicated by the larger, closed arrows. (Note: the indicated numbering is based on the amino acid sequence of the Env in HIV-1_{HXB2}; the equivalent residues in the HIV-1_{JR-FL} Env are alanine-492 and threonine-596.) The isoleucine-559 residue mutated to proline in the SOSIP gp140 protein is labeled. The residues at the a and d positions in the N-terminal heptad repeat of gp41_{ECTO}, depicted as shaded in the secondary structure, were substituted. Major MAb epitopes are indicated (Earl et al., 1997; Parker et al., 2001; Xu et al., 1991; Zwick et al., 2001). The open boxes at the C-terminus of gp120 and the N-terminus of gp41 indicate the regions that are mutated in the gp140UNC protein to eliminate the cleavage site between gp120 and gp41. The sequence deleted in the ΔV1V2* mutant is shown in the inset.

FIG. 3. Rationale for introducing amino acid substitutions in gp41 to stabilize the trimer. (A) Model of gp41_{ECTO} and its transitions during fusion. Left panel: The hypothetical, native pre-fusion configuration of gp41 (Hunter, 1997). Middle panel: The pre-hairpin intermediate form. Right panel: The post-fusion state. In the pre-fusion configuration, the N-terminal helix is not present and the region around position 559-569 is not helical. The I559P and related substitutions are proposed to disrupt either the formation of the N-terminal helix in the pre-hairpin intermediate, or the formation of the 6-helix bundle (WO 03022869; Sanders et al., 2002a). By doing so, the modified SOS gp140 proteins are maintained in the pre-fusion configuration. The position of the T605C substitution that creates the SOS gp140 protein is also specified, as is the adjacent intermolecular disulfide bond (vertical cross-link) and the position of N-linked glycans. Only the two helices from one gp41 molecule are shown, for clarity. (B) A schematic representation of a cross-section of the 6-helix bundle, post-fusion form of gp41_{ECTO}, and a helical wheel representation of one N-terminal helix, are depicted. The residues at the a and d positions of the N-terminal heptad repeat (shaded) form the trimer interface.

FIG. 4. Stabilization of the SOS gp140 trimer by deletion of the V1 and V2 loops or mutation of Ile-559 to Pro. The oligomeric nature of the HIV-1_{JR-FL} envelope glycoprotein present in supernatants of cell cultures (A) stably transfected or (B) transiently transfected with various SOS constructs containing changes at position 559 was investigated. The unfractionated supernatants were analyzed by BN-PAGE followed by Western blotting. The gp140UNC protein was included for comparison.

FIG. 5. DNA Prime/Protein Boost Immunization regimen in rabbits. The DNA prime/protein boost study was performed using in-vivo electroporation at the facilities of Aldevron LLC (Fargo, N. Dak.). The designs of the gp120 Pilot study and Second-stage SOSIP study are shown. DNA prime: Empty Vector, plasmid PPI4 without any insert; gp140 wt, plasmid PPI4 with wild type gp140 as insert; SOSIP (=SOSIP.R6), plasmid PPI4 with cleavage-enhanced SOSIP gp140 as insert; SOS-T (=SOS.R6(T)), plasmid PPI4 with membrane-bound version of SOS gp140 as insert; gp120, purified monomeric gp120; SOSIP Trimer, purified trimeric SOSIP gp140; SOSIP Beads, trimeric SOSIP affixed to 2G12-beads; Beads only, 2G12-beads without SOSIP.

FIG. 6. Schematic representation of a method for immunopurification of gp120 or SOS trimers with magnetic microbeads. Abs that bind to gp120 or SOSgp140 trimers are captured onto magnetic Protein A or Protein G microbeads, followed by the binding of gp120 or SOSgp140 trimers to the affixed Abs. The magnetically labeled gp120 or SOSgp140 trimers is then isolated using a magnet.

FIG. 7. Timelines of the Pilot study (A) and the Second-stage SOSIP study (B). DNA immunizations are indicated by open arrows, protein immunizations by solid arrows, and bleeds by shaded arrows below the horizontal line.

FIG. 8. Design of a trimer-binding ELISA for evaluating neutralizing activity of antibodies. In this ELISA format, the mutant HIV Env is biospecifically captured onto the wells of a microtiter plate. The test antibodies, elicited in a subject by administration of an Env immunogen, are added to the plate in a range of concentrations. Specifically bound antibodies are detected using an enzyme-linked reporter antibody (e.g., an alkaline phosphatase-labeled anti-IgG Ab that is specific for the subject's antibodies) and a colorimetric substrate.

FIG. 9. Identification of oligomeric HIV envelope by sCD4 capture ELISA. Envelope proteins (30 ng/well) were captured to a microtiter plate with sCD4 (5 ng/ml) and detected with saturating amounts (20 ng/ml) of the MAbs 2G12, CD4-IgG2 and IgG1b12.

FIG. 10. Analysis of Env antigenicity by lectin-capture ELISA. Envelope proteins (30 ng/well) were captured to a microtiter plate with lentil lectin (10 ng/ml) and detected with increasing amounts of the MAbs 2G12, 2F5 and IgG1b12.

FIG. 11. SOSIP-immunized rabbits generate antibodies that preferentially react with trimeric Env. Midpoint titer ratios were calculated for sera obtained at weeks 20, 30, 41 and 54 from rabbits immunized with gp120 or SOSIP. The individual sera were analyzed for reactivity with gp120 monomer or SOSIP trimer in a lentil-lectin-capture ELISA. Data are represented as the ratio of midpoint titers observed against SOSIP gp140 and gp120. The antigens used for immunization are indicated below the figure. SOS-T=membrane-bound SOS gp140; gp140=wild-type gp140; t-SOSIP=purified SOSIP trimers; T-SOSIP beads=purified SOSIP trimers captured onto 2G12-coated paramagnetic beads. SOSIP/gp120 ratios of >2 indicate enhanced binding to the trimeric envelope.

FIG. 12. Analysis of Env antigenicity by immunoprecipitation. (A) The antigenic structure of the monomeric gp120, monomeric SOSIP gp140, trimeric uncleaved (REKR->IEGR) gp140 and trimeric SOSIP gp140 proteins was analyzed in immunoprecipitation assays using the neutralizing and non-neutralizing anti-gp120 mAbs indicated. (B) The antigenic structure of monomeric gp120, monomeric SOSIP gp140, trimeric uncleaved (REKR->IEGR) gp140 and trimeric SOSIP gp140 proteins was analyzed in immunoprecipitation assays using the neutralizing (2F5 and 4E10) and non-neutralizing (D50, D49 and T4) anti-gp41 mAbs indicated.

FIG. 13. Env constructs and SOSIP.R6 trimer purification. (A) Schematic of the env DNA constructs used for the priming immunization. Highlighted are the approximate size of the Env protein construct relative to full length Wt gp160, the inclusion of a Wt (REKR) or enhanced (RRRRRR) cleavage site motif, the approximate position of the SOS cysteine bond (C═C), and the approximate position of the gp41-gp41 trimer stabilizing mutation (I559P). The C1-C5 domains of gp120 are also identified. The Ecto, TM and Cyt labels indicate the positions of the gp41 ectodomain, the membrane-spanning domain and the cytoplasmic tail, respectively. (B) SDS-PAGE analysis of the purified HIV-1 gp120 and SOSIP.R6 gp140 proteins. The purified proteins, solubilized in Laemmli sample buffer with (reduced) or without (non-reduced) 20 mM dithiothreitol (DTT), were resolved on a 4-12% Bis-Tris polyacrylamide gradient gel and stained using Coomassie Blue. (C) Blue Native PAGE analysis of the purified HIV-1 gp120 and SOSIP.R6 gp140 proteins. The proteins were resolved on a 4-12% Bis-Tris polyacrylamide gradient gel and visualized using Coomassie Blue.

FIG. 14. Anti-gp120 binding antibodies elicited by immunization in the Pilot study. (A) The schematic for the Pilot study highlights the DNA construct (gp140 Wt, or “empty vector”) used for priming (open arrows), the dose of gp120 protein (10, 30 or 100 μg) used for each of the protein boosts (filled arrows), and the serum collection times (grey arrows). (B) Generation of anti-gp120 antibodies in the Pilot study. Rabbits were primed with 1 mg of pPPI4 expressing either codon-optimized soluble gp140 Wt Env (filled symbols) or the “empty vector” control (open squares), then boosted with 10 μg (filled triangles), 30 μg (filled squares) or 100 μg (filled circles) of soluble gp120, at the times indicated. The anti-gp120 antibody responses were measured by ELISA. Each datum point represents the mean (n=2 animals) midpoint anti-gp120 binding titer for each Arm.

FIG. 15. Anti-gp120 binding antibodies elicited by SOSIP.R6 immunization. (A) The schematic for the second-stage study highlights the DNA construct (“empty vector”, SOSIP.R6 or SOS.R6(T)) used for priming (open arrows) the protein construct (SOSIP.R6 Trimer, SOSIP.R6 Trimer coupled to paramagnetic beads, or “empty” beads) used for each of the protein boosts (filled arrows), and the serum collection times (grey arrows). The designation numbers for the individual animals in each arm (A-E) are also listed. (B) Rabbits were primed with 1 mg pPPI4 expressing soluble gp140 SOSIP.R6Env (▴,□,Δ; arms B, C, D) or membrane-bound SOS.R6 Env (; arm E), or primed with the “empty vector” control (▪; arm A). The animals were then boosted with 30 μg of SOSIP.R6 Trimer (▪,▴,; arms A, B, E), 30 pg of SOSIP.R6 Trimer coupled to beads (A; arm D), or with empty beads as a control (□; arm C), at the times indicated. The anti-gp120 antibody responses were measured by ELISA. Each datum point represents the mean (n=4 animals) midpoint anti-gp120 binding titer for each Arm. The mean (±SD) anti-gp120 titer in the gp140 Wt DNA-primed and gp120-boosted animals (5695-1 to 5695-6) at week 20 of the Pilot study is indicated by the dotted lines, for comparison purposes.

FIG. 16. Neutralization of HIV-1_{JR-FL} and HIV-1_{MN} Env-pseudotype viruses by selected sera. A panel of antisera taken from 10 rabbits immunized with trimeric SOSIP.R6 Env (Second-stage study) was assembled, to assess the generation of neutralizing antibodies over time. Neutralization titers against (A, B) Env-pseudotyped HIV-1_{MN} and (C) Env-pseudotyped HIV-1JR-FL were determined at 12 time points over the 54 week time course, using (A, C) 50% or (B) 90% endpoints. Symbols are used to represent the following groups: “empty vector” primed/SOSIP.R6 Trimer boost (squares), SOSIP.R6 prime/SOSIP.R6 Trimer boost (triangles), SOSIP.R6 prime/bead-SOSIP.R6 Trimer boost (circles) and membrane-bound SOS.R6(T) prime/SOSIP.R6 Trimer boost (diamonds). The timings of DNA prime (open arrows) and protein boost (filled arrows) events are indicated. Note that the rabbit sera were not titrated beyond a dilution of 1:160; hence differences between animals, or the height of the peaks and troughs, cannot be determined for values beyond this level.

FIG. 17. 50% neutralization titers against envelope-pseudotyped viral particles. Serial dilutions of rabbit sera were incubated with HIV-1 Env-Luc-pseudotyped viruses and then combined with U87-CD4-coreceptor reporter cells. Relative infectivity was detected by luciferase expression and compared to infectivity in the presence of pre-immune serum. The best 10 animal sera are shown. ADA and YU2 pseudoviruses were neutralized <50% at the lowest dilution of sera (1:10). Infectivity of MLV control pseudoviruses was not affected by the sera.

FIG. 18. 70% neutralization titers against envelope-pseudotyped viral particles. Serial dilutions of rabbit sera were incubated with HIV-1 Env-Luc-pseudotyped viruses and then combined with U87-CD4-coreceptor reporter cells. Relative infectivity was detected by luciferase expression and compared to infectivity in the presence of pre-immune serum. The best 10 animal sera are shown. ADA and YU2 pseudoviruses were neutralized by <70% at the lowest dilution of sera (1:10). Infectivity of MLV pseudoviruses was not affected by the sera.

FIG. 19. Timecourse of neutralization titers against HIV-1_{JR-FL} pseudoviruses. Rabbit sera were collected at the indicated times, serially diluted, and then incubated with JR-FL-pseudotyped viruses and U87-CD4-coreceptor cells as described in the Methods. Relative infectivity was assessed by luciferase expression. Percent neutralization was calculated on a per-dilution basis as [1−(sample RLU/pre-immune RLU)]×100.50% neutralization titers were then calculated by linear regression. The midpoint titers (50% neutralization) are presented for each bleed.

FIG. 20. Comparative neutralization of Env-pseudotyped and PBMC-grown HIV-1 (week 54). Neutralization of Env-pseudotyped or PBMC-grown HIV-1 by sera from SOSIP gp140-immunized rabbits was measured. For the PBMC-based assay, replication-competent viruses (100 TCID₅₀/well) were incubated 1 h at 37° C. with serial dilutions of the sera. Then, the virus-sera mixture was added to freshly activated PBMC (3×10⁴/well) in 96-well microtiter plates. Twenty-four hours later, cells were washed with PBS to remove the residual p24. Virus replication was measured by detection of HIV-1 p24 antigen in culture supernatants 7 days post-infection. Neutralization of Env-pseudotyped viruses was performed as described above. The reciprocal neutralization titer is the average of 3 independent assays.

FIG. 21. Neutralizing activity in the Phenosense™ HIV entry assay. Env-complemented reporter viruses were prepared using pHIVenv and a second plasmid, pHIVlucΔU3. The latter vector is a replication-defective clone of HIV-1NL4˜3 wherein env has been replaced with a luciferase expression cassette. This vector also contains a self-inactivating deletion in the U3 region of the 3′ LTR that substantially reduces viral gene transcription from the 5′ LTR in infected cells. Reporter viruses are produced by co-transfecting human embryonic kidney 293 cells with pHIVenv and pHIVlucΔU3. Viral particles are collected 48 h post-transfection and used to infect U87-CD4-CCR5 or U87-CD4-CXCR4 target cells in the presence or absence of sera. Replication is monitored by measuring luciferase activity in infected target cells approximately 72 h post-infection.

FIG. 22. Neutralization of HIV-1JR-FL pseudovirus by rabbit antisera is predominantly mediated by non-V3, gp120-directed antibodies. (A) BSA-(filled triangles), V3 peptide-(open circles) or gp120-(open diamonds) coupled CNBr-Sepharose beads were used to deplete a rabbit pre-immune serum pool spiked with (i, ii) the V3-specific MAb, PA1, or (iii) the CD4-IgG2 molecule, before assay by (i, iii) gp120 binding ELISA or (ii) V3 peptide ELISA. The non-depleted serum was also assayed (filled squares). (iv) Rabbit final bleed sera from the Pilot study, 5695-3 (filled bars), and follow-up study, 228 (open bars), 236 (half-tone bars) and 241 (oblique lined bars) were left untreated (‘Pre’) or depleted using BSA-, V3- or gp120-coupled beads before determination of midpoint binding titers against gp120 or V3 peptide by ELISA, as indicated. (B) Untreated and bead-treated sera 5695-3 and 241 were also used in pseudovirus neutralization assays against HIV-1JR-FL (upper panels) or HIV-1MN (lower panels). Bars represent serum dilutions of 1:10 (filled bars), 1:40 (grey bars) and 1:160 (open bars). Representative data were derived from 2-3 experiments.

FIG. 23. Stability of SOSIP gp140 in rabbit serum. The antigenic structures of monomeric gp120 and trimeric SOSIP gp140 were analyzed in immunoprecipitation assays after incubation of the purified proteins in 100% rabbit serum overnight at 37° C. in the presence of adjuvant. Percent reactivity reflects the ratio of the amounts of envelope protein immunoprecipitated from treated and untreated samples. *: Treatment had no measurable effect on the amount of protein immunoprecipitated by the indicated antibody.

FIG. 24. HIV-1_{JR-FL} neutralization is not mediated by antibodies to the V3-loop. Selected rabbit sera from the terminal bleed were depleted of antibodies against the JR-FL V3-loop (with peptide-beads) or gp120 (with recombinant protein-beads). Bound antibodies were eluted with glycine buffer (pH 2.5-3.0), neutralized with Tris buffer, diafiltered into phosphate-buffered saline (PBS), and adjusted to the original volume of the sera. The depleted sera or the antibody fractions were assayed for neutralization in pseudovirus assays as described in the Methods. The 50% neutralization titers are presented for each sample.

FIG. 25. Neutralizing activity of purified IgG from rabbit sera (Week 54). Total Ig was purified from final bleed rabbit sera using the T-Gel Purification kit (Pierce, Inc.) according to the manufacturer's instructions, except that an azide-free buffer (50 nM NaH₂PO4, pH 8) was used for Ab elution. The amount of recovered rabbit IgG was quantified using the Easy-Titer Rabbit IgG Assay Kit (Pierce, Inc.), the average recovery being 70% (n=10). The extent of IgG recovery for each individual serum sample was always taken account when determining neutralization or ELISA titers, to allow comparisons between purified IgG and the corresponding unfractionated serum.

FIG. 26. Immunopurification of SOSIP gp140 using magnetic beads. Purified monomeric gp120 (A) or gel filtration-purified trimeric SOSIP gp140 (B) were captured to magnetic beads with 2G12 and analyzed by SDS-PAGE and Coomassie blue staining. Known concentrations of 2G12 and gp120 were used to estimate the amount of SOSIP captured to the beads. (C) Beads-capture efficiency for SOSIP gp140 using MAbs or CD4-IgG2.

FIG. 27. Purification of trimeric SOSIP gp140 to homogeneity. SOSIP gp140 was transiently expressed from 293T cells and the trimeric species was purified to homogeneity. The purified protein was analyzed by (A) reducing and non-reducing SDS-PAGE, and (B) BN-PAGE. The proteins were visualized by Coomassie blue staining.

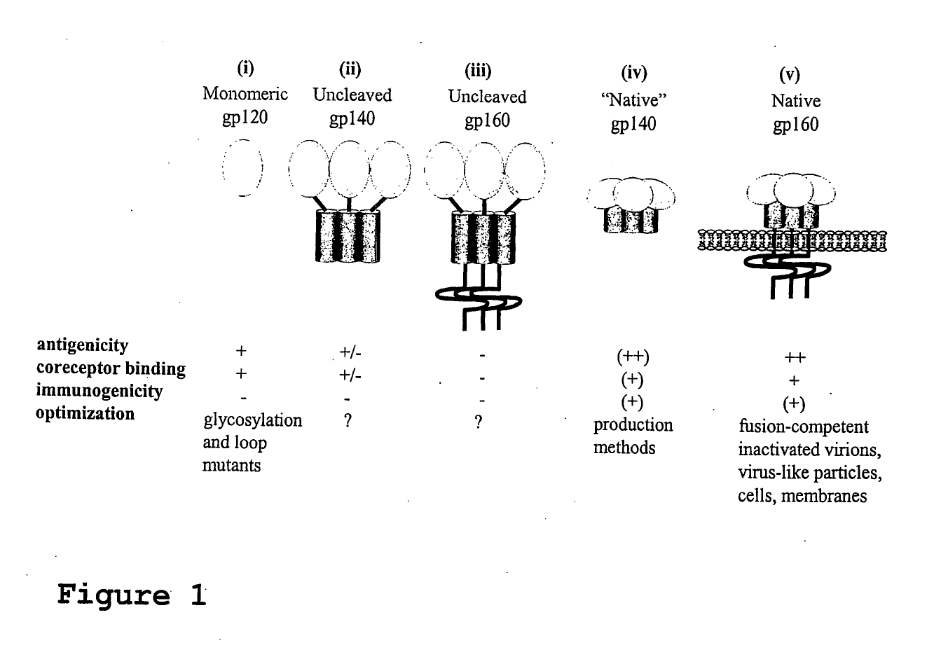

### DETAILED DESCRIPTION OF THE INVENTION

### Definitions

As used in this application, except as otherwise expressly provided herein, each of the following terms shall have the meaning set forth below.

The following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine; B=asx=asparagine or aspartic acid; Z=glx=glutamine or glutamic acid.

An “adjuvant” shall mean any agent suitable for enhancing the immunogenicity of an antigen. Numerous adjuvants, including particulate adjuvants, suitable for use with both protein- and nucleic acid-based vaccines, and methods of combining adjuvants with antigens, are well known to those skilled in the art.

“Administering” shall mean delivering in a manner which is effected or performed using any of the various methods and delivery systems known to those skilled in the art. Administering can be performed, for example, topically, intravenously, pericardially, orally, parenterally, via implant, transmucosally, transdermally, intradermally, intramuscularly, subcutaneously, intraperitoneally, intrathecally, intralymphatically, intralesionally, epidurally, or by in vivo electroporation. An agent or composition may also be administered in an aerosol, such as for pulmonary and/or intranasal delivery. Administering can also be performed, for example, once, a plurality of times, and/or over one or more extended periods.

An “A492C mutation” refers to a point mutation of amino acid 492 in HIV-1_{JR-FL} gp120 from alanine to cysteine. Because of the sequence variability of HIV, this amino acid will not be at position 492 in all other HIV isolates. For example, in HIV-1_{HXB2} the corresponding amino acid is A501 (Genbank Accession No. AAB50262) and in HIV-1_{NL4-3} it is A499 (Genbank Accession No. AAA44992). It may also be a homologous amino acid other than alanine or cysteine. This invention encompasses cysteine mutations in such amino acids, which can be readily identified in other HIV isolates by those skilled in the art.

A “boosting vaccine” comprises a composition containing the same antigen as in the priming vaccine or precursor thereof, but in a different form, wherein the boosting vaccine induces an immune response in the host. In one embodiment, the boosting vaccine comprises a recombinant soluble protein affixed to a particle.

A “canonical glycosylation site” includes but is not limited to an Asn-X-Ser or Asn-X-Thr sequence of amino acids that defines a site for N-linkage of a carbohydrate. In addition, Ser or Thr residues not present in such sequences to which a carbohydrate can be linked through an O-linkage are canonical glycosylation sites. In the latter case of a canonical glycosylation site, a mutation of the Ser and Thr residue to an amino acid other than a serine or threonine will remove the site of O-linked glycosylation.

“CCR5” is a chemokine receptor which binds members of the C—C group of chemokines and whose amino acid sequence comprises that provided in Genbank Accession Number 1705896 and related polymorphic variants. As used herein, CCR5 includes extracellular portions of CCR5 capable of binding the HIV-1 envelope protein.

“CDR” or complementarity determining region means a highly variable sequence of amino acids in the variable domain of an antibody.

A “cell” includes a biological cell, e.g., a HeLa cell, and a non-biological cell, e.g., a phospholipid vesicle or virion.

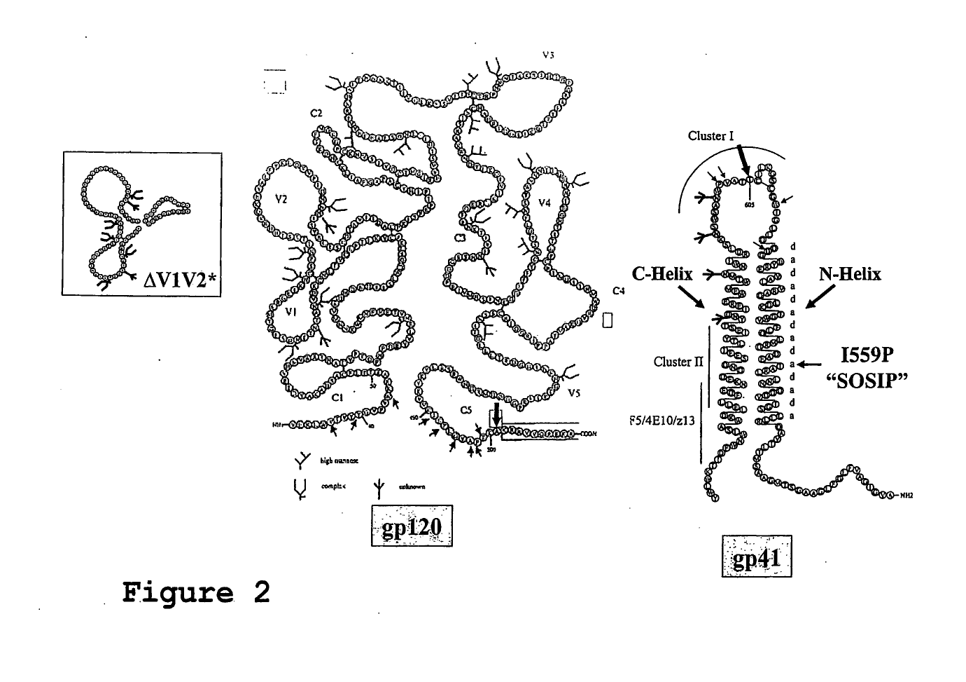

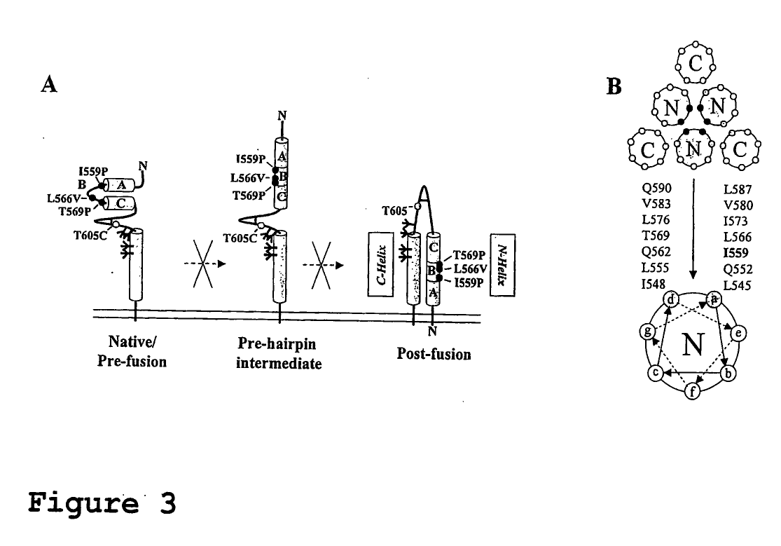

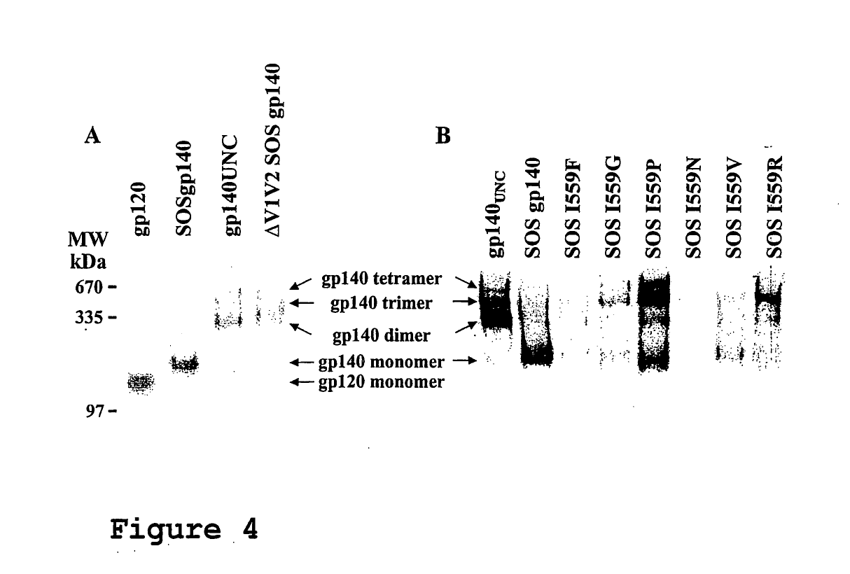

“C1 region” means the first conserved sequence of amino acids in the mature gp120 glycoprotein. The C1 region includes the amino-terminal amino acids. In HIV-1_{JR-FL}, the C1 region consists of the amino acids VEKLWVTVYYGVPVWKEATTTLFCASDAKAYDTEVHNVWATHACVPTDPNPQEVVLENVT EHFNMWKNNMVEQMQEDIISLWDQSLKPCVKLTPLCVTLN (SEQ ID NO: 1). Amino acid residues 30-130 of the sequence set forth in FIG. 2 (gp120) have this sequence. In other HIV isolates, the C1 region will comprise a homologous amino-terminal sequence of amino acids of similar length. W44C and P600C mutations are as defined above for A492 and T596 mutations. Because of the sequence variability of HIV, W44 and P600 will not be at positions 44 and 600 in all HIV isolates. In other HIV isolates, homologous, non-cysteine amino acids may also be present in the place of the tryptophan and proline. This invention encompasses cysteine mutations in such amino acids, which can be readily identified in other HIV isolates by those skilled in the art.

“C5 region” means the fifth conserved sequence of amino acids in the gp120 glycoprotein. The C5 region includes the carboxy-terminal amino acids. In HIV-1_{JR-FL} gp120, the unmodified C5 region consists of the amino acids GGGDMRDNWRSELYKYKVVKIEPLGVAPTKAKRRVVQRE (SEQ ID NO: 2). Amino acid residues 462-500 of the sequence set forth in FIG. 2 (gp120) have this sequence. In other HIV isolates, the C5 region will comprise a homologous carboxy-terminal sequence of amino acids of similar length.

“CXCR4” is a chemokine receptor which binds members of the C—X—C group of chemokines and whose amino acid sequence comprises that provided in Genbank Accession No 400654 and related polymorphic variants. As used herein, CXCR4 includes extracellular portions of CXCR4 capable of binding the HIV-1 envelope protein.

A “derivatized” antibody is one that has been modified. Methods of derivatization include, but are not limited to, the addition of a fluorescent moiety, a radionuclide, a toxin, an enzyme or an affinity ligand such as biotin.

To “enhance the stability” of an entity, such as a protein, means to make the entity more long-lived or resistant to dissociation. Enhancing stability can be achieved, for example, by the introduction of disulfide bonds, salt bridges, hydrogen bonds, hydrophobic interactions, favorable van der Waals contacts, a linker peptide or a combination thereof. Stability-enhancing changes can be introduced by recombinant methods. As used herein, “mutant” means that which is not wild-type.

“Exposed” to HIV-1 means contact with HIV-1 such that infection could result.

“gp41” shall include, without limitation, (a) whole gp41 including the transmembrane and cytoplasmic domains; (b) gp41 ectodomain (gp41_{ECTO}); (c) gp41 modified by deletion or insertion of one or more glycosylation sites; (d) gp41 modified so as to eliminate or mask the well-known immunodominant epitope; (e) a gp41 fusion protein; and (f) gp41 labeled with an affinity ligand or other detectable marker. As used herein, “ectodomain” means the extracellular region of a transmembrane protein exclusive of the transmembrane spanning and cytoplasmic regions.

“HIV” shall mean the human immunodeficiency virus. HIV shall include, without limitation, HIV-1. The human immunodeficiency virus (HIV) may be either of the two known types of HIV (HIV-1 or HIV-2). The HIV-1 virus may represent any of the known major subtypes (Classes A, B, C, D E, F, G and H) or outlying subtype (Group O).

The human immunodeficiency virus includes but is not limited to the JR-FL strain. Surface proteins include but are not limited to gp120. An amino acid residue of the C1 or C5 region of gp120 may be mutated. The HIV-1_{JR-FL} gp120 amino acid residues which may be mutated include but are not limited to the following amino acid residues: V35; Y39, W44; G462; 1482; P484; G486; A488; P489; A492; and E500. The gp120 amino acid residues are also set forth in FIG. 2. The gp41 amino acid residues which may be mutated include but are not limited to the following: D580; W587; T596; V599; and P600. The gp41 amino acid residues are also set forth in FIG. 2 (note that the numbering of amino acid residues in FIG. 2 is referenced on the Env of HIV-1_{HXB2}).

HIV-1_{DH123} is a clone of a virus originally isolated from the peripheral mononuclear cells (PBMCs) of a patient with AIDS (Shibata, 1995). HIV-1_{DH123} is known to utilize both CCR5 and CXCR4 as fusion coreceptors and has the ability to replicate in PHA-stimulated PBMCs, blood-derived macrophages and immortalized T cell lines.

HIV-1_{Gun-1} is a cloned virus originally isolated from the peripheral blood mononuclear cells of a hemophilia B patient with AIDS (Takeuchi, 1987). HIV-1_{Gun-1} is known to utilize both CCR5 and CXCR4 as fusion coreceptors and has the ability to replicate in PHA-stimulated PBMCs, blood-derived macrophages and immortalized T cell lines.

HIV-1_{HXB2} (HIV-1_{HXBc2}) is a TCLA virus that is known to utilize CXCR4 as a fusion coreceptor and has the ability to replicate in PHA-stimulated PBMCs and immortalized T cell lines but not blood derived macrophages.

HIV-1_{JR-FL} is a strain that was originally isolated from the brain tissue of an AIDS patient taken at autopsy and co-cultured with lectin-activated normal human PBMCs (O'Brien et al., 1990). HIV-1_{JR-FL} is known to utilize CCR5 as a fusion coreceptor and has the ability to replicate in phytohemagglutinin (PHA)-stimulated PBMCs and blood-derived macrophages but does not replicate efficiently in most immortalized T cell lines.

HIV-1_{89.6} is a cloned virus originally isolated from a patient with AIDS (Collman, 1992) HIV-1_{89.6} is known to utilize both CCR5 and CXCR4 as fusion coreceptors and has the ability to replicate in PHA-stimulated PBMCS, blood-derived macrophages and immortalized T cell lines.

Although certain of the above strains are used herein to test the neutralization efficacy of antibodies raised by different immunogens, other HIV-1 strains could be substituted in their place as is well known to those skilled in the art.

“HIV gp140 protein” shall mean a protein having two disulfide-linked polypeptide chains, the first chain comprising the amino acid sequence of the HIV gp120 glycoprotein and the second chain comprising the amino acid sequence of the water-soluble portion of HIV gp41 glycoprotein (“gp41 portion”). HIV gp140 protein includes, without limitation, proteins wherein the gp41 portion comprises a point mutation such as I559G, L566V, T569P and I559P. HIV gp140 protein comprising such mutations is also referred to as “HIV SOS gp140”, as well as “HIV gp140 monomer.”

“Host cells” shall include, but are not limited to, bacterial cells (including gram-positive cells), yeast cells, fungal cells, insect cells and animal cells. Suitable animal cells include, but are not limited to HeLa cells, COS cells, CV1 cells and various primary mammalian cells. Numerous mammalian cells can be used as hosts, including, but not limited to, mouse embryonic fibroblast NIH-3T3 cells, CHO cells, HeLa cells, L(tk−) cells and COS cells. Mammalian cells can be transfected by methods well known in the art, such as calcium phosphate precipitation, electroporation and microinjection. Electroporation can also be performed in vivo as described previously (see U.S. Pat. Nos. 6,110,161; 6,262,281; and 6,610,044).

“Humanized” describes antibodies wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they do not abrogate the ability of the antibody to bind a given antigen. Suitable human immunoglobulin molecules include IgG1, IgG2, IgG3, IgG4, IgA, IgE and IgM molecules. A “humanized” antibody would retain an antigenic specificity similar to that of the original antibody.

“Immunizing” means generating an immune response to an antigen in a subject. This can be accomplished, for example, by administering a primary dose of a vaccine to a subject, followed after a suitable period of time by one or more subsequent administrations of the vaccine, so as to generate in the subject an immune response against the vaccine. A suitable period of time between administrations of the vaccine may readily be determined by one skilled in the art, and is usually on the order of several weeks to months.

“I559G” shall mean a point mutation wherein the isoleucine residue at position 559 of a polypeptide chain is replaced by a glycine residue.

“I559P” shall mean a point mutation wherein the isoleucine residue at position 559 of a polypeptide chain is replaced by a proline residue.

“L566V” shall mean a point mutation wherein the leucine residue at position 566 of a polypeptide chain is replaced by a valine residue.

“Nucleic acid” shall mean any nucleic acid including, without limitation, DNA, RNA and hybrids thereof. The nucleic acid bases that form nucleic acid molecules can be the bases A, C, T, G and U, as well as derivatives thereof. Derivatives of these bases are well known in the art and are exemplified in PCR Systems, Reagents and Consumables (Perkin-Elmer Catalogue 1996-1997, Roche Molecular Systems, Inc., Branchburg, N.J., USA).

“Operably affixed”, when in reference to a trimeric complex or other antigen on a particle, means affixed so as to permit recognition of the complex or other antigen by an immune system.

“Pharmaceutically acceptable carriers” are well known to those skilled in the art and include, but are not limited to, 0.01-0.1M and preferably 0.05M phosphate buffer, phosphate-buffered saline (PBS), or 0.9% saline. Additionally, such pharmaceutically acceptable carriers may include, but are not limited to, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives; sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Preservatives and other additives, such as, for example, antimicrobials, antioxidants, chelating agents, inert gases, and the like may also be included with all the above carriers.

A “pharmaceutically acceptable particle” means any particle made of a material suitable for introduction into a subject.

“Priming” means any method whereby a first immunization using an antigen permits the generation of an immune response to the antigen upon a second immunization with the same antigen, wherein the second immune response is greater than that achieved where the first immunization is not provided. “Priming amount” means the amount of priming vaccine used.

A “prophylactically effective amount” is any amount of an agent which, when administered to a subject prone to suffer from a disorder, inhibits the onset of the disorder.

“Inhibiting” the onset of a disorder means either lessening the likelihood of the disorder's onset, or preventing the onset of the disorder entirely. In the preferred embodiment, inhibiting the onset of a disorder means preventing its onset entirely.

“Protein G microbeads” and “Protein A microbeads” described herein are small (mean diameter of 50 nm) superparamagnetic particles coated with covalently attached Protein G and Protein A, respectively. Protein G and Protein A are cell wall components of Group G Streptococci and Staphylococcus aureus, respectively. These two proteins have the ability to bind to the Fc region of most mammalian immunoglobulins but have different affinities for different subclasses of immunoglobulins in different species.

“Reducing the likelihood of a subject's becoming infected with a virus” means reducing the likelihood of the subject's becoming infected with the virus by at least two-fold. For example, if a subject has a 1% chance of becoming infected with the virus, a two-fold reduction in the likelihood of the subject becoming infected with the virus would result in the subject having a 0.5% chance of becoming infected with the virus. In the preferred embodiment of this invention, reducing the likelihood of the subject's becoming infected with the virus means reducing the likelihood of the subject's becoming infected with the virus by at least ten-fold.

“SOSIP gp140” means the SOS gp140 protein containing an I559P substitution. SOSIP gp140 is proteolytically cleaved and substantially trimeric, but can be converted to the monomeric form by heat or anionic detergents.

“Subject” means any animal or artificially modified animal. Animals include, but are not limited to, humans, non-human primates, cows, horses, sheep, pigs, dogs, cats, rabbits, ferrets, rodents such as mice, rats and guinea pigs, and birds such as chickens and turkeys. Artificially modified animals include, but are not limited to, SCID mice with human immune systems. In the preferred embodiment, the subject is a human.

“Superparamagnetic particles” are particles that exhibit magnetic properties only when placed within a magnetic field and show no residual magnetism when removed from this field.

A “therapeutically effective amount” is any amount of an agent which, when administered to a subject afflicted with a disorder against which the agent is effective, causes the subject to be treated. “Treating” a subject afflicted with a disorder shall mean causing the subject to experience a reduction, remission or regression of the disorder and/or its symptoms. In one embodiment, recurrence of the disorder and/or its symptoms is prevented. In the preferred embodiment, the subject is cured of the disorder and/or its symptoms.

“T569P” shall mean a point mutation wherein the threonine residue at position 569 of a polypeptide chain is replaced by a proline residue.

A “T596C mutation” refers to a point mutation of amino acid 596 in HIV-1_{JR-FL} gp41 from threonine to cysteine. Because of the sequence variability of HIV, this amino acid will not be at position 596 in all other HIV isolates. For example, in HIV-1_{HXB2} the corresponding amino acid is T605 (Genbank Accession No. AAB50262) and in HIV-1_{NL4-3} the corresponding amino acid is T603 (Genbank Accession No. AAA44992). It may also be a homologous amino acid other than threonine or cysteine. This invention encompasses cysteine mutations in such amino acids, which can be readily identified in other HIV isolates by those skilled in the art.

A “vector” shall mean any nucleic acid vector known in the art. Such vectors include, but are not limited to, plasmid vectors, cosmid vectors and bacteriophage vectors. For example, one class of vectors utilizes DNA elements which are derived from animal viruses such as animal papilloma virus, polyoma virus, adenovirus, vaccinia virus, baculovirus, retroviruses (RSV, MMTC or MoMLV), Semliki Forest virus or SV40 virus. The eukaryotic expression plasmid PPI4 and its derivatives are widely used in constructs described herein. However, the invention is not limited to derivatives of the PPI4 plasmid and may include other plasmids known to those skilled in the art.

In accordance with the invention, numerous vector systems for expression of recombinant proteins may be employed. For example, one class of vectors utilizes DNA elements which are derived from animal viruses such as bovine papilloma virus, polyoma virus, adenovirus, vaccinia virus, baculovirus, retroviruses (RSV, MMTV or MOMLV), Semliki Forest virus or SV40 virus. Additionally, cells which have stably integrated the DNA into their chromosomes may be selected by introducing one or more markers which allow for the selection of transfected host cells. The marker may provide, for example, prototropy to an auxotrophic host, biocide (e.g., antibiotic) resistance, or resistance to heavy metals such as copper or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals. The cDNA expression vectors incorporating such elements include those described by (Okayama and Berg, 1983).

“Virally infected” means the introduction of viral genetic information into a target cell, such as by fusion of the target cell membrane with the virus or infected cell. The target may be a cell of a subject. In the preferred embodiment, the target cell is a cell in a human subject.

“Virus-like particles” or VLPs are particles which are non-infectious in any host, nonreplicating in any host, which do not contain all of the protein components of live virus particles.

### EMBODIMENTS OF THE INVENTION

The production of stabilized, trimeric HIV-1 gp140 proteins which mimic the trimeric conformation of the native HIV-1 envelope glycoprotein complex has previously been described. The present invention relates to the use of stabilized, trimeric HIV-1 gp140 protein as an immunogen to generate neutralizing antibodies against HIV-1.

Specifically, this invention provides a first method for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

In one embodiment of the subject method, the modified gp41 comprises an I559P substitution. In another embodiment, the modified gp120 comprises an A501C mutation. In a further embodiment, the modified gp41 comprises a T605C mutation. In a still further embodiment, the modified gp120 comprises an A501C mutation and the modified gp120 is bound to the modified gp41 by a disulfide bond between the cysteine residue at position 501 of the modified gp120 and the cysteine residue at position 605 of the modified gp41.

In yet another embodiment, the protein boost further comprises a pharmaceutically acceptable carrier.

In one embodiment of the instant method, the first predefined interval is at least one week. In another embodiment, the first predefined interval is at least four weeks. In a further embodiment, the second predefined interval is the same as the first predefined interval. In a still further embodiment, the second predefined interval is at least one week. In yet another embodiment, the second predefined interval is at least four weeks. In a preferred embodiment, two doses of the nucleic acid prime are administered. In a further embodiment, the two doses of the nucleic acid prime are administered at week 0 and week 4. In a still further embodiment, six doses of the protein boost composition are administered at weeks 12, 16, 20, 28, 37 and 52.

Additionally, in one embodiment each dose of nucleic acid prime comprises 4 μg to 10 mg per kg body weight of the subject. In another embodiment, each dose of nucleic acid prime comprises 40 μg to 2.5 mg per kg body weight of the subject. In a further embodiment, each dose of nucleic acid prime comprises 150 μg to 1 mg per kg body weight of the subject. In a preferred embodiment, each dose of nucleic acid prime comprises about 400 μg per kg body weight of the subject.

In yet another embodiment, each dose of the protein boost composition comprises 0.1 μg to 1 mg per kg body weight of the subject. In a further embodiment, each dose of the protein boost composition comprises 1 to 250 μg per kg body weight of the subject. In a still further embodiment, each dose of the protein boost composition comprises 5 to 50 pg per kg body weight of the subject. In a preferred embodiment, each dose of the protein boost composition comprises about 12 pg per kg body weight of the subject.

In an additional embodiment of the first method, the nucleic acid prime is a DNA prime. In another embodiment, the DNA prime encodes wild-type gp140. In a further embodiment, the DNA prime encodes cleaved HIV-1 gp140. In a still further embodiment, the DNA prime encodes a cleavage-enhanced HIV-1 gp140. In yet another embodiment, the DNA prime encodes uncleaved HIV-1 gp140. In another embodiment, the DNA prime encodes a variant of HIV-1 gp140 comprising a gp41 component which comprises a transmembrane domain that is capable of binding to a biological membrane. In a further embodiment, the gp41 component contains three additional amino acids compared to unmodified gp41 and ends in the sequence V₆₆₆NRV₆₆₉. In an additional embodiment, the codons in the DNA prime have been optimized for expression in mammalian cells. In another embodiment, codon optimization increases the number of CpG motifs in the DNA prime.

Several methods can be used to optimize expression of the disulfide stabilized protein in vivo. For example, standard PCR cloning techniques could be used to insert into pVAX1 certain elements of the optimized PPI4 expression vector, including Intron A and adjoining regions of the CMV promoter. In addition, the genomic DNA sequences of the HIV-1 envelope are biased towards codons that are suboptimal for expression in mammalian cells (Haas, 1996). These can be changed to more favorable codons using standard mutagenesis techniques in order to improve the immunogenicity of nucleic acid-based HIV vaccines (Andre, 1998). The codon optimization strategy could also strive to increase the number of CpG motifs, which are known to increase the immunogenicity of DNA vaccines (Klinman, 1997). Lastly, as for the transient transfection systems described herein, Env processing into gp120-gp41 may be facilitated by the heterologous expression of furin introduced on the same or separate expression vectors.

A further embodiment of the instant method further comprises administering at least one adjuvant in combination with the DNA prime. In another embodiment, the adjuvant is a PLG particle or a CpG dinucleotide.

Adjuvants are formulations and/or additives that are routinely combined with antigens to boost immune responses. Suitable adjuvants for nucleic acid based vaccines include, but are not limited to, Quil A, imiquimod, resiquimod, interleukin-12 delivered in purified protein or nucleic acid form, short bacterial immunostimulatory nucleotide sequences such as CpG-containing motifs, interleukin-2/Ig fusion proteins delivered in purified protein or nucleic acid form, oil in water micro-emulsions such as MF59, polymeric microparticles, cationic liposomes, monophosphoryl lipid A, immunomodulators such as Ubenimex, and genetically detoxified toxins such as E. coli heat labile toxin and cholera toxin from Vibrio. Such adjuvants and methods of combining adjuvants with antigens are well known to those skilled in the art.

Adjuvants suitable for use with protein immunization include, but are not limited to, alum; Freund's incomplete adjuvant (FIA); saponin; Quil A; QS-21; Ribi Detox; monophosphoryl lipid A (MPL) adjuvants such as Enhanzyn™; nonionic block copolymers such as L-121 (Pluronic; Syntex SAF); TiterMax Classic adjuvant (block copolymer, CRL89-41, squalene and microparticulate stabilizer; Sigma-Aldrich); TiterMax Gold Adjuvant (new block copolymer, CRL-8300, squalene and a sorbitan monooleate; Sigma-Aldrich); Ribi adjuvant system using one or more of the following: monophosphoryl lipid A, synthetic trehalose, dicorynomycolate, mycobacterial cell wall skeleton incorporated into squalene and polysorbate-80; Corixa); RC-552 (a small molecule synthetic adjuvant; Corixa); Montanide adjuvants (including Montanide IMS111X, Montanide IMS131x, Montanide IMS221x, Montanide IMS301x, Montanide ISA 26A, Montanide ISA206,Montanide ISA 207, Montanide ISA25, Montanide ISA27, Montanide ISA28, Montanide ISA35, Montanide ISA50V, Montanide ISA563, Montanide ISA70, Montanide ISA 708, Montanide ISA740, Montanide ISA763A, and Montanide ISA773; Seppic Inc., Fairfield, N.J.); and N-Acetylmuramyl-L-alanyl-D-isoglutamine hydrate (Sigma-Aldrich). Methods of combining adjuvants with antigens are well known to those skilled in the art.

Because current vaccines depend on generating antibody responses to injected antigens, commercially available adjuvants have been developed largely to enhance these antibody responses. To date, the only FDA-approved adjuvant for use with human vaccines is alum. However, although alum helps boost antibody responses to vaccine antigens, it does not enhance T cell immune responses. Thus, there is wide interest in the development of new adjuvants that can be used to boost T cell immune responses after a vaccine is administered.

It is also known to those skilled in the art that cytotoxic T lymphocyte and other cellular immune responses are elicited when protein-based immunogens are formulated and administered with appropriate adjuvants, such as ISCOMs and micron-sized polymeric or metal oxide particles. Certain microbial products also act as adjuvants by activating macrophages, lymphocytes and other cells within the immune system, and thereby stimulating a cascade of cytokines that regulate immune responses. One such adjuvant is monophosphoryl lipid A (MPL) which is a derivative of the gram-negative bacterial lipid A molecule, one of the most potent immunostimulants known. The Enhanzyn™ adjuvant (Corixa Corporation, Hamilton, Mont.) consists of MPL, mycobacterial cell wall skeleton and squalene.

Adjuvants may be in particulate form. The antigen may be incorporated into biodegradable particles composed of poly-lactide-co-glycolide (PLG) or similar polymeric material. Such biodegradable particles are known to provide sustained release of the immunogen and thereby stimulate long-lasting immune responses to the immunogen. Other particulate adjuvants include, but are not limited to, micellular particles comprising Quillaia saponins, cholesterol and phospholipids known as immunostimulating complexes (ISCOMs; CSL Limited, Victoria AU), and superparamagnetic particles. Superparamagnetic microbeads include, but are not limited to, μMACS™ Protein G and μMACS™ Protein A microbeads (Miltenyi Biotec), Dynabeads® Protein G and Dynabeads® Protein A (Dynal Biotech). In addition to their adjuvant effect, superparamagnetic particles such as μMACS™ Protein G and Dynabeads® Protein G have the important advantage of enabling immunopurification of proteins.

In one embodiment of the first method, the nucleic acid prime is administered by intramuscular injection. In a further embodiment, the nucleic acid prime is administered by in vivo electroporation.

Electroporation works on the principle that a cell acts as an electrical capacitor and is generally unable to transmit an electrical current. Subjecting cells to a high-voltage electric field is thought, therefore, to create transient microscopic pores in the cell membrane. These pores are large enough to allow macromolecules such as nucleic acids and proteins, as well as pharmaceutical drugs and other polar compounds, to gain access to the interior of the cell. With time, the pores in the cell membrane close and the cell once again becomes impermeable. It has been demonstrated that electroporation can be applied in vivo to deliver nucleic acids and proteins into cells residing within skeletal muscle of live animals including rats, mice and rabbits (see U.S. Pat. Nos. 6,110,161; 6,262,281; and 6,610,044). The method involves first injecting the nucleic acid or protein into the muscle at one or multiple sites. Immediately or shortly after injection, electrodes are then placed flanking the injection site(s) and a specific amount of electrical current is passed through the muscle. The electrical current makes the muscle permeable, thus allowing the nucleic acid or protein to enter the cell. The efficiency of delivery of DNA into muscle cells permits robust immune responses using DNA vaccines and produces sufficient secreted proteins for systemic biological activity to be observed (U.S. Pat. Nos. 6,110,161; 6,262, 281; and 6,610,044).

In another embodiment, the protein boost composition is administered by intramuscular injection. Yet another embodiment comprises administering at least one adjuvant in combination with the protein boost composition. In a further embodiment, the at least one adjuvant is a saponin or a monophosphoryl lipid A (MPL) adjuvant. In a still further embodiment, the saponin adjuvant is QS-21. In an additional embodiment, the monophosphoryl lipid A (MPL) adjuvant comprises mycobacterial cell wall skeleton and squalene.

In a further embodiment, a first and a second adjuvant are administered sequentially in multiple boosts. In a still further embodiment, the first adjuvant is QS-21 and the second adjuvant comprises monophosphoryl lipid A (MPL) adjuvant, mycobacterial cell wall skeleton and squalene. In another embodiment, the first adjuvant is administered in combination with at least four boosts and the second adjuvant is administered in combination with at least two boosts.

This invention also provides a method for preventing a subject from becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-1. In one embodiment of this method, the subject has been exposed to HIV-1.

This invention further provides a method for reducing the likelihood of a subject becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-1. In one embodiment, the subject has been exposed to HIV-1.

This invention still further provides a method for preventing or delaying the onset of, or slowing the rate of progression of, an HIV-1-related disease in an HIV-1-infected subject which comprises administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

In one aspect of the invention, immunization is to be performed in a manner that biases the immune system in a preferred direction, for example, in the direction of a preferred T helper 1 type of immune response or a more T helper 2 type of immune response. It is now widely accepted that T cell-dependent immune responses can be classified on the basis of preferential activation and proliferation of two distinct subsets of CD4⁺ T-cells termed T_{H}1 and T_{H}2. These subsets can be distinguished from each other by restricted cytokine secretion profiles. The T_{H}1 subset is a high producer of IFN-γ with limited or no production of IL-4, whereas the T_{H}2 phenotype typically shows high level production of both IL-4 and IL-5 with no substantial production of IFN-γ. Both phenotypes can develop from naive CD4⁺ T cells and at present there is much evidence indicating that IL-12 and IFN-γ on the one hand and IL-4 on the other are key stimulatory cytokines in the differentiation process of pluripotent T_{H}O precursor cells into T_{H}1 or T_{H}2 effector cells, respectively, in vitro and in vivo. Since IFN-γ inhibits the expansion and function of T_{H}2 effector cells and IL-4 has the opposite effect, the preferential expansion of either IFN-γ producing cells (pc) or IL-4 pc is indicative of whether an immune response mounts into a T_{H}1 or T_{H}2 direction. The cytokine environment, however, is not the only factor driving T_{H} lineage differentiation. Genetic background, antigen dose, route of antigen administration, type of antigen presenting cell (APC) and signaling via TCR and accessory molecules on T cells also play a role in differentiation.

In one aspect of the invention, the immune system is directed toward a more T helper 1 or 2 type of immune response through using vaccine compositions with the property of modulating an immune response in one direction or the other. In a preferred aspect of the invention at least part of said adjuvant function comprises means for directing the immune system toward a more T helper 1 or 2 type of immune response.

In another embodiment, the biasing is accomplished using vectors with the property of modulating an immune response in one direction or the other. Examples of vectors with the capacity to stimulate either a more T helper 1 or a more T helper 2 type of immune response or of delivery routes such as intramuscular or epidermal delivery can be found in Robinson (1997), Sjolander et al. (1997), Doe et al. (1996), Feltquate et al. (1997), Pertmer et al. (1996), Prayaga et al. (1997) and Raz et al. (1996).

In another aspect of the invention, the immune system is induced to produce innate immune responses with adjuvant potential in the ability to induce local inflammatory responses. These responses include interferons, B-chemokines, and chemokines in general, capable of attracting antigen processing and presenting cells as well as certain lymphocyte populations for the production of additional specific immune responses. These innate type responses have different characteristics depending on the vector or DNA used and their specific immunomodulating characteristics, including those encoded by CpG motifs, and as such, the site of immunization. By using in a specific sequence vaccine compositions containing at least one common specific vaccine antigen, different kinds of desired protective vaccine responses may be generated and optimized. Different kinds of desired immune responses may also be obtained by combining different vaccine compositions and delivering them at different or the same specific sites depends on the desired vaccine effect at a particular site of entry (i.e., oral, nasal, enteric or urogenital) of the specific infectious agent.

In one aspect, the instant vaccine comprises antigen-presenting cells. Antigen-presenting cells include, but are not limited to, dendritic cells, Langerhan cell, monocytes, macrophages, muscle cells and the like. Preferably said antigen presenting cells are dendritic cells. Preferably, said antigen presenting cells present said antigen, or an immunogenic part thereof, such as a peptide, or derivative and/or analogue thereof, in the context of major histocompatibility complex I or complex II.

The potential exists not only to substantially boost immune responses to the recombinant antigen, but to tailor the nature of the immune responses by priming and then delivering one or more subsequent boosts with different forms of the antigen or by delivering the antigen to different immunological sites and/or antigen-presenting cell populations. Indeed, the ability to induce preferred type-1 or type-2 like T-helper responses or to additionally generate specific responses at mucosal and/or systemic sites are envisioned with such an approach. Such protocols, also known as “prime-boost” protocols, are described in U.S. Pat. No. 6,210,663 B1 and PCT International Application Publication No. WO 00/44410.

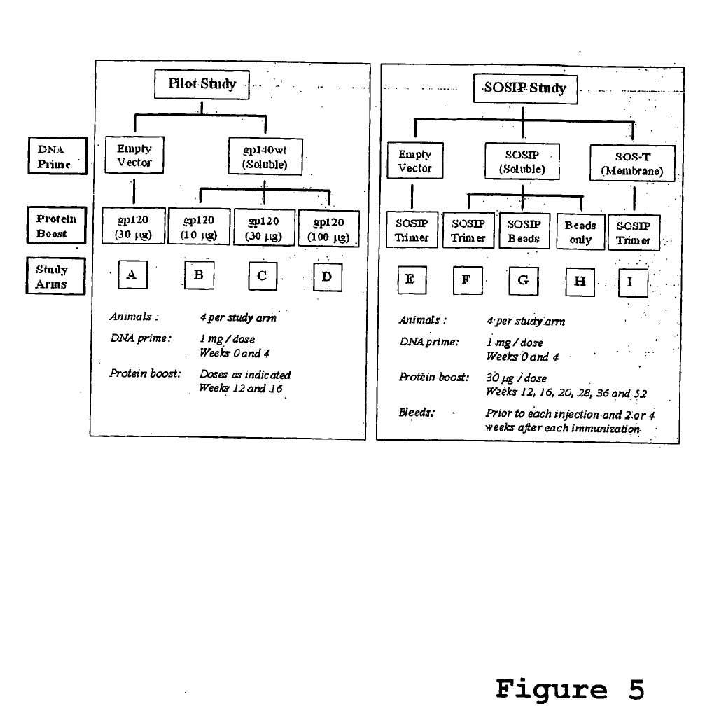

The designs of the prime-boost regimens used in the studies described herein are shown in FIG. 5. However, many variations of prime-boost protocols are possible, examples of which are listed in Table 1.

In one embodiment, vaccination is provided with at least three different vaccine compositions, wherein the vaccine compositions differ from each other by the form of the vaccine antigen.

TABLE 1
Examples of Prime-Boost Protocols

| Priming Composition | Boosting Composition |
|---|---|
| NA | AG |
| NA | AGP |
| NA | AG + AGP |
| AG | NA |
| AGP | NA |
| AG + AGP | NA |
| NA + AG | AGP |
| NA + AG | AG + AGP |
| NA + AG | AGP + NA |
| NA + AG + AGP | NA |
| NA + AG + AGP | NA + AG |
| NA + AG + AGP | NA + AGP |
| NA + AG + AGP | AG |
| NA + AG + AGP | AGP |
| NA + AG + AGP | AG + AGP |
| AG | NA |
| AGP | NA |
| AG + AGP | NA |
| AGP | NA + AG |
| AG + AGP | NA + AG |
| AGP + NA | NA + AG |
| NA | NA + AG + AGP |
| NA + AG | NA + AG + AGP |
| NA + AGP | NA + AG + AGP |
| AG | NA + AG + AGP |
| AGP | NA + AG + AGP |
| AG + AGP | NA + AG + AGP |
| AG | AGP |
| AG + AGP | AGP |
| AGP | AG |
| AGP | AG + AGP |

NA = Nucleic acid*
AG = Antigen
AGP = Particle-bound antigen
*The nucleic acid component in the above examples can be in the form of a viral vector component. The viral vector can be replicating or non-replicating.

For example, one embodiment of a priming vaccine composition is a replication-competent or replication-defective recombinant virus containing a nucleic acid molecule encoding the antigen, or a viral-like particle. In one particular embodiment, the priming composition is a non-replicating recombinant virus or viral-like particle derived from an α-virus.

One method according to this invention involves “priming” a mammalian subject by administration of a priming vaccine composition. In one embodiment, the priming vaccine, as with other instant compositions, is administered systemically. This systemic administration includes, for example, any parenteral route of administration characterized by physical breaching of a tissue of a subject and administration of an agent through the breach in the tissue. In particular, parenteral administration is contemplated to include, but is not limited to, intradermal, transdermal, subcutaneous, intraperitoneal, intravenous, intraarterial, intramuscular and intrasternal injection, intravenous, interaarterial and kidney dialytic infusion techniques, and so-called “needleless” injections through tissue. Preferably, the systemic, parenteral administration is intramuscular injection. In another embodiment, the instant vaccine is administered at a site of administration including the intranasal, oral, vaginal, intratracheal, intestinal and rectal mucosal surfaces.

The priming vaccine, as with other instant compositions, may be administered at various sites in the body in a dose-dependent manner. The invention is not limited to the amount or sites of administration(s) or to the pharmaceutical carrier, nor to this immunization protocol. Rather, the priming step encompasses treatment regimens which include a single dose or dosage which is administered hourly, daily, weekly, monthly, or yearly.

Preferably, a boosting vaccine composition is administered about 2 to 56 weeks after administering the priming vaccine to a mammalian subject. The administration of the boosting vaccine is accomplished using an effective amount of a boosting vaccine containing or capable of delivering the same antigen as administered by the priming vaccine.

In another example, one embodiment of a boosting vaccine composition is a replication-competent or replication-defective recombinant virus containing the DNA sequence encoding the protein antigen. In another embodiment, the boosting vaccine is a non-replicating α-virus comprising a nucleic acid molecule encoding the protein antigen or a non-replicating vaccine replicon particle derived from an Alphavirus. Adenoviruses, which naturally invade their host through the airways, infect cells of the airways readily upon intranasal application and induce a strong immune response without the need for adjuvants. In another embodiment, the boosting vaccine comprises a replication-defective recombinant adenovirus.

Another example of a boosting vaccine is a bacterial recombinant vector containing the DNA sequence encoding the antigen in operable association with regulatory sequences directing expression of the antigen in tissues of the mammal. One example is a recombinant BCG vector. Other examples include recombinant bacterial vectors based on Salmonella, Shigella, and Listeria, among others.

Still another example of a boosting vaccine is a naked DNA sequence encoding the antigen in operable association with regulatory sequences directing expression of the antigen in tissues of the mammal but containing no additional vector sequences. These vaccines may further contain pharmaceutically suitable or physiologically acceptable carriers.

In still additional embodiments, the boosting vaccines can include proteins or peptides (intact and denatured), heat-killed recombinant vaccines, inactivated whole microorganisms, antigen-presenting cells pulsed with the instant proteins or infected/transfected with a nucleic acid molecule encoding same, and the like, all with or without adjuvants, chemokines and/or cytokines.

Cytokines that may be used in the prime and/or boost vaccine or administered separately from the prime and/or boost vaccine include, but are not limited, to interleukin-4, interleukin-5, interleukin-2, interleukin-12, interleukin-15, interleukin-18, GM-CSF, and combinations thereof. The cytokine may be provided by a vector expressing one or more cytokines.

Representative forms of antigens include a “naked” DNA plasmid, a “naked” RNA molecule, a DNA molecule packaged into a replicating or nonreplicating viral vector, an RNA molecule packaged into a replicating or nonreplicating viral vector, a DNA molecule packaged into a bacterial vector, or proteinaceous forms of the antigen alone or present in virus-like particles (VLPs), or combinations thereof. In one embodiment, VLPs contain the instant trimeric Env complex and a structural protein, such as HIV-1 gag, needed to form membrane-enveloped virus-like particles.

Advantages of VLPs include (1) their particulate and multivalent nature, which is immunostimulatory, and (2) their ability to present the disulfide-stabilized envelope glycoproteins in a near-native, membrane-associated form.

VLPs are produced by co-expressing the viral proteins (e.g., HIV-1 gp120/gp41 and gag) in the same cell. This can be achieved by any of several means of heterologous gene expression that are well-known to those skilled in the art, such as transfection of appropriate expression vector(s) encoding the viral proteins, infection of cells with one or more recombinant viruses (e.g., vaccinia) that encode the VLP proteins, or retroviral transduction of the cells. A combination of such approaches can also be used. The VLPs can be produced either in vitro or in vivo.

VLPs can be produced in purified form by methods that are well-known to the skilled artisan, including centrifugation, as on sucrose or other layering substance, and by chromatography.

In one embodiment the instant nucleic acid delivery vehicle replicates in a cell of an animal or human being vaccinated. In one embodiment, said replicating nucleic acid has as least a limited capacity to spread to other cells of the host and start a new cycle of replication and antigen presentation and/or perform an adjuvant function. In another embodiment, the nucleic acid is non-replicating in an animal or human being being vaccinated. The nucleic acid can comprise nucleic acid of a poxvirus, a Herpes virus, a lentivirus, an Adenovirus, or adeno-associated virus. In a preferred embodiment, the nucleic acid comprises nucleic acid of an α-virus including, but not limited to, Venezuelan equine encephalitis (VEE) virus, Semliki Forest Virus, Sindbis virus, and the like. In another embodiment, said nucleic acid delivery vehicle is a VEE virus particle, Semliki Forest Virus particle, a Sindbis virus particle, a pox virus particle, a herpes virus particle, a lentivirus particle, or an adenovirus particle.

The instant vaccine can comprise, but is not limited to, the following: a recombinant subunit protein, a DNA plasmid, an RNA molecule, a replicating viral vector, a non-replicating viral vector, or a combination thereof.

The present invention also provides a second method for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a superparamagnetic particle and a trimeric protein complex so affixed thereto as to permit recognition of the complex by a subject's immune system, wherein in the trimeric complex each monomeric polypeptide unit comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41. In one embodiment, the trimeric protein complex is affixed to the superparamagnetic particle monovalently via one of three available epitopes in the complex, so as to leave two epitopes accessible to permit recognition of the complex by a subject's immune system. In another embodiment of this method, the trimeric protein complex is affixed to the superparamagnetic particle monovalently via one of three available epitopes in the complex, so as to leave two epitopes accessible to permit recognition of the complex by a subject's immune system.

A wide variety of antibodies that are known to bind to gp120 or gp41 epitopes may be used as agents to affix the subject trimeric protein complex to a superparamagnetic particle. Examples include the human MAb 2G12 (National Institutes of Health AIDS Research and Reference Reagent Program [ARRRP] Cat. No. 1476) which binds a C3-V4 glycan-dependent epitope on gp120 (Trkola et al., 1996); the human MAb 2F5 (ARRRP Cat. No. 1475) binds a cluster 1 neutralizing epitope centered on the sequence ELDKWA in gp41 (Muster et al., 1993; Parker et al., 2001); and the murine MAb PA1 binds a V3 loop epitope. Additional examples of antibodies which may be used include, but are not limited to, those listed in the Materials section of the Experimental Details.

The superparamagnetic particles used in the experiments described herein were μMACS™ Protein G microbeads (Miltenyi Biotec, Auburn, Calif.). However, one skilled in the art would recognize that a variety of other superparamagnetic particles may be used. These include, but are not limited to, μMACS™ Protein A microbeads (Miltenyi Biotec), Dynabeads® Protein G and Dynabeads® Protein A (Dynal Biotech, Brown Deer, Wis.). These superparamagnetic particles are small particles (mean diameters typically <10 μm) that exhibit magnetic properties when placed in a magnetic field, but have no residual magnetism when removed from the magnetic field. Accordingly, molecules or cells affixed to the surface of these particles are only subjected to magnetism during the time of isolation with a magnet. Thus, the particles do not aggregate but remain evenly dispersed in suspension. As described hereinbelow, superparamagnetic particles may be used as particulate adjuvants. They have the added advantage that they facilitate purification of antibodies or antibody-bound proteins. For example, Protein A or Protein G moieties attached to the surface of the particles may be used to bind antibodies via the Fc region, and specific antibodies thus affixed to the particles may be used to bind targeted proteins. The magnetically labeled antibody or protein can then be easily isolated using a magnet. Furthermore, superparamagnetic particles are also commercially available pre-coated with a variety of antibodies and ligands other than Protein G or protein A.

Alternatively, uncoated particles are available with a variety of surface chemistries that permit flexibility in the attachment of different types of ligands to the surface of the particles.

In another embodiment of the instant method, the modified gp41 comprises an I559P substitution. In yet another embodiment, the modified gp120 comprises an A501C mutation. In a further embodiment, the modified gp41 comprises a T605C mutation. In a still further embodiment, the modified gp120 comprises an A501C mutation and the modified gp120 is bound to the modified gp41 by a disulfide bond between the cysteine residue at position 501 of the modified gp120 and the cysteine residue at position 605 of the modified gp41.

In yet another embodiment, the protein boost further comprises a pharmaceutically acceptable carrier.

In one embodiment of the present method, the first predefined interval is at least one week. In another embodiment, the first predefined interval is at least four weeks. In a further embodiment, the second predefined interval is the same as the first predefined interval. In a still further embodiment, the second predefined interval is at least one week. In yet another embodiment, the second predefined interval is at least four weeks. In a preferred embodiment, two doses of the nucleic acid prime are administered. In a further embodiment, the two doses of the nucleic acid prime are administered at week 0 and week 4. In a still further embodiment, six doses of the protein boost composition are administered at weeks 12, 16, 20, 28, 37 and 52.

Additionally, in one embodiment each dose of nucleic acid prime comprises 4 μg to 10 mg per kg body weight of the subject. In another embodiment, each dose of nucleic acid prime comprises 40 μg to 2.5 mg per kg body weight of the subject. In a further embodiment, each dose of nucleic acid prime comprises 150 pg to 1 mg per kg body weight of the subject. In a preferred embodiment, each dose of nucleic acid prime comprises about 400 pg per kg body weight of the subject.

In yet another embodiment, each dose of the protein boost composition comprises 0.1 μg to 1 mg per kg body weight of the subject. In a further embodiment, each dose of the protein boost composition comprises 1 to 250 μg per kg body weight of the subject. In a still further embodiment, each dose of the protein boost composition comprises 5 to 50 pg per kg body weight of the subject. In a preferred embodiment, each dose of the protein boost composition comprises about 12 pg per kg body weight of the subject.

In an additional embodiment of the subject method, the nucleic acid prime is a DNA prime. In another embodiment, the DNA prime encodes wild-type gp140. In a further embodiment, the DNA prime encodes cleaved HIV-1 gp140. In a still further embodiment, the DNA prime encodes a cleavage-enhanced HIV-1 gp140. In yet another embodiment, the DNA prime encodes uncleaved HIV-1 gp140. In another embodiment, the DNA prime encodes a variant of HIV-1 gp140 comprising a gp41 component which comprises a transmembrane domain that is capable of binding to a biological membrane. In a further embodiment, the gp41 component contains three additional amino acids compared to unmodified gp41 and ends in the sequence V₆₆₆NRV₆₆₉. In an additional embodiment, the codons in the DNA prime have been optimized for expression in mammalian cells. In another embodiment, codon optimization increases the number of CpG motifs in the DNA prime.

A further embodiment of the instant method further comprises administering at least one adjuvant in combination with the DNA prime. In another embodiment, the adjuvant is a PLG particle or a CpG dinucleotide. In a further embodiment, the nucleic acid prime is administered by intramuscular injection. In a still further embodiment, the nucleic acid prime is administered by in vivo electroporation.

In another embodiment, the protein boost composition is administered by intramuscular injection. Yet another embodiment comprises administering at least one adjuvant in combination with the protein boost composition. In a further embodiment, the at least one adjuvant is a saponin or a monophosphoryl lipid A (MPL) adjuvant. In a still further embodiment, the saponin adjuvant is QS-21. In an additional embodiment, the monophosphoryl lipid A (MPL) adjuvant comprises mycobacterial cell wall skeleton and squalene.

In a further embodiment, a first and a second adjuvant are administered sequentially in multiple boosts. In a still further embodiment, the first adjuvant is a saponin such as QS-21 and the second adjuvant comprises monophosphoryl lipid A (MPL) adjuvant, mycobacterial cell wall skeleton and squalene. In another embodiment, the first adjuvant is administered in combination with at least four boosts and the second adjuvant is administered in combination with at least two boosts.

This invention also provides a method for preventing a subject from becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-1. In one embodiment, the subject has been exposed to HIV-1.

This invention further provides a method for reducing the likelihood of a subject becoming infected with HIV-1 comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-1. In one embodiment of this method, the subject has been exposed to HIV-1.

This invention still further provides a method for preventing or delaying the onset of, or slowing the rate of progression of, an HIV-1-related disease in an HIV-1-infected subject which comprises administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

In addition, this invention provides a use of a nucleic acid prime and a protein boost composition for the manufacture of separate coadministerable medicaments for use in a regimen for eliciting an immune response in a subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically or therapeutically effective dose of a trimeric protein complex, wherein each monomeric polypeptide unit in the complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a use of a nucleic acid prime and a protein boost composition for the manufacture of separate coadministerable medicaments for use in a regimen for preventing a subject from becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention further provides a use of a nucleic acid prime and a protein boost composition for the manufacture of separate coadministerable medicaments for use in a regimen for reducing the likelihood of a subject becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention still further provides a use of a nucleic acid prime and a protein boost composition for the manufacture of separate coadministerable medicaments for use in a regimen for preventing or delaying the onset of, or slowing the rate of progression of, an HIV-1-related disease in an HIV-1-infected subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a therapeutically effective dose of a composition comprising a trimeric protein complex, wherein each monomeric polypeptide unit in the complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention additionally provides a use of a nucleic acid prime and a protein boost composition for the manufacture of separate coadministerable medicaments for use in a regimen for eliciting an immune response in a subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically or therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention also provides a use of a nucleic acid prime and a protein boost composition for the manufacture of separate coadministerable medicaments for use in a regimen for preventing a subject from becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention further provides a use of a nucleic acid prime and a protein boost composition for the manufacture of separate coadministerable medicaments for use in a regimen for reducing the likelihood of a subject becoming infected with HIV-1, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

This invention still further provides a use of a nucleic acid prime and a protein boost composition for the manufacture of separate coadministerable medicaments for use in a regimen for preventing or delaying the onset of, or slowing the rate of progression of, an HIV-1-related disease in an HIV-1-infected subject, wherein in the regimen (i) more than one dose of the nucleic acid prime, and (ii) more than one dose of the protein boost composition, are to be administered, wherein each dose of the nucleic acid prime is to be administered to the subject at a first predefined interval and each dose of the protein boost composition is to be administered to the subject at a second predefined interval, and wherein the protein boost composition comprises a therapeutically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-1 gp120 and a modified HIV-1 gp41, and wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

Neutralizing antibodies elicited by the trimeric Env complex can also be administered to a subject in, for example, a passive immunization protocol. Such antibodies would preferably be humanized to prevent their immunogenicity in a human subject. One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody.

U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and PCT International Application Publication No. WO 90/07861 which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies.

### Experimental Details

The following Experimental Details are set forth to aid in an understanding of the invention, and are not intended, and should not be construed, to limit in any way the invention set forth in the claims which follow thereafter.

### Materials and Methods

Purified gp120 and CD4 Proteins

Recombinant HIV-1_{JR-FL} gp120 protein was produced in purified form from Chinese hamster ovary (CHO) cells stably transfected with the PPI4-tPA-gp120_{JR-FL} plasmid as previously described (U.S. Pat. Nos. 5,866,163 and 5,869,624). Soluble CD4 was purchased from Bartels Corporation (Issaquah, Wash.). CD4-IgG2 protein was produced in purified form as described from CHO cells stably co-transfected with CD4-IgG2HC-pRcCMV and CD4-kLC-pRcCMV (Allaway, 1995).

### Plasmids

pPPI4-derived eukaryotic expression vectors encoding SOS and uncleaved forms of HIV-1_{JR-FL} gp140 have been described previously (Binley, 2000a; Trkola et al., 1996; Sanders et al., 2000; Sanders et al., 2002a). The plasmid designated PPI4-tPA-gp120_{JR-FL} was deposited pursuant to, and in satisfaction of, the requirements of the Budapest Treaty on the International Recognition of the Deposit of Microorganisms for the Purposes of Patent Procedure (the “Budapest Treaty”) with the American Type Culture Collection (ATCC), 10801 University Blvd., Manassas, Va. 20110-2209 under ATCC Accession Number 75431. The plasmid was deposited with the ATCC on Mar. 12, 1993. This vector contains the cytomegalovirus major immediate-early (CMV/MIE) promoter/enhancer linked to the full-length HIV-1 envelope gene whose signal sequence was replaced with that derived from tissue plasminogen activator. In the vector, a stop codon has been placed at the gp120 C-terminus to prevent translation of gp41 sequences, which are present in the vector. The vector also contains an ampicillin resistance gene, an SV40 origin of replication and a DHFR gene whose transcription is driven by the β-globin promoter.

The furin gene (Thomas et al., 1988) was expressed from plasmid pcDNA3.1-Furin (Binley et al., 2000a).

The expression vectors designated CD4-IgG2HC-pRcCMV and CD4-kLC-pRcCMV were deposited pursuant to, and in satisfaction of, the requirements of the Budapest Treaty with the ATCC under ATCC Accession Nos. 75193 and 75104.

HIV-1 gp140 and gp120 Glycoproteins

The SOS gp140 protein contains cysteine substitutions at residues A501 in the C5 region of gp120 and T605 in gp41 (numbered by reference to Env proteins of strain HIV-1_{HXB2}) (Binley, 2000a; Sanders et al., 2000). In gp140UNC, the sequence KRRVVQREKRAV (SEQ ID NO: 3) at the junction between gp120 and gp41ECTO has been replaced with a hexameric LR motif to prevent scission of gp140 into gp120 and gp41_{ECTO} (Binley et al, 2000a). Plasmids encoding variable-loop-deleted forms of HIV-1_{JR-FL} SOS gp140 have been described (Sanders et al., 2000). In these constructs, the tripeptide GAG is used to replace V1 loop sequences (D133-K155) and V2 loop sequences (F159-1194), alone or in combination. The SOS gp140UNC protein contains the same cysteine substitutions that are present in SOS gp140, but the residues REKR at the gp120-gp41ECTO cleavage site have been replaced by the sequence IEGR, to prevent gp140 cleavage. The SOSIP gp140 protein also contains the same cysteine substitutions that are present in SOS gp140, and additionally contains a I559P substitution in the gp41 subunit (Sanders et al., 2002a).

The SOSIP.R6 gp140 construct expresses a soluble gp140 protein containing the same substitutions as SOSIP gp140 and additionally contains a hexa-arginine (R6) motif in place of the original cleavage site between gp120 and gp41ECTO that is designed to increase the extent of gp140 cleavage into its constituents (Binley et al., 2002). When expressed in cell culture, the SOSIP.R6 gp140 protein forms a mixture of monomers, dimers, trimers and higher molecular weight forms, with trimers usually predominating (Sanders et al., 2002a). The configurations adopted by SOSIP.R6 proteins upon in vivo expression are not known.

The SOS.R6(T) construct expresses a membrane-bound version of SOS gp140 with a minimal cytoplasmic tail. It contains the SOS disulfide bond and the cleavage-enhancing R6 substitution, but without the I559P mutation. SOS.R6(T) was made by first amplifying a Wt gp140Act fragment from the full length gp160 JR-FL template, essentially as described elsewhere (Herrera et al., 2003). The gp140Act fragment contains the gp41 transmembrane domain with a short, 3 amino acid cytoplasmic tail. The additional SOS and R6 modifications were then made as described previously (Binley et al., 2000a; Binley et al., 2002). Truncation of the cytoplasmic domain of gp41, as in the SOS.R6(T) protein, circumvents natural sequences that promote down-regulation of Env, including the membrane proximal Tyr-based endocytosis motif. Elimination of these sequences has been shown to increase the cell surface expression of Env in vitro and the magnitude of Env-induced immune responses in vivo (Boltmann et al., 2001; Ye et al., 2004).

To create stable cell lines that secrete full-length HIV-1_{JR-FL} SOS gp140 or ΔV1V2 SOS gp140, DXB-11 dihydrofolate reductase (dhfr)-negative CHO cells were co-transfected with pcDNA3.1-Furin and either pPPI4-SOS gp140 (Binley et al., 2000a) or pPPI4-ΔV1V2* SOS gp140 (Sanders et al., 2000), respectively, using the calcium phosphate precipitation method. Doubly transformed cells were selected by passaging the cells in nucleoside-free α-MEM media containing 10% fetal bovine serum (FBS), geneticin (Life Technologies, Rockville, Md.) and methotrexate (Sigma, St. Louis, Mo.). The cells were amplified for gp140 expression by stepwise increases in methotrexate concentration, as described elsewhere (Allaway et al., 1995). Clones were selected for SOS gp140 expression, assembly, and endoproteolytic processing based on SDS-PAGE and western blot analyses of culture supernatants. CHO cells expressing, SOS gp140UNC were created using similar methods, except that pcDNA3.1-Furin and geneticin were not used. Full-length SOS gp140 was purified from CHO cell culture supernatants by Galanthus nivalis lectin affinity chromatography (Sigma) and Superdex 200 gel filtration chromatography (Amersham Biosciences, Piscataway, N.J.), as described elsewhere (Trkola et al., 1996). The gp140UNC glycoprotein was purified by lectin chromatography only. The concentration of purified Env's was measured by UV spectroscopy as described (Scandella et al., 1993), and was corroborated by ELISA and densitometric analysis of SDS-PAGE gels. Recombinant HIV-1_{JR-FL}, HIV-1_{LAI} and HIV-1_{YU2} gp120 glycoproteins were produced using methods that have been previously described (Trkola et al., 1996; Wu et al., 1996).

Where indicated, HIV-1 envelope glycoproteins were transiently expressed in adherent 293T cells by transfection with Env- and furin-expressing plasmids, as described previously (Binley et al., 2000a). The SOSIP.R6 gp140 protein was purified from the concentrated supernatants by an initial fractionation with 50% ammonium sulfate. The supernatant solution was subsequently processed using Galanthus nivalis lectin affinity chromatography (Vector Laboratories, Burlingame, Calif.). The trimeric form of SOSIP.R6 gp140 was separated from other momomeric and oligomeric Env proteins by gel filtration chromatography on Superose 6 columns (Amersham Biosciences) equilibrated with phosphate-buffered saline (PBS). The eluate fractions containing the trimeric SOSIP.R6 gp140 protein were identified by Blue-Native PAGE analysis, then combined and concentrated. The concentration of purified Env proteins were measured by UV spectroscopy.

For radioimmunoprecipitation assays, the proteins were metabolically labeled with [³⁵S]cysteine and [³⁵S]methionine for 24 h prior to analysis.

### MAbs and CD4-Based Proteins

The epitopes for, and some immunochemical properties of, anti-HIV-1 Env MAbs from various donors used in the present study have been described previously (Moore, 1994a; Moore and Sodroski, 1996). These include: IgG1b12 and F91 [against the CD4 binding site (Burton et al., 1994; Moore and Sodroski, 1996)]; 2G12 [against a unique C3-V4 glycan-dependent epitope (Trkola et al., 1996)]; 17b [against a CD4-inducible epitope (Thali et al., 1993; Moore and Sodroski, 1996); 19b [against the V3 loop (Moore et al., 1995)]; 23A and D7324 [against the C5 region (Moore and Sodroski, 1996)]; 50.1 and 83.1 [against the V3 loop (White-Scharf et al., 1993)]; M90 [against the C1 region (diMarzo Veronese et al., 1992)]; 212A [against a conformational C1-C5 epitope (Moore et al., 1994b)]; A32 [against a CD4-inducible C1-C4 epitope (Moore and Sodroski, 1996; Sullivan et al., 1998); G3-519 and G3-299 [against C4 or C4/V3 epitopes (Moore and Sodroski, 1996)]. Anti-gp41 MAbs include: 2F5 [against a cluster 1 neutralizing epitope encompassing residues 665-690 and centered on the sequence ELDKWA (Muster et al., 1993; Parker et al., 2001)]; 7B2 [against epitope cluster 1 (kindly provided by Jim Robinson, Tulane University)]; 25C2 [against the fusion peptide region (Buchacher, 1994)]; and 2.2B [against epitope cluster II]. MAbs IgG1b12, 2G12 and 2F5 are broadly neutralizing (Trkola et al., 1995). MAb 17b weakly neutralizes diverse strains of HIV-1, more so in the presence of soluble CD4 (Thali et al., 1993), whereas the neutralizing activity of MAb 19b against primary isolates is limited (Trkola et al., 1998). MAbs 23A and 2.2B are non-neutralizing.

Soluble CD4 (sCD4) and the tetrameric CD4-based molecule CD4-IgG2 have been described elsewhere (Allaway, 1995).

Anti-HIV antibodies were obtained from commercial sources, from the NIH AIDS Reagent Program, or from the inventors.

Antibodies were biotinylated, where indicated, with NHS-biotin (Pierce, Rockford, Ill.) according to the manufacturer's instructions.

Expression of gp140s in transiently transfected 293T cells Recombinant HIV envelope proteins may be produced by a) transfecting a mammalian cell with an expression vector for producing mutant envelope glycoprotein; b) culturing the resulting transfected mammalian cell under conditions such that mutant envelope protein is expressed; and c) recovering the mutant envelope protein so expressed.

HIV envelope proteins were transiently expressed in adherent 293T cells, a human embryonic kidney cell line (ATCC Cat. Number CRL-1573) transfected with the SV40 large T antigen, which promotes high level replication of plasmids such as PPI4 that contain the SV40 origin. 293T cells were grown in Dulbecco's minimum essential medium (DMEM; Life Technologies, Gaithersburg, Md.) containing 10% fetal bovine serum supplemented with L-glutamine, penicillin, and streptomycin. Cells were plated in a 10-cm dish and transfected with 10 μg of purified pPPI4 plasmid using the calcium phosphate precipitation method. On the following day, cells were supplied fresh DMEM containing 0.2% bovine serum albumin along with L-glutamine, penicillin and streptomycin. In certain experiments, the cells were cotransfected with 10 μg of a pcDNA3.1 expression vector (Invitrogen, Carlsbad, Calif.) encoding the gene for human furin.

Various techniques may be employed to transfect or introduce recombinant expression vectors into an appropriate mammalian cell host. These include, for example, protoplast fusion, calcium phosphate precipitation, electroporation, retroviral transduction, or other conventional techniques. In the case of protoplast fusion, the cells are grown in media and screened for the appropriate activity. Expression of the gene encoding a mutant envelope protein results in production of the mutant protein.

Methods and conditions for culturing the resulting transfected cells and for recovering the mutant envelope protein so produced are well known to those skilled in the art, and may be varied or optimized depending upon the specific expression vector and mammalian host cell employed.

In accordance with the claimed invention, the preferred host cells for expressing the mutant envelope protein of this invention are mammalian cell lines. Mammalian cell lines include, for example, monkey kidney CV1 line transformed by SV40 (COS-7); human embryonic kidney line 293; baby hamster kidney cells (BHK); Chinese hamster ovary-cells-DHFR⁺ (CHO); Chinese hamster ovary-cells DHFR (DXB11); monkey kidney cells (CV1); African green monkey kidney cells (VERO-76); human cervical carcinoma cells (HELA); canine kidney cells (MDCK); human lung cells (W138); human liver cells (Hep G2); mouse mammary tumor (MMT 060562); mouse cell line (C127); and myeloma cell lines.

Other eukaryotic expression systems utilizing non-mammalian vector/cell line combinations can be used to produce the mutant envelope proteins. These include, but are not limited to, baculovirus vector/insect cell expression systems and yeast shuttle vector/yeast cell expression systems.

### ELISA Analyses

The concentration of gp120 and gp140 proteins in 293T cell supernatants was measured by ELISA (Binley et al., 1997b). Briefly, Immulon II ELISA plates (Dynatech Laboratories, Inc.) were coated for 16-20 h at 4EC with a polyclonal sheep antibody that recognizes the carboxy-terminal sequence of gp120 (APTKAKRRVVQREKR) (SEQ ID NO: 4). The plate was washed with Tris-buffered saline (TBS) and then blocked with 2% nonfat milk in TBS. Cell supernatants (100 μl) were added in a range of dilutions in TBS containing 10% fetal bovine serum. The plate was incubated for 1 h at ambient temperature and washed with TBS. Anti-gp120 or anti-gp41 antibody was then added for an additional 1 h. The plate was washed with TBS, and the amount of bound antibody was detected using alkaline phosphatase-conjugated goat anti-human IgG or goat anti-mouse IgG. Alternatively, biotinylated reporter antibodies are used according to the same procedure and detected using a streptavidin-AP conjugate. In either case, AP activity is measured using the AMPAK kit (DAKO) according to the manufacturer's instructions. To examine the reactivity of denatured HIV envelope proteins, the cell supernatants were boiled for 5 min in the presence of 1% of the detergents sodium dodecyl sulfate (SDS) and NP-40 prior to loading onto ELISA plates in a range of dilutions. Purified recombinant JR-FL gp120 was used as a reference standard.

### SDS-PAGE, Radioimmunoprecipitation, Blue Native PAGE, and Western Blot Analyses

Sodium dodecyl sulfate polyacrylamide gel electrophoresis (SDS-PAGE) analyses were performed as described elsewhere (Binley et al., 2000a). Reduced and non-reduced samples were prepared by boiling for 2 minutes in Laemmli sample buffer (62.5 mM Tris-HCl, pH 6.8, 2% SDS, 25% glycerol, 0.01% bromophenol blue) in the presence or absence, respectively, of 50 mM dithiothreitol (DTT). Protein purity was determined by densitometric analysis of the stained gels followed by the use of ImageQuant software (Molecular Devices, Sunnyvale, Calif.). Radioimmunoprecipitation assays (RIPA) were performed on Env-containing cell culture supernatants, as previously described (Binley et al., 2000a; Sanders et al., 2000).

Blue Native (BN)-PAGE was carried out with minor modifications to the published method (Schagger, 1994; Schagger, 1991). Thus, purified protein samples or cell culture supernatants were diluted with an equal volume of a buffer containing 100 mM 4-(N-morpholino)propane sulfonic acid (MOPS), 100 mM Tris-HCl, pH 7.7, 40% glycerol, 0.1% Coomassie blue, just prior to loading onto a 4-12% Bis-Tris NuPAGE gel (Invitrogen). Typically, gel electrophoresis was performed for 2 h at 150 V (˜0.07 A) using 50 mM MOPS, 50 mM Tris, pH 7.7, 0.002% Coomassie blue as cathode buffer, and 50 mM MOPS, 50 mM Tris, pH 7.7 as anode buffer. When purified proteins were analyzed, the gel was destained with several changes of 50 mM MOPS, 50 mM Tris, pH 7.7 subsequent to the electrophoresis step. Typically, 5 pg of purified protein were loaded per lane.

For western blot analyses, gels and polyvinylidine difluoride (PVDF) membranes were soaked for 10 minutes in transfer buffer (192 mM glycine, 25 mM Tris, 0.05% SDS, pH 8.8 containing 20% methanol). Following transfer, PVDF membranes were destained of Coomassie blue dye using 25% methanol and 10% acetic acid and air-dried. Destained membranes were probed using the anti-V3 loop MAb PA1 (Progenics) followed by horseradish peroxidase (HRP)-labeled anti-mouse IgG (Kirkegaard & Perry Laboratories, Gaithersburg, Md.), each used at 0.2 μg/ml final concentration. Luminometric detection of the envelope glycoproteins was obtained with the Renaissance7 Western Blot Chemiluminescence Reagent Plus system (Perkin Elmer Life Sciences, Boston, Mass.). Bovine serum albumin (BSA), apo-ferritin and thyroglobulin were obtained from Amersham Biosciences and used as molecular weight standards.

### Size Exclusion Chromatography

Purified, CHO cell-expressed SOS gp140, gp140UNC, SOSIP gp140 and gp120 proteins were analyzed by size exclusion chromatography on a TSK G3000SWXL HPLC column (TosoHaas, Montgomeryville, Pa.) using phosphate-buffered saline (PBS) as the running buffer. The protein retention time was determined by monitoring the UV absorbance of the column effluent at a wavelength of 280 nm. The column was calibrated using ferritin as a model protein that exists in oligomeric states of 220 kDa, 440 kDa and 880 kDa (Gerl et al., 1988).

### Preparation of 2G12 Microbeads

2.5 mg of the MAb, 2G12, were incubated with 20 ml of a suspension of μMACS™ Protein G microbeads (Miltenyi Biotec, Auburn, Calif.; Cat. No. 130-071-101) at 4° C. for 20 h. The microbeads were pelleted in a Sorvall RC5C centrifuge (SS-34 rotor) at 12,000 rpm (˜20,000×g) for 15 min. Unbound 2G12 was removed by washing with 400 μl of PBS and the pelleted 2G12-beads were resuspended in PBS at a concentration of ˜2 mg/ml (based on the 2G12 concentration).

The immobilization of CD4-IgG2, PA1, IgG1b12 and 2F5 was performed essentially as described for 2G12. The capacity of the microbeads was found to be ˜60 μg of 2G12, ˜40 μg of CD4-IgG2, ˜150 μg of PA1, ˜55 μg of IgG1b12, and ˜95 μg of 2F5 per ml of microbead suspension.

As is known to one skilled in the art, similar protocols can be used to capture Ig-containing proteins onto μMACS™ Protein A (Miltenyl Biotec), Dynabeads® Protein G (Dynal Biotech Inc., Brown deer, WI), or Dynabeads® Protein A (Dynal Biotech) microbeads.

### Purification of Trimeric SOSIP Protein

Supernatants from 293T cells transiently co-transfected with the SOSIP and furin plasmid were concentrated ˜100-fold, and the trimeric SOSIP protein was purified to homogeneity by ammonium sulfate fractionation, lectin and size exclusion chromatography.

### Preparation of SOSIP-2G12-Microbeads

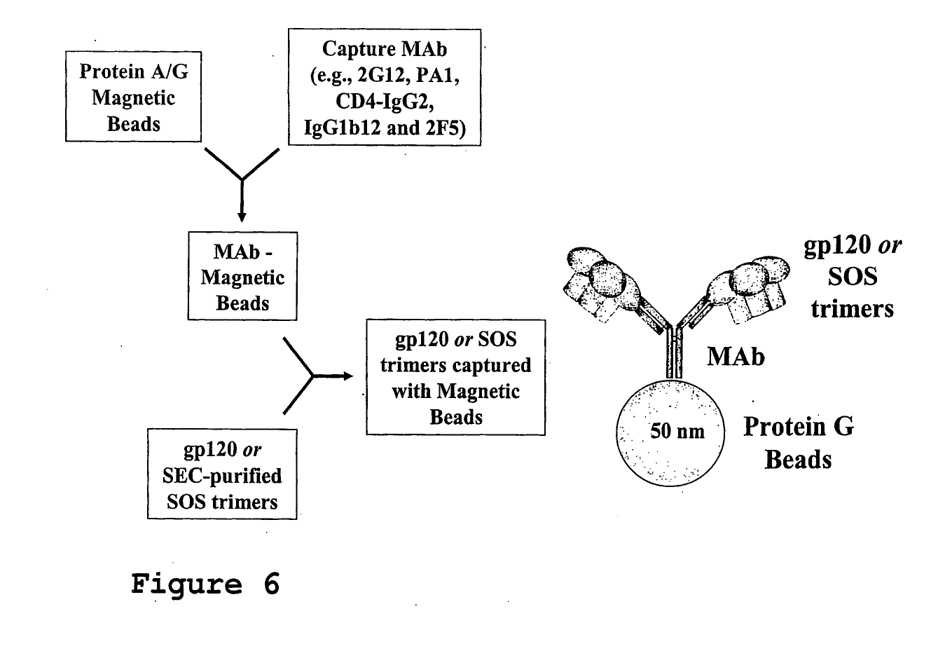

1 mg of purified trimeric SOSIP gp140 was incubated with 500 pg (250 μl) of 2G12-microbeads at 4° C. for 36 h. Following the capture of SOSIP gp140, the microbeads were pelleted in a Sorvall RC5C centrifuge (SS-34 rotor) at 12,000 rpm (˜20,000×g) for 15 min. The isolated microbeads were washed once with 400 μl PBS and pelleted in a microcentrifuge at 15,000 rpm (˜16,000×g) for 15 min. The washed SOSIP-2G12-microbeads were thoroughly resuspended with PBS at a concentration of ˜1 mg/ml (based on the SOSIP concentration). The beads were stored frozen at −80° C. until use. The preparation of antibody-beads and immobilization of various Env proteins thereon are shown in FIG. 6.

### Immunization Protocol Using Microbead Immunogens and a Heterologous Prime-Boost Regimen

Successful immunization relies on the induction of a protective immune response to an antigen of interest. Effective presentation of antigen to the immune system can be achieved by delivery of highly purified protein with an immunostimulatory adjuvant. A dual-purpose approach using superparamagnetic microbeads that (1) enables efficient purification of antigen for immunization, and (2) may have an adjuvant effect in animals, is described.

The care, maintenance and immunization of rabbits was carried out at the facilities of Aldevron LLC (Fargo, N. Dak.) under contract. The facility operates within full compliance of the Animal Welfare Act, abides by the principles outlined in the NIH guide for the Care and Use of Laboratory Animals, follows U.S. Department of Agriculture guidelines on the use of laboratory animals, and has IACUC approval for the electroporation and immunization procedures.

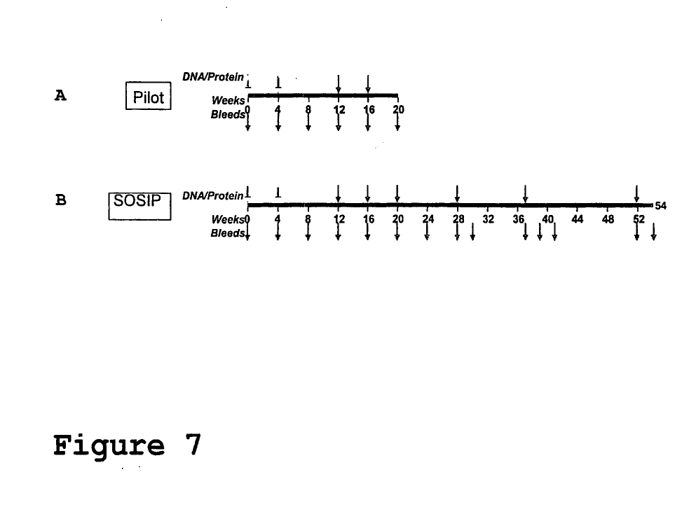

Each study arm consisted of 2 (Pilot study) or 4 animals (Second-stage SOSIP study). The immunization protocol was designed to use two DNA primes, followed by two (Pilot study) or 6 (Second-stage SOSIP study) protein boosts. The Pilot study examined the protein dose required to obtain maximum serum antibody titers in the rabbits. The Second-stage SOSIP study evaluated the immunogenicity of the SOSIP protein administered either as purified protein or coupled to superparamagnetic microbeads (Miltenyi Biotec, Auburn, Calif.). The designs of the Pilot study and the Second-stage SOSIP study are depicted in FIG. 5 and the timelines for the administration of the DNA and protein immunizations and the bleeds are shown in FIG. 7.

### Immunogens

For the DNA primes, plasmids encoding variant Env proteins were used as indicated (FIG. 5). For the protein boosts, purified monomeric gp120 (Subtype B, JR-FL; 1 mg/ml) or purified trimeric SOSIP gp140 was used at the indicated doses. The protein immunogens were admixed as indicated with the adjuvants, QS-21 (Antigenics Inc., Framingham, Mass.) or Enhanzyn™ (Corixa Corporation, Seattle, Wash.), and in some experiments captured on Miltenyi μMACS superparamagnetic particles by the anti-gp120 MAb, 2G12, as described above.

### DNA Prime

One year-old, female New Zealand Albino rabbits were anesthetized using an intramuscular (bicep) injection of a cocktail of ketamine, xylazine and acepromazine prior to the injection of the pPPI4-based, codon-optimized Env-expression plasmid into the hind limb skeletal muscle. A pre-immunization blood sample (8-10 ml) was taken by venipuncture prior to injection of the sterile filtered plasmid (1 mg in PBS, administered as two 0.5 mg doses at two sites) as outlined in the study design (FIG. 5). A mild current was then applied by a caliper-type electrode, allowing the uptake of DNA to be enhanced by “electroporation” (see U.S. Pat. Nos. 6,110,161; 6,262,281; and 6,610,044). The rabbits were DNA-immunized using this electroporation procedure at weeks 0 and 4, followed by several boosting immunizations with a JR-FL-based Env protein construct, formulated in QS-21 adjuvant.

The nucleic acid can also be administered to the animal by other methods, known to those skilled in the art, such as direct intramuscular injection or using gene gun techniques.

### Protein Boost

For the protein immunization, Env proteins were administered by injection into multiple anatomical sites (300 μl intramuscular, in each hind leg, 50 μl intradermal at six sites, and 100 μl subcutaneous in the neck region; a total of 1 ml), as described elsewhere (VanCott et al., 1997). In the Pilot study, each animal received either 10, 30, or 100 μg of purified protein per dose (approximately 4, 7.5 or 40 μg protein per kg body weight, respectively) on weeks 12 and 16 (see FIG. 7A). Each protein dose was adjuvanted with 100 μg of QS-21.

For the SOSIP study, each animal received 30 μg of purified trimeric SOSIP gp140 (tSOSIP), trimeric SOSIP-beads (tSOSIP-beads), or empty 2G12-beads per dose (approximately 7.5 μg protein per kg body weight) on weeks 12, 16, 20, 28, 37, and 52 (see FIG. 7B). Each dose also contained QS-21 as adjuvant throughout the entire protein boost part of the study, except that in the later stages of the SOS.R6(T)/SOSIP.R6 study group (Arm E), from week 37, Enhanzyn™ was used as an alternative adjuvant to QS-21.

Codon optimized constructs used for DNA primes in the immunization study (see FIG. 5) are shown in Sequences 1-3.

| Sequence 1. Codon optimized DNA sequence of wild-type JR-FL | |
|---|---|
| gp140 (gp140wt) | |
| DNA open reading frame: | |
| (SEQ ID NO: 5) | |
| atggatgcaatgaagagagggctctgctgtgtgctgctgctgtgtggagcagtcttcgtttcgcccagccag | |
| gaaatccatgcccgattcagaagaggcgccgctgtggagaagctgtgggtgactgtatactatggggtgcct | |
| gtgtggaaggaggccaccaccaccctgttctgtgcctctgatgccaaggcctatgacactgaggtccacaat | |
| gtctgggccacccatgcctgtgtgcccactgaccccaaccctcaggaggtggtgctggagaatgtgactgag | |
| cacttcaacatgtggaagaacaacatggtggagcagatgcaggaggacatcatcagcctgtgggaccagagc | |
| ctgaagccctgtgtgaagctgacccccctgtgtgtgaccctgaactgcaaggatgtgaatgccaccaacacc | |
| accaatgactctgagggcactatggagaggggtgagatcaagaactgcagcttcaacatcaccaccagcatc | |
| agggatgaggtgcagaaggagtatgccctgttctacaagctggatgtggtgcccattgacaacaacaacacc | |
| agctacaggctgatcagctgtgacacctctgtgatcacccaggcctgccccaagatcagctttgagcccatc | |
| cccatccactactgtgcccctgctggctttgccatcctgaagtgcaatgacaagaccttcaatggcaaaggc | |
| ccttgcaagaatgtgagcactgtgcagtgcactcatggcatcaggcctgtggtgagcacccagctgctgctg | |
| aatggcagcctggctgaggaggaggtggtgatcaggtctgacaacttcaccaacaatgccaagaccatcatt | |
| gtgcagctgaaggagtctgtggagatcaactgcaccaggcccaacaacaacaccaggaagagcattcacatt | |
| ggccctggcagggccttctacaccactggggagatcattggggacatcaggcaggcccactgcaacatcagc | |
| agggccaagtggaatgacaccctgaagcagattgtgatcaagctgagggagcagtttgagaacaagaccatt | |
| gtgttcaatcacagctctggtggtgatcctgagattgtgatgcacagcttcaactgtggtggtgagttcttc | |
| tactgcaacagcacccagctgttcaacagcacctggaacaacaacactgagggcagcaacaacactgagggc | |
| aacaccatcaccctgccttgcaggatcaagcagatcatcaacatgtggcaggaggtgggcaaggccatgtat | |
| gctcctcccatcaggggccagatcaggtgcagcagcaacatcactggcctgctgctgaccagggatggtggc | |
| atcaatgagaatggcactgagattttcaggcctggtggtggggacatgagggacaactggaggtctgagctg | |
| tacaagtacaaggtggtgaagattgagccccttggtgtggctcccaccaaggctaagcgcagggtggtgcag | |
| agggagaagcgcgctgtgggcattggggctgtgttcctgggcttccttggggctgctggcagcactatgggt | |
| gctgccagcatgaccctgactgtgcaggccaggctgctgctgtctggcattgtgcagcagcagaacaacctg | |
| ctgagggccattgaggctcaacagaggatgctccagctcactgtctggggcatcaagcagctccaggccagg | |
| gtgctggctgtggagaggtatcttggggatcagcagctccttggcatctggggctgctctggcaagctgatc | |
| tgcaccactgctgtgccctggaatgccagctggagcaacaagagcctggacaggatctggaacaacatgacc | |
| tggatggagtgggagagggagattgacaactacacctctgagatttacaccctgattgaggagagccagaac | |
| cagcaggagaagaatgagcaggagctgctggagctggacaagtgggccagcctgtggaactggtttgacatc | |
| accaagtggctgtggtactaa | |
| Protein Sequence: | |
| gp120 subunit: | |
| (SEQ ID NO: 6) | |
| 1 | MRVKGIRKSY QYLWKGGTLL LGILMICSAV EKLWVTVYYG VPVWKEATTT LFCASDAKAY |
| 61 | DTEVHNVWAT HACVPTDPNP QEVVLENVTE HFNMWKNNMV EQMQEDIISL WDQSLKPCVK |
| 121 | LTPLCVTLNC KDVNATNTTN DSEGTMERGE IKNCSFNITT SIRDEVQKEY ALFYKLDVVP |
| 181 | IDNNNTSYRL ISCDTSVITQ ACPKISFEPI PIHYCAPAGF AILKCNDKTF NGKGPCKNVS |
| 241 | TVQCTHGIRP VVSTQLLLNG SLAEEEVVIR SDNFTNNAKT IIVQLKESVE INCTRPNNNT |
| 301 | RKSIHIGPGR AFYTTGEIIG DIRQAHCNIS RAKWNDTLKQ IVIKLREQFE NKTIVFNHSS |
| 361 | GGDPEIVMHS FNCGGEFFYC NSTQLFNSTW NNNTEGSNNT EGNTITLPCR IKQIINMWQE |
| 421 | VGKAMYAPPI RGQIRCSSNI TGLLLTRDGG INENGTEIFR PGGGDMRDNW RSELYKYKVV |
| 481 | KIEPLGVAPT KAKRRVVQRE KR |
| gp41 subunit: | |
| (SEQ ID NO: 7) | |
| 503 | AVGIGAVFLG FLGAAGSTMG AASMTLTVQA RLLLSGIVQQ QNNLLRAIEA QQRMLQLTVW |
| 563 | GIKQLQARVL AVERYLGDQQ LLGIWGCSGK LICTTAVPWN ASWSNKSLDR IWNNMTWMEW |
| 623 | EREIDNYTSE IYTLIEESQN QQEKNEQELL ELDKWASLWN WFDITKWLWY |
| Sequence 2. Codon optimized DNA sequence of JR-FL cleavage- | |
| enhanced gp140 SOSIP R6 (SOSIP.R6) | |
| DNA open reading frame: | |
| (SEQ ID NO: 8) | |
| atggatgcaatgaagagagggctctgctgtgtgctgctgctgtgtggagcagtcttcgtttcgcccagccag | |
| gaaatccatgcccgattcagaagaggcgccgctgtggagaagctgtgggtgactgtatactatggggtgcct | |
| gtgtggaaggaggccaccaccaccctgttctgtgcctctgatgccaaggcctatgacactgaggtccacaat | |
| gtctgggccacccatgcctgtgtgcccactgaccccaaccctcaggaggtggtgctggagaatgtgactgag | |
| cacttcaacatgtggaagaacaacatggtggagcagatgcaggaggacatcatcagcctgtgggaccagagc | |
| ctgaagccctgtgtgaagctgacccccctgtgtgtgaccctgaactgcaaggatgtgaatgccaccaacacc | |
| accaatgactctgagggcactatggagaggggtgagatcaagaactgcagcttcaacatcaccaccagcatc | |
| agggatgaggtgcagaaggagtatgccctgttctacaagctggatgtggtgcccattgacaacaacaacacc | |
| agctacaggctgatcagctgtgacacctctgtgatcacccaggcctgccccaagatcagctttgagcccatc | |
| cccatccactactgtgcccctgctggctttgccatcctgaagtgcaatgacaagaccttcaatggcaaaggc | |
| ccttgcaagaatgtgagcactgtgcagtgcactcatggcatcaggcctgtggtgagcacccagctgctgctg | |
| aatggcagcctggctgaggaggaggtggtgatcaggtctgacaacttcaccaacaatgccaagaccatcatt | |
| gtgcagctgaaggagtctgtggagatcaactgcaccaggcccaacaacaacaccaggaagagcattcacatt | |
| ggccctggcagggccttctacaccactggggagatcattggggacatcaggcaggcccactgcaacatcagc | |
| agggccaagtggaatgacaccctgaagcagattgtgatcaagctgagggagcagtttgagaacaagaccatt | |
| gtgttcaatcacagctctggtggtgatcctgagattgtgatgcacagcttcaactgtggtggtgagttcttc | |
| tactgcaacagcacccagctgttcaacagcacctggaacaacaacactgagggcagcaacaacactgagggc | |
| aacaccatcaccctgccttgcaggatcaagcagatcatcaacatgtggcaggaggtgggcaaggccatgtat | |
| gctcctcccatcaggggccagatcaggtgcagcagcaacatcactggcctgctgctgaccagggatggtggc | |
| atcaatgagaatggcactgagattttcaggcctggtggtggggacatgagggacaactggaggtctgagctg | |
| tacaagtacaaggtggtgaagattgagccccttggtgtggctcccaccaagtgcaagcgcagggtggtgcag | |
| aggaggaggaggcgcagggctgtgggcattggggctgtgttcctgggcttccttggggctgctggcagcact | |
| atgggtgctgccagcatgaccctgactgtgcaggccaggctgctgctgtctggcattgtgcagcagcagaac | |
| aacctgctgagggcccccgaggctcaacagaggatgctccagctcactgtctggggcatcaagcagctccag | |
| gccagggtgctggctgtggagaggtatcttggggatcagcagctccttggcatctggggctgctctggcaag | |
| ctgatctgctgcactgctgtgccctggaatgccagctggagcaacaagagcctggacaggatctggaacaac | |
| atgacctggatggagtgggagagggagattgacaactacacctctgagatttacaccctgattgaggagagc | |
| cagaaccagcaggagaagaatgagcaggagctgctggagctggacaagtgggccagcctgtggaactggttt | |
| gacatcaccaagtggctgtggtactaa | |
| Protein Sequence: | |
| gp120 subunit: | |
| (SEQ ID NO: 9) | |
| 1 | MRVKGIRKSY QYLWKGGTLL LGILMICSAV EKLWVTVYYG VPVWKEATTT LFCASDAKAY |
| 61 | DTEVHNVWAT HACVPTDPNP QEVVLENVTE HFNMWKNNMV EQMQEDIISL WDQSLKPCVK |
| 121 | LTPLCVTLNC KDVNATNTTN DSEGTMERGE IKNCSFNITT SIRDEVQKEY ALFYKLDVVP |
| 181 | IDNNNTSYRL ISCDTSVITQ ACPKISFEPI PIHYCAPAGF AILKCNDKTF NGKGPCKNVS |
| 241 | TVQCTHGIRP VVSTQLLLNG SLAEEEVVIR SDNFTNNAKT IIVQLKESVE INCTRPNNNT |
| 301 | RKSIHIGPGR AFYTTGEIIG DIRQAHCNIS RAKWNDTLKQ IVIKLREQFE NKTIVFNHSS |
| 361 | GGDPEIVMHS FNCGGEFFYC NSTQLFNSTW NNNTEGSNNT EGNTITLPCR IKQIINMWQE |
| 421 | VGKAMYAPPI RGQIRCSSNI TGLLLTRDGG INENGTEIFR PGGGDMRDNW RSELYKYKVV |
| 481 | KIEPLGVAPT KCKRRVVQRR RRRR |
| gp41 subunit: | |
| (SEQ ID NO: 10) | |
| 505 | AVGIGAVFLG FLGAAGSTMG AASMTLTVQA RLLLSGIVQQ QNNLLRAPEA QQRMLQLTVW |
| 565 | GIKQLQARVL AVERYLGDQQ LLGIWGCSGK LICCTAVPWN ASWSNKSLDR IWNNMTWMEW |
| 625 | EREIDNYTSE IYTLIEESQN QQEKNEQELL ELDKWASLWN WFDITKWLWY |

Mutations introduced into wild type sequence are shown in bold, underlined font. These mutations are Ala492 (492, 501) mutated into Cys (SOS disulfide bond formation); Thr598 (596, 605) mutated into Cys (SOS disulfide bond formation); Ile552 (550, 559) mutated into Pro (stabilization of gp140 prefusion conformation; and REKR (499-502, 508-511) mutated into RRRRRR (cleavage enhancement). The numbers in parentheses denote the location of the amino acid residues in the above sequence: the first number corresponds to the position in the original JR-FL sequence (without R6 cleavage enhancement) whereas the second number corresponds to the position in the HXB2 sequence.

| Sequence 3. Codon optimized DNA sequence of JRFL gp140T SOS | |
|---|---|
| R6 (SOS.R6.T) | |
| DNA open reading frame: | |
| (SEQ ID NO: 11) | |
| atggatgcaatgaagagagggctctgctgtgtgctgctgctgtgtggagcagtcttcgtttcgcccagccag | |
| gaaatccatgcccgattcagaagaggcgccgctgtggagaagctgtgggtgactgtatactatggggtgcct | |
| gtgtggaaggaggccaccaccaccctgttctgtgcctctgatgccaaggcctatgacactgaggtccacaat | |
| gtctgggccacccatgcctgtgtgcccactgaccccaaccctcaggaggtggtgctggagaatgtgactgag | |
| cacttcaacatgtggaagaacaacatggtggagcagatgcaggaggacatcatcagcctgtgggaccagagc | |
| ctgaagccctgtgtgaagctgacccccctgtgtgtgaccctgaactgcaaggatgtgaatgccaccaacacc | |
| accaatgactctgagggcactatggagaggggtgagatcaagaactgcagcttcaacatcaccaccagcatc | |
| agggatgaggtgcagaaggagtatgccctgttctacaagctggatgtggtgcccattgacaacaacaacacc | |
| agctacaggctgatcagctgtgacacctctgtgatcacccaggcctgccccaagatcagctttgagcccatc | |
| cccatccactactgtgcccctgctggctttgccatcctgaagtgcaatgacaagaccttcaatggcaaaggc | |
| ccttgcaagaatgtgagcactgtgcagtgcactcatggcatcaggcctgtggtgagcacccagctgctgctg | |
| aatggcagcctggctgaggaggaggtggtgatcaggtctgacaacttcaccaacaatgccaagaccatcatt | |
| gtgcagctgaaggagtctgtggagatcaactgcaccaggcccaacaacaacaccaggaagagcattcacatt | |
| ggccctggcagggccttctacaccactggggagatcattggggacatcaggcaggcccactgcaacatcagc | |
| agggccaagtggaatgacaccctgaagcagattgtgatcaagctgagggagcagtttgagaacaagaccatt | |
| gtgttcaatcacagctctggtggtgatcctgagattgtgatgcacagcttcaactgtggtggtgagttcttc | |
| tactgcaacagcacccagctgttcaacagcacctggaacaacaacactgagggcagcaacaacactgagggc | |
| aacaccatcaccctgccttgcaggatcaagcagatcatcaacatgtggcaggaggtgggcaaggccatgtat | |
| gctcctcccatcaggggccagatcaggtgcagcagcaacatcactggcctgctgctgaccagggatggtggc | |
| atcaatgagaatggcactgagattttcaggcctggtggtggggacatgagggacaactggaggtctgagctg | |
| tacaagtacaaggtggtgaagattgagccccttggtgtggctcccaccaagtgcaagcgcagggtggtgcag | |
| aggaggaggaggcgcagggctgtgggcattggggctgtgttcctgggcttccttggggctgctggcagcact | |
| atgggtgctgccagcatgaccctgactgtgcaggccaggctgctgctgtctggcattgtgcagcagcagaac | |
| aacctgctgagggccattgaggctcaacagaggatgctccagctcactgtctggggcatcaagcagctccag | |
| gccagggtgctggctgtggagaggtatcttggggatcagcagctccttggcatctggggctgctctggcaag | |
| ctgatctgctgcactgctgtgccctggaatgccagctggagcaacaagagcctggacaggatctggaacaac | |
| atgacctggatggagtgggagagggagattgacaactacacctctgagatttacaccctgattgaggagagc | |
| cagaaccagcaggagaagaatgagcaggagctgctggagctggacaagtgggccagcctgtggaactggttt | |
| gacatcaccaagtggctgtggtacatcaagattttcatcatgattgttggtggcctggtgggcctgaggctg | |
| gtgttcactgtcctgagcattgtgaacagggtgtaa | |
| Protein Sequence: | |
| gp120 subunit: | |
| (SEQ ID NO: 12) | |
| 1 | MRVKGIRKSY QYLWKGGTLL LGILMICSAV EKLWVTVYYG VPVWKEATTT LFCASDAKAY |
| 61 | DTEVHNVWAT HACVPTDPNP QEVVLENVTE HFNMWKNNMV EQMQEDIISL WDQSLKPCVK |
| 121 | LTPLCVTLNC KDVNATNTTN DSEGTMERGE IKNCSFNITT SIRDEVQKEY ALFYKLDVVP |
| 181 | IDNNNTSYRL ISCDTSVITQ ACPKISFEPI PIHYCAPAGF AILKCNDKTF NGKGPCKNVS |
| 241 | TVQCTHGIRP VVSTQLLLNG SLAEEEVVIR SDNFTNNAKT IIVQLKESVE INCTRPNNNT |
| 301 | RKSIHIGPGR AFYTTGEIIG DIRQAHCNIS RAKWNDTLKQ IVIKLREQFE NKTIVFNHSS |
| 361 | GGDPEIVMHS FNCGGEFFYC NSTQLFNSTW NNNTEGSNNT EGNTITLPCR IKQIINMWQE |
| 421 | VGKAMYAPPI RGQIRCSSNI TGLLLTRDGG INENGTEIFR PGGGDMRDNW RSELYKYKVV |
| 481 | KIEPLGVAPT KCKRRVVQRR RRRR |
| gp41 subunit: | |
| (SEQ ID NO: 13) | |
| 505 | AVGIGAVFLG FLGAAGSTMG AASMTLTVQA RLLLSGIVQQ QNNLLRAIEA QQRMLQLTVW |
| 565 | GIKQLQARVL AVERYLGDQQ LLGIWGCSGK LICCTAVPWN ASWSNKSLDR IWNNMTWMEW |
| 625 | EREIDNYTSE IYTLIEESQN QQEKNEQELL ELDKWASLWN WFDITKWLWY IKIFIMIVGG |
| 685 | LVGLRLVFTV LSIVNRV |

Mutations introduced into wild type sequence are shown in bold, underlined font. These mutations are Ala492 (492, 501) mutated into Cys (SOS disulfide bond formation); Thr598 (596, 605) mutated into Cys (SOS disulfide bond formation); and REKR (499-502, 508-511) mutated into RRRRRR (cleavage enhancement). The numbers in parentheses denote the location of the amino acid residues in the above sequence: the first number corresponds to the position in the original JR-FL sequence (without R6 cleavage enhancement) whereas the second number corresponds to the position in the HXB2 sequence.

### Bleeds

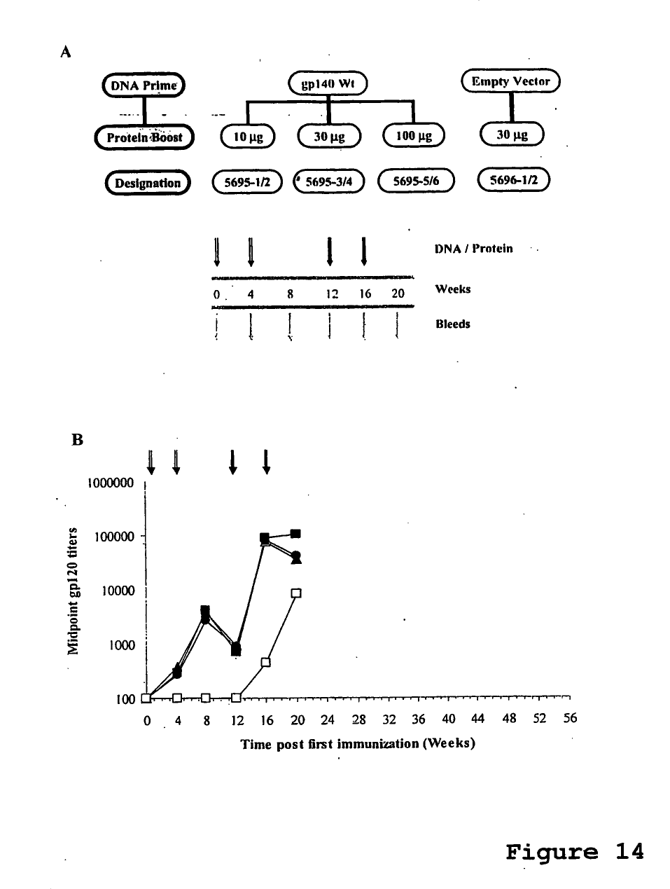

For the Pilot study, bleeds were taken from the animals prior to and 4 weeks after each immunization (see FIGS. 7A and 14A.

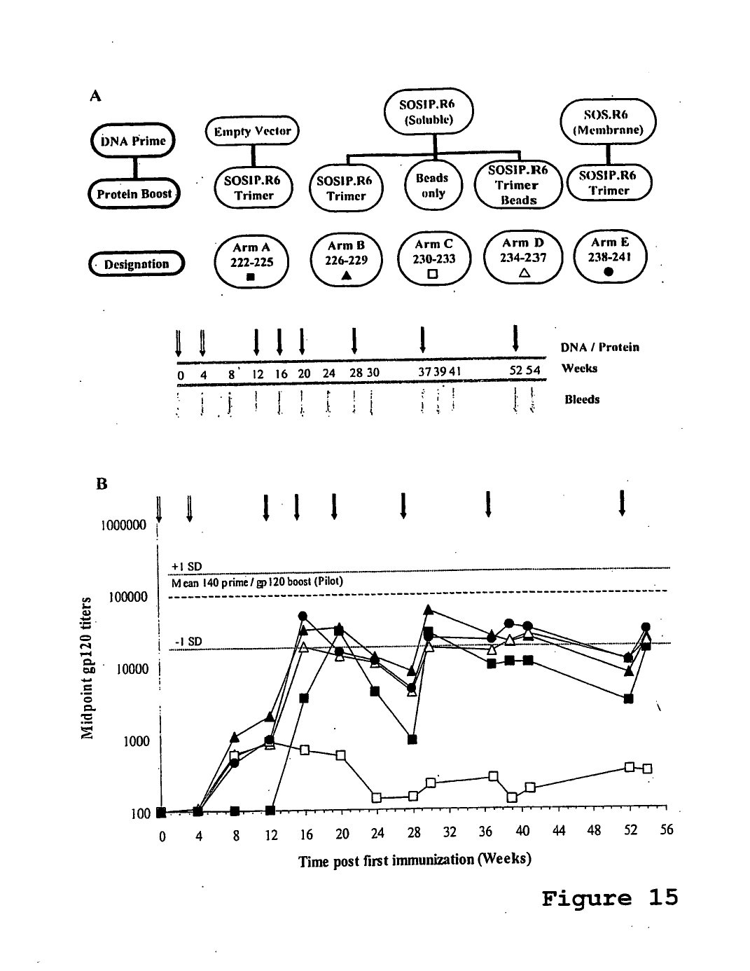

For the Second-stage SOSIP study, the timing of each bleed/dose and the constructs used for each study animal are outlined in FIGS. 7B and 15A.

Binding of Immune Sera to Monomeric gp120 and V3 Peptides

The cyclic peptides, V3_{JR-FL} (Sequence: Ac-CTRPNNNTRKSIHIGPGRAFYTTGEIIGDIRQAHC-NH₂; cyclized via a disulfide bond between the two terminal Cys residues) (SEQ ID NO: 14) and V3_{HXB2} (Sequence: Ac-CTRPNNNTRKRIRIQRGPGRAFVTIGKIGNMRQAHC-NH₂; cyclized via a disulfide bond between the two terminal Cys residues) (SEQ ID NO: 15), were synthesized by the American Peptide Company Inc., CA. PA1 is a V3_{JR-FL}-specific murine monoclonal antibody (MAb), as defined by its ability to bind the cyclic V3_{JR-FL} peptide, but not the cyclic V3_{HXB2} peptide or a V3-deleted gp120_{JR-FL} protein, in immunoassays. The CD4-immunoglobulin G2 (CD4-IgG2) molecule has been described elsewhere (Trkola et al., 1995). The binding of MAb, CD4-IgG2 and rabbit immune sera to monomeric gp120 was measured by enzyme-linked immunosorbent assay (ELISA). For assessment of antibody binding to V3 peptides, each peptide (10 μg/ml) was coated directly onto Immulon II plates overnight. The plates were then blocked with excess milk protein, then the assay was continued as described below for the monomeric gp120 ELISA. Midpoint binding titers were estimated by interpolation.

gp120 ELISA

gp120 was captured to microtiter plates via the sheep antibody 6205 (International Enzymes; Fairbrook, Calif.) and incubated with serial dilutions of the individual collected rabbit sera. Antibodies bound to gp120 were detected using the appropriate anti-species alkaline phosphatase conjugate IgG and the AMPAK calorimetric detection system (DakoCytomation; Carpinteria, Calif.) as previously described (Moore et al., 1996; Moore and Sodroski, 1996).

### SOSIP Elisa

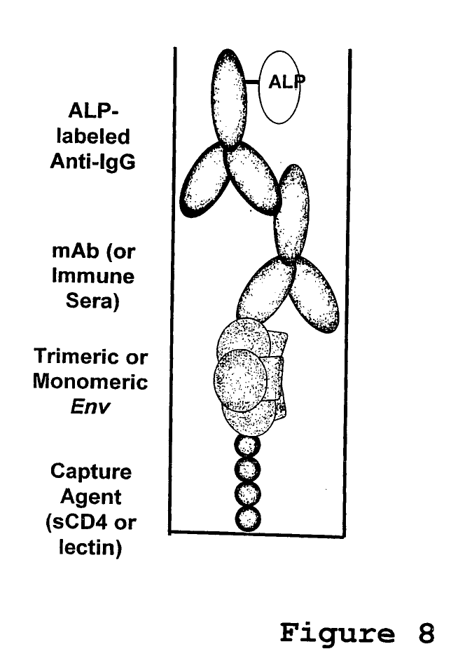

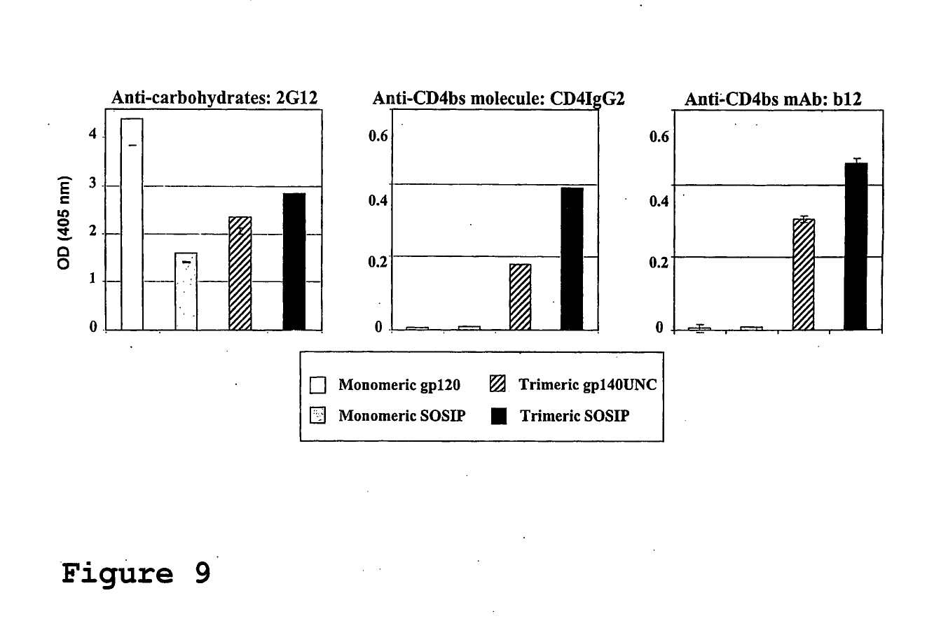

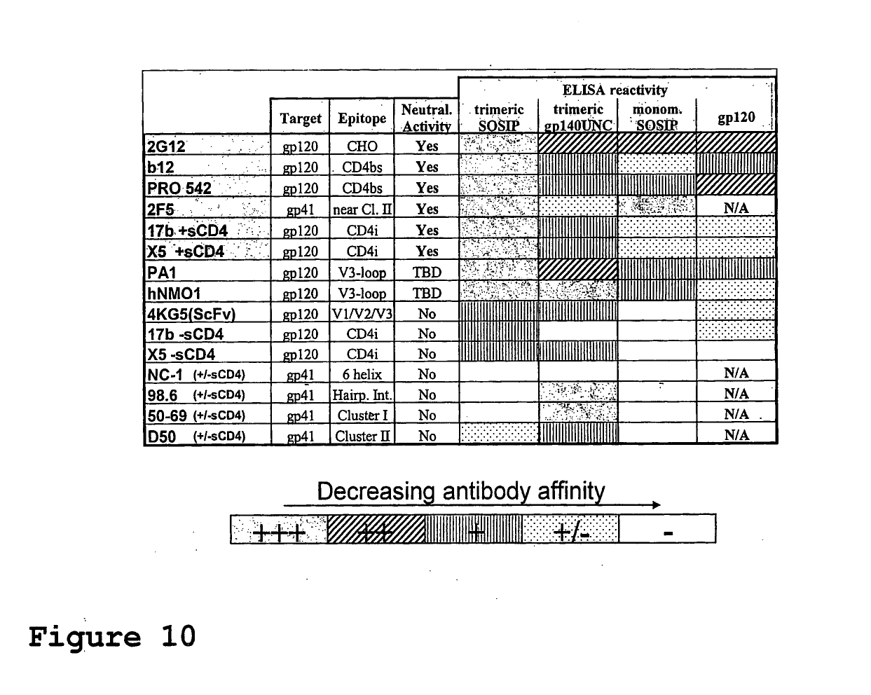

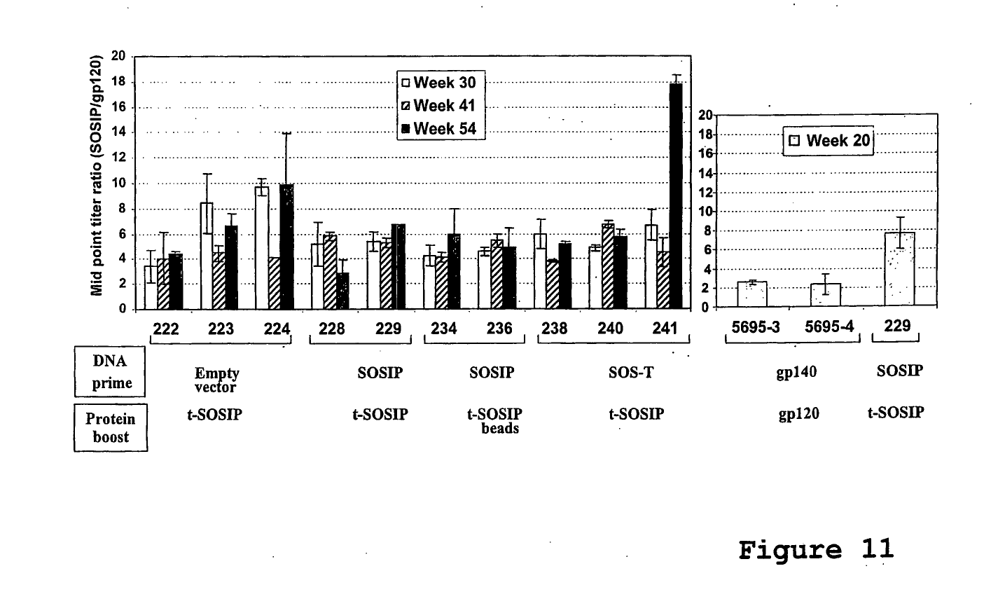

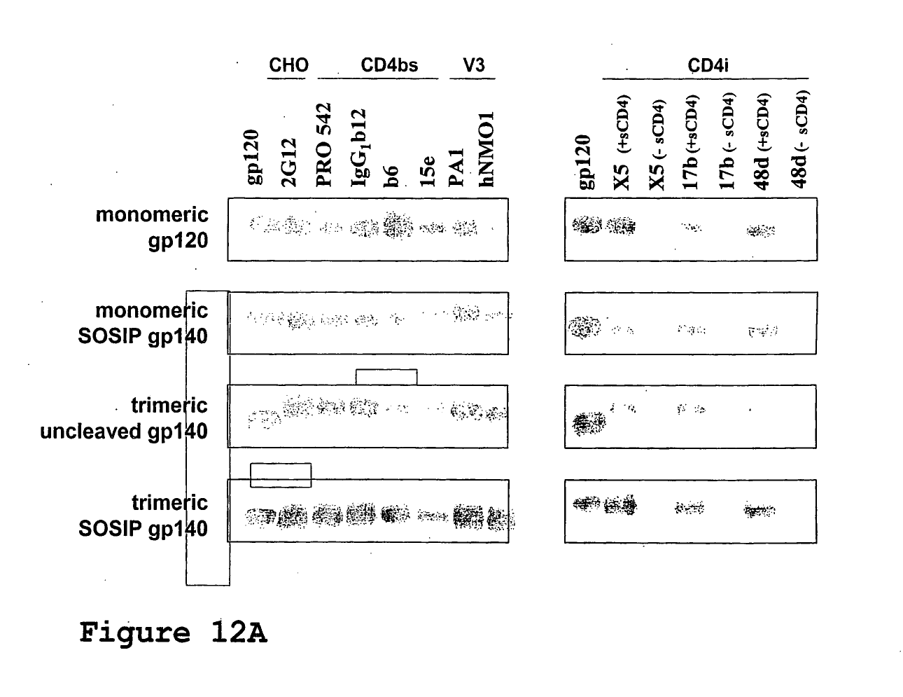

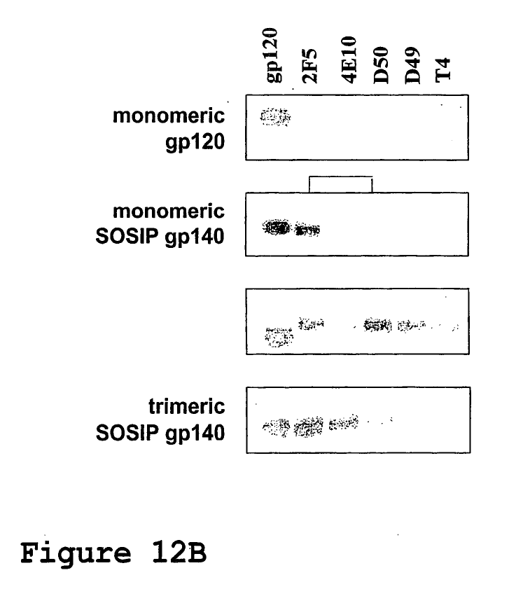

Antibodies in sera specific to gp120 and trimeric SOSIP were quantified by an ELISA assay (Binley et al., 1997a; see FIGS. 8-10). Briefly, purified trimeric SOSIP, monomeric SOSIP, or monomeric gp120 were captured to microtiter plates via lentil lectin (Sigma; St. Louis, Mo.) and incubated with serial dilutions of serum samples from individual rabbits in triplicate wells. Antibodies bound to SOSIP were detected calorimetrically using alkaline phosphatase-labeled anti-rabbit IgG and the substrate PNPP (p-nitrophenyl phosphate; Pierce; Rockford, Ill.). Mid-point titers (50% maximal) were calculated for each group as defined by the antibody dilution giving half-maximal binding after background subtraction (wells with no antigen). The results are presented for selected animals as SOSIP:gp120 midpoint titer ratios in FIG. 11. A SOSIP:gp120 ratio of greater than 2 identifies a preferential binding of the serum antibodies to the trimeric SOSIP protein.

### HIV-1 Neutralization Assays

Full-length gp160 proteins were derived from the HIV-1 isolates JR-FL, ADA, YU2 (all obtained from Dr. T. Dragic, Albert Einstein College of Medicine, New York), and SF162 (Dr C. Cheng-Mayer, Aaron Diamond AIDS Research Center, New York). Amphotropic murine leukemia virus Env (MuLV) was obtained from the AIDS Research and Reference Reagent Program (Division of AIDS, NIAID, NIH; contributed by Dr N. Landau). Full length gp160_{MN} was made by PCR amplification of proviral DNA extracted from peripheral blood mononuclear cells (PBMC) acutely infected with HIV-1MN, using a virus stock supplied by Dr. D. Montefiori, Duke University Medical School, NC 27710. The procedure was similar to that described previously (Connor et al., 1996).

The generation of Env-pseudotyped virus stocks in 293T cells by calcium phosphate transfection, and the use of the engineered HIV-1 co-receptor bearing cell lines U87.CD4.CCR5 and U87.CD4.CXCR4 (3000 cells per well) for Env-pseudotyped virus infection, have been described previously (Gordon et al., 1999; Herrera et al., 2003). The amount of input pseudovirus was normalized by infectivity (virus titer) rather than by p24 antigen content. Virus infection was measured by determining luciferase expression in relative light units (RLU). Various components of animal sera can potentially interfere with neutralization assays, particularly those using HIV-1 Env-pseudotyped viruses, by causing non-specific inhibition or enhancement of virus infection and/or luciferase expression. It has been observed that the magnitude of such interference can be both (pseudo)virus-dependent and animal-dependent. To correct for these interfering factors, a pre-immune serum sample from the same animal was processed identically to the post-immune samples in each experiment in order to determine the percentage neutralization at each dilution. Percentage neutralization was defined as [(1−(RLUₚₒₛₜ₋ᵢₘₘᵤₙₑ/RLUₚᵣₑ₋ᵢₘₘᵤₙₑ))×100%]. The effect of this adjustment was, in most cases, negligible; neutralization titers derived using the pre-immune serum correction were usually very similar to those obtained using the standard control wells, containing only pseudovirus and cells, as a reference. Pseudoviruses expressing MuLV Env were also used to detect any non-specific interference, since anti-HIV-1 Env antibodies would not be expected to cross-neutralize them.

The neutralization properties of selected pre- and post-immune sera were also evaluated under contract, by ViroLogics, Inc. (South San Francisco, Calif.), using their automated Phenosense™ HIV Entry neutralization assay (Binley et al., 2004). This method also involves measuring the infectivity of Env-complemented, luciferase-encoding pseudoviruses in a single-cycle assay using CCR5/CXCR4-expressing U87.CD4 cells.

Neutralization assays were also performed using replication-competent HIV-1_{JR-FL} and HIV-1_{MN}. These studies examined virus replication on mitogen-activated PBMC, using p24 antigen production as the readout. They were carried out essentially as previously described (Herrera et al., 2003), except that a washout procedure was performed on day 1 to remove any potentially interfering components of the animal sera. Again, pre- and post-immune sera from each rabbit were tested in the same neutralization assay, to permit the identification of any non-specific interference. SIVmac239 was also included to provide an indication of any non-specific effects of the rabbit sera against replication of a virus that should not be sensitive to any NAb present.

Table 2 shows 50%, 70% and 90% neutralization titers of the individual rabbit sera at week 20 against JR-FL and MN pseudoviruses. Table 5 shows 50% (A), 70% (B), and 90% (C) neutralization titers of the selected rabbit sera at week 39 against CXCR4-using (MN, 3.2P, HVBc2) and CCR5-using (ASDA, YU2, JR-FL, SF162) pseudoviruses. MLV served as a negative control to identify nonspecific effects.

### Immunoglobulin (Ig) Purification

Total Ig was purified from final bleed rabbit sera using the T-Gel Purification Kit (Pierce, Inc., Rockford, Ill.) according to the manufacturer's instructions, except that an azide-free buffer (50 mM NaH₂PO4, pH 8) was used for antibody elution. The amount of recovered rabbit IgG was quantified using the Easy-Titer Rabbit IgG Assay Kit (Pierce, Inc.), the average recovery being 70% (n=10). The extent of IgG recovery for each individual serum sample was always taken into account when determining neutralization or ELISA titers, to allow comparisons between purified IgG and the corresponding unfractionated serum.

Antibody Depletion from Sera by gp120 or V3-Loop Peptides

A cyclic V3_{JR-FL} peptide (Sequence: Ac-CTRPNNNTRKSIHIGPGRAFYTTGEIIGDIRQAHC-NH₂) (SEQ ID NO: 14), gp120_{JR-FL}, or bovine serum albumin (BSA; Sigma) were coupled to cyanogen bromide (CNBr)-activated Sepharose 4B beads according to the manufacturer's instructions (Amersham Biosciences). Briefly, the beads (0.5 g of powder) were hydrated and washed extensively in 1 mM HCl, washed again in ˜1.75 ml of coupling buffer (0.1 M NaHCO₃, 0.5 M NaCl, pH 8.3) and then resuspended in the coupling buffer to form a ˜50% slurry. The slurry was divided into three equal portions and incubated overnight at 4° C. with either BSA (1 mg), the cyclic V3_{JR-FL} peptide (1 mg), or gp120JR-FL (0.5 mg). After the incubation, the beads were washed extensively in coupling buffer, and excess binding sites were blocked in 0.1 M Tris-HCl (pH 8.0) for 2-3 h at room temperature. Any non-covalently associated proteins were removed by washing first in 0.1 M sodium acetate, 0.5 M NaCl, pH 4.0 and then in Tris-salt buffer (0.1 M Tris-HCl, 0.5 M NaCl, pH 8.0). Serum samples (100 μl) were diluted in Tris-salt buffer then added to sterile 1.5-ml Eppendorf tubes containing ˜50 μl of 50% slurry for overnight incubation at 4° C. The treated (bead-depleted) serum was recovered by low-speed centrifugation (3000×g for 3 min).

The beads were washed extensively in Tris-salt buffer and bound antibodies were subsequently eluted in 0.2 M glycine (pH 2.5-3.0). The pH of the eluted solution was immediately neutralized using 1 M Tris buffer (pH 8.5). The eluted antibodies were concentrated using sterile microcentrifuge-sized filter units (Millipore) and subsequently resuspended in a volume of PBS corresponding to the starting volume of the original serum. All buffers had been passed through 0.2 μm filters prior to use, to help preserve the sterility of the antibody preparations.

Neutralization experiments with the bead-depleted sera or the eluates form the beads were performed as described above.

### Results

Expression and Characterization of Trimeric SOSIP.R6 gp140 Protein

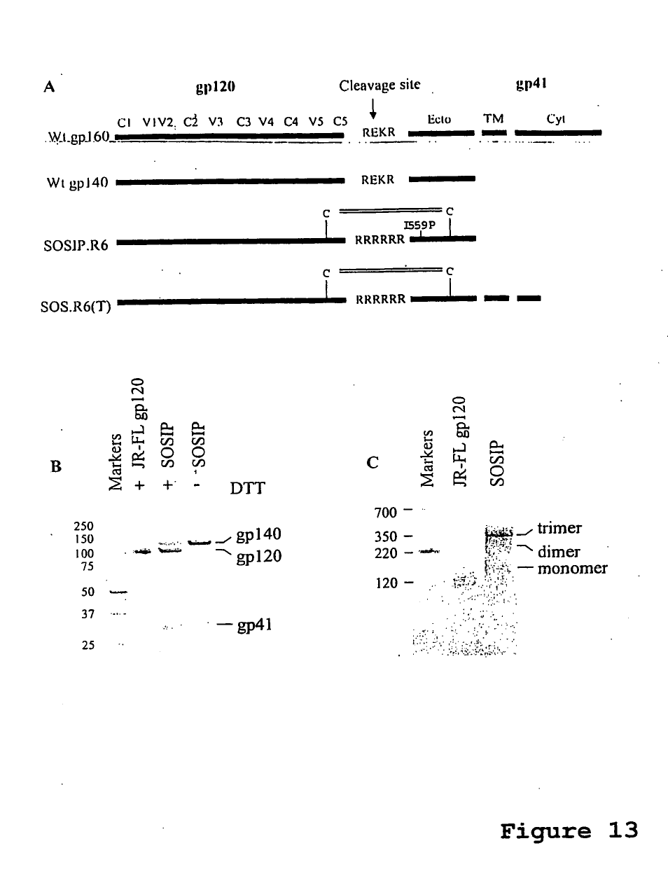

The designs of the Env proteins encoded within the various plasmids used for DNA and protein immunization purposes are summarized in FIG. 13A. When 293T cells were transiently transfected in vitro, it was found that ˜5-fold less of the codon-optimized JR-FL env gene was needed to express the same amount of Env protein than when the natural gene was used. Hence the codon-optimized gene was used to produce preparative amounts of protein for immunizations. The purified SOSIP.R6 gp140 protein was approximately 90% proteolytically processed, such that only a minor fraction (˜10%) of the 140 kDa band remained after treatment with the reducing agent dithiothreitol (DTT) (FIG. 13B, Lane 3). No detectable amounts of free gp120 were present in the SOSIP.R6 gp140 preparation, indicating that the inter-subunit disulfide bond remained substantially intact during purification. Although the amount of disulfide-linked aggregates varied among production lots, the overall amount was consistently ≦20% (FIG. 13B, Lane 4). The major protein contaminants seen on a reducing SDS-PAGE gel migrated as bands with molecular weights of ˜170 kDa and ˜25 kDa. The ˜170 kDa band was identified as bovine alpha 2-macroglobulin by its reactivity with specific antibodies in a western blot assay. The identity of the ˜25 kDa band is not known.

Considering the amount of uncleaved protein, the presence of impurities and aggregates, the overall purity of the trimeric SOSIP.R6 gp140 protein purified from the supernatants of transiently transfected 293T cells was −75%. The trimeric nature of the purified protein was confirmed by Blue-Native PAGE analysis (FIG. 13C) and by analytical size exclusion chromatography.

Immunogenicity of Monomeric gp120_{JR-FL}—Pilot Study

As a prelude to studies with SOSIP.R6 gp140, a Pilot experiment was first conducted using a DNA prime, subunit protein boost format (FIG. 14A), to determine, in particular, the optimal concentration of the boosting antigen to use in future studies. In this first study, a soluble wild-type gp140-expressing construct (gp140 Wt) was used in the DNA-priming phase, with monomeric gp120_{JR-FL} as the boosting antigen. Each animal (two per experimental group) was primed with DNA at weeks 0 and 4, then received two boosts of QS21-adjuvanted gp120 (10 μg, 30 μg or 100 μg) at weeks 12 and 16. A fourth group of animals was primed with a plasmid that lacked an env insert (“empty vector” control), before receiving two gp120 immunizations (30 μg) at weeks 12 and 16. All eight animals were bled every four weeks for 20 weeks.

The outcome of the immunizations was gauged initially by determining anti-gp120 binding antibody midpoint titers by ELISA (FIG. 14B). Endpoint titers, defined as the maximum dilution that yielded a signal two-fold above background, were typically greater than the midpoint titers but were considered to be less useful as comparators. It was found that DNA priming made a substantial difference to the speed and magnitude of the antibody responses to the subsequent protein boosts. Thus, at week 20 (final bleed), the anti-gp120 midpoint titers in the two animals that received only the empty vector control plasmid were 12-fold lower (mean titer, 7×10³), and developed more slowly, than in the animals that had been primed with the DNA plasmid expressing the gp140 Wt protein (mean titer, 9×10⁴). In the latter animals, there was only a weak anti-gp120 response after the DNA-priming phase, but a substantial increase occurred after the first boost with soluble gp120 protein. The second boost had only a minor effect. The amount of gp120 given as the protein boost (10-100 μg) made little difference (+3-fold) to the magnitude of the anti-gp120 response. Hence 30 μg was selected as the amount to use in follow-up studies.

### Immunogenicity of SOSIP.R6 Env Trimers—Second-Stage Study

Based on the results of the Pilot study, a SOSIP study was performed to evaluate the immunogenicity of two variants of the SOSIP protein (soluble and membrane-bound) administered in a heterologous DNA prime-protein boost regimen either as purified protein or coupled to superparamagnetic microbeads. A similar format as in the Pilot study was used: DNA priming at weeks 0 and 4, protein boosts at weeks 12 and 16, with 30 pg of protein used in the boosting immunizations (FIG. 15A). The DNA prime consisted of plasmid pPPI4 containing a DNA insert encoding cleavage-enhanced SOSIP gp140(SOSIP.R6) or pPPI4 containing a DNA insert encoding a membrane-bound version of SOSIP gp140(SOSIP.R6(T)). This membrane-bound protein contains the transmembrane domain of gp41 plus three additional amino acids and ends in the sequence V₆₆₆NRV₆₆₉ (numbering based on the sequence of HIV-1_{HXB2} isolate). Plasmid pPPI4 without any insert was also used as a negative control. Two DNA primes were administered on weeks 0 and 4 using in vivo electroporation. As in the Pilot study, blood was drawn from the rabbits every 4 weeks for analysis.

The principal aims of the experiment were 4-fold: first, to obtain information on the absolute immunogenicity of SOSIP.R6 gp140 trimers; second, to compare the immunogenicity of these trimers with gp120 monomers, using sera retained from the Pilot study for this comparison; third, to determine whether bead-immobilized SOSIP.R6 trimers were superior to soluble forms of the same protein at eliciting an antibody response in the boosting phase (compare arms B and D); fourth, to evaluate the relative merits of soluble (SOSIP.R6 gp140) or membrane-associated (SOS.R6(T)) forms of Env proteins in the DNA priming phase (compare arms B and E). In one arm (arm A), DNA priming was with the control “empty vector” only; in another (arm C), beads lacking Env proteins were used as a control in the boosting phase.

Anti-gp120 binding antibody midpoint titers were again determined, to provide an initial measure of the overall immunogenicity of the test antigens (FIG. 15B). The limitations of DNA immunizations without the later use of an Env-based boosting antigen were clearly revealed by the outcome of arm C, in which the anti-gp120 titers never exceeded 1×10³. Conversely, although an anti-gp120 response did develop when non-primed animals were immunized with SOSIP.R6 gp140 trimers, the titers were much lower than seen in the DNA-primed animals, at least initially. However, after the second protein immunization, anti-gp120 titers were very similar in all the animals receiving SOSIP.R6 gp140 trimers, whether or not they had been primed earlier with DNA. Thus, at this time, the mean anti-gp120 titers in arms A and B were 2.8×10⁴ and 3.2×10⁴, respectively (FIG. 15B). The anti-gp120 responses induced by priming with the membrane-bound Env (SOS.R6; arm E), or by boosting with the bead-based, particulate form of SOSIP.R6 gp140 trimers (arm D), were, on average, slightly (˜2.5-fold) lower than those in animals primed with SOSIP.R6 and boosted with SOSIP.R6 gp140 trimers (arm B). Thus, at week 20, the mean titers in arms B, D and E were 3.2×10⁴, 1.3×10⁴ and 1.5×10⁴, respectively.

When the anti-gp120 titers induced by Env trimers were compared with those raised in response to monomeric gp120 in the Pilot study, the trimers were found to be markedly less immunogenic. Thus, the SOSIP.R6 gp140 trimers elicited anti-gp120 titers typically ˜3-fold lower than had been induced by monomeric gp120, i.e., ˜3×10⁴ compared to ˜1×10⁵ (FIG. 15B).

Since it was considered possible that additional immunizations might increase the quality or quantity of the antibodies raised to the Env trimers, the Second-stage study was extended for another 34 weeks by further boosting the animals. Thus, the rabbits were re-immunized with the same forms of Env protein, or with control beads, at weeks 20, 28, 37 and 52 (FIG. 15A). This extension to the study meant that no gp120 comparison arm was available for time-points beyond week 20, but it was felt that the additional information that might be obtained could still be useful to the design of future experiments.

The additional immunization at week 20 did not further boost the anti-gp120 titers in any of the rabbits from any arm, suggesting that the response to the first two boosts (weeks 12 and 16) was already maximal. This supposition was reinforced by the outcome of the protein boost at week 28. By that time, the anti-gp120 titers in the DNA-primed animals had decayed slightly (3-4-fold) from the week 20 level, whereas the mean titers in animals that had not received a DNA prime (arm A) had decayed by over 30-fold. After the week 28 boost, the anti-gp120 titers rose to levels very similar to those achieved after the week 16 boosting. The further boosts at weeks 37 and 52 were also unable to further increase the maximum anti-gp120 titers; if anything, the responses to the later boosts were a little less than to the boosts at weeks 16 and 28 (FIG. 15B). Although the anti-gp120 titers in arms A and B were similar at week 20, the average titers were ˜3-fold higher at weeks 24 and 28 in the animals that had received the SOSIP.R6 DNA prime (arm B) than in the corresponding non-DNA primed animals (arm A) (p<0.05; Mann Whitney U test). Thus, it is possible that DNA priming may have induced a slightly more robust immune response.

It was also considered worth evaluating whether switching adjuvants from QS-21 to Enhanzyn™ could increase the strength of the immune response to Env antigens. Enhanzyn™ is a monophosphoryl lipid A (MPL) adjuvant which, in addition to MPL, comprises mycobacterial cell wall skeleton and squalene. Hence, from week 37 onwards, the SOS.R6(T)-primed and SOSIP.R6 gp140-immunized animals (arm E) were boosted with Env proteins formulated in Enhanzyn™ adjuvant. Prior to week 37, the gp120 binding titers for animals in arm B (SOSIP.R6 prime with SOSIP.R6 gp140 trimer boost) and arm E were comparable. This modification to the procedure did not, however, have any effect on the ensuing anti-gp120 titers; between weeks 39 and 54, the anti-gp120 titers were not significantly different for animals in arms E and B (Wilcoxon Paired Signed Rank test, p>0.05).

Immunogenicity of Trimeric gp140 Protein Affixed to Microbeads

The use of superparamagnetic microbeads to potentiate immune responses to trimeric SOSIP gp140 antigens of interest, affixed to the microbeads with an anti-gp120-specific antibody, 2G12, was also tested. It has previously been demonstrated that immune responses using these microbeads in mice were approximately one order of magnitude greater than in animals receiving gp120 immunogen without beads (PCT

International Application Publication No. WO 03/022869 A2). Thus, superparamagnetic microbeads may augment the immune response to captured antigen, and this technology may have utility for vaccine development. The use of superparamagnetic beads was combined with the prime-boost regimen in rabbits (FIG. 5).

The DNA primes using pPPI4 or pPPI4-SOSIP.R6(T) were followed by protein boosts with purified trimeric SOSIP gp140. The DNA primes using pPPI4-SOSIP.R6 were followed by protein boosts with purified either trimeric SOSIP gp140, trimeric SOSIP gp140 attached to μMACS Protein G microbeads via 2G12 MAb, or μMACS Protein G-2G12 microbeads only. The boosts were administered by intramuscular injection on weeks on weeks 12, 16, 20, 28, 37, and 52 (see FIG. 7). The saponin QS-21 was used as an adjuvant in all the protein boosts except for Study Arm E (pPPI4-SOSIP.R6(T) as DNA prime and tSOSIP for the protein boost), where Enhanzyn™ was used instead of QS-21 as adjuvant for the immunizations on weeks 37 and 52. The injected rabbits were bled on weeks 0, 4, 8, 12, 20, 24, 28, 30, 37, 39, 41, 52, and 54 (see FIG. 7) and sera were separated for analysis of the anti-SOSIP humoral response by ELISA and for neutralizing activity in well established HIV-1 infectivity assays (Trkola, 1998).

The results of the ELISA assays are shown in FIG. 15B. Temporal analysis of sera demonstrated that immune responses increased after each immunization with DNA primes and peaked by week 16 after the second protein boost. As shown in FIG. 11, rabbits immunized with the SOSIP constructs generated antibodies that preferentially react with the trimeric Env.

### Neutralization of HIV-1 Env-Pseudotyped Viruses

Since anti-gp120 binding antibodies are not a measure of functional anti-Env responses, the neutralization titers at several time-points in both the Pilot and Second-stage studies were measured using HIV-1_{JR-FL} and HIV-1_{MN} Env-pseudotyped viruses in a reporter gene assay (Table 2). The former virus represents the autologous strain for Env-based immunogens and is a primary isolate of average neutralization resistance. HIV-1_{MN} was used as a second pseudovirus in the initial round of neutralization studies. HIV-1_{MN} is a T-cell line-adapted (TCLA) virus that has been used extensively in vaccine-related research over the past decade (Garber et al., 2004) and is sensitive to neutralization by virtue of its in vitro passage history. The use of the corresponding HIV-1_{MN} pseudovirus therefore allowed the detection and quantification of low levels of NAb, although such antibodies may have little relevance to the neutralization of more resistant primary viruses (Parren et al., 1997b). Each serum was tested at least twice.

When the week 20 sera were tested against the more sensitive HIV-1_{MN} pseudovirus, neutralization was detected more frequently, at both the 50% and 90% levels, and the responses could be titrated (Table 1). The strongest and most consistent neutralization of HIV-1_{MN} was seen with sera from arms B (SOSIP.R6 gp140 prime and boost) and A (no prime, SOSIP.R6 gp140 boost). Boosting with bead-captured SOSIP.R6 gp140 was inferior to boosting with the same protein free in solution (compare arms D and B). However, the response to HIV-1_{MN} in one bead-boosted animal, #236, was the strongest observed in the entire study. A comparison between the use of membrane-bound (arm E) and soluble (arm B) forms of Env in the DNA-priming phase suggested that expression of soluble proteins in vivo was the more effective strategy; thus only 3 of the 4 animals in arm E generated any NAbs against HIV-1_{MN}, compared to 4 of 4 in arm B, and at lower titers. The animals receiving two DNA immunizations only (arm C) did not develop any NAb to HIV-1_{MN}, implying that a protein boost in one form or another was necessary for NAb generation. Of note, however, is that the NAb responses to HIV-1_{MN} in the animals receiving SOSIP.R6 gp140 boosts (arms A and B) were very similar to those elicited by monomeric gp120 boosting in the Pilot study. This was particularly so in the two animals given 30 pg of gp120 (5695-3 and 5695-4), the same amount of Env protein used in the Second-stage study. Thus, at 20 weeks, the NAb responses to SOSIP.R6 gp140 trimers and monomeric gp120 were comparable, at least in their ability to neutralize HIV-1_{MN} (Table 1).

None of the week 20 sera raised to any of the gp120 or trimeric gp140 proteins could neutralize HIV-1JR-FL by >70% in the pseudovirus assay, and neutralization of this virus even at the 50% level was rarely observed (Table 1). Thus, sera from only 3/16 SOSIP.R6 gp140 immunized animals (arms A, B, D and E) were positive at the 50% neutralization level, compared with 0/6 sera from animals primed with gp140 Wt DNA and boosted with gp120.

Overall, the NAb responses in the SOSIP.R6 gp140 and gp120 recipients were very similar after 20 weeks. The comparability of the NAb responses to the trimeric and monomeric proteins, despite the markedly lower titers of anti-gp120 binding antibodies in the trimer recipients, suggests that the use of trimeric SOSIP.R6 gp140 Env proteins may reduce the development of non-neutralizing Abs without penalizing NAb induction.

The Second-stage study was continued on an ad hoc basis after week 20, by performing a further 4 immunizations. As noted above, the extension of the study beyond the originally planned 20 weeks eliminates any direct comparison between the responses to the trimeric SOSIP.R6 proteins and those elicited by gp120 monomers in the Pilot study, which ended at week 20. A cross-sectional analysis at week 39 revealed that sera from 9/16 animals (arms A, B, D and E) were now able to neutralize HIV-1_{JR-FL} pseudoviruses by at least 50%, and the titers were increased compared to those observed at week 20 (Table 1). By week 39, animals within the SOS.R6(T) primed group (#238-241) had developed the highest and most consistent response against HIV-1_{JR-FL} among the groups of animals boosted with the SOSIP.R6 gp140 trimer. This difference was not apparent at week 20, when either HIV-1JR-FL or HIV-1_{MN} neutralization titers were inspected. Indeed, serum from one rabbit (#241) had 50% and 70% neutralization titers to HIV-1JR-FL of >160 and 85, respectively, by week 39. Neutralization of HIV-1_{MN} by week 39 sera was generally stronger than that seen at week 20 in the Second-stage study, although when the response was already high at week 20, it increased little if any further by week 39. Thus, the NAb response to the TCLA clone were maximal after only two protein immunizations, while NAb against the autologous primary isolate clone increased with time.

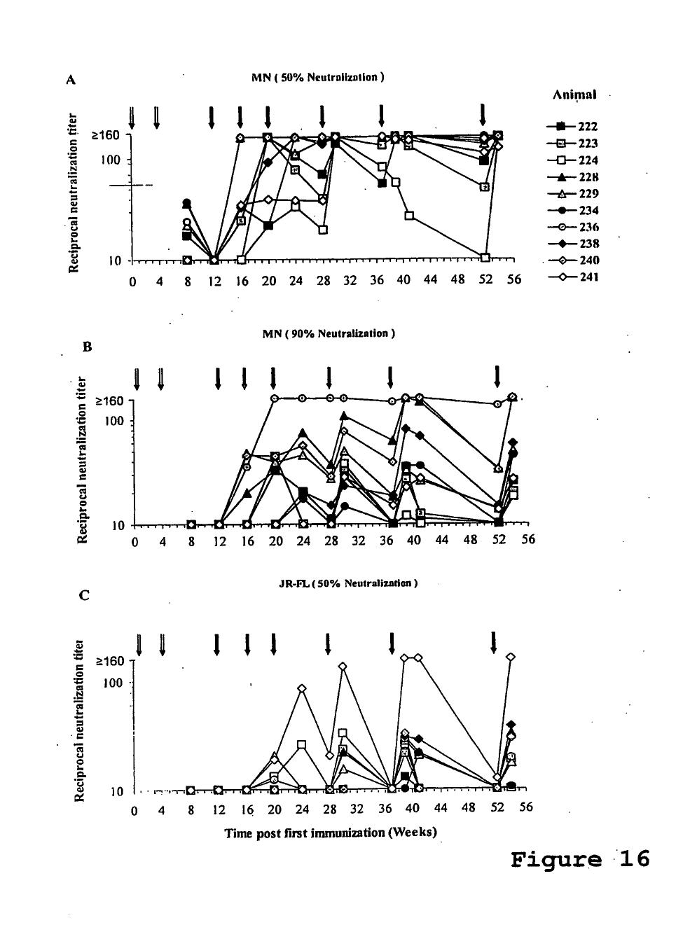

To examine the NAb responses to HIV-1_{MN} and HIV-1_{JR-FL} pseudoviruses in more detail, a panel of sera from ten rabbits in the Second-stage study were selected for further analysis, based on their neutralization potencies at week 39. The animals chosen were #222, #223, #224, #228, #229, #234, #236, #238, #240 and #241 (FIG. 16). A longitudinal analysis allowed us to examine separately the various influences of time, of the use of Enhanzyn adjuvant from week 37 in the SOS.R6(T) primed/SOSIP.R6 gp140-immunized group, and of the additional immunizations themselves.

In 5 of the 10 animals, low levels of NAb against HIV-1MN could be detected at week 8, following the two DNA immunizations, although only at the 50% neutralization level (FIG. 16A). Antibodies capable of neutralizing HIV-1_{MN} by 90% were detected from week 16, initially in only 4 of the 10 animals, then later in all animals. The average peak titer was 86, with a range from 28 to >160 (FIG. 16B). The magnitude of the NAb response to HIV-1_{MN} generally followed the immunization schedule. During the 8-15 week periods between immunizations, the NAb titers in some sera decreased by as much as 5-fold, but were then restored, or in some cases increased, by the subsequent protein immunizations. There was also some variation between the animals in this sub-study in the time taken to generate the peak 90% neutralization titer against HIV-1_{MN}. The peak titers were observed at weeks 20 (#223 and #236), 30 (#224, #229 and #241), 39 (#222, #228, #238 and #240) and 54 (#234), and there was no obvious pattern related to the use of a DNA- or a protein-based immunogen.

In contrast to what was seen with HIV-1_{MN}, NAb against HIV-1_{JR-FL} developed more slowly, and at lower titers (FIG. 16C). Antibodies capable of neutralizing HIV-1_{JR-FL} by 50% were detectable only after the two DNA and two protein immunizations, from week 20 onwards. Although all 10 selected animals did generate NAb against HIV-1JR-FL over this extended study period (weeks 20-54), almost all responses were modest and transient (50% titers <40). Sera from animal #241 (SOS.R6(T) prime, SOSIP.R6 gp140 boost) were capable of neutralizing HIV-1_{JR-FL} by >90% at weeks 41 (titer, 10) and 54 (titer, 24), at which times the 50% titers were >160, but none of the other selected sera could do so. The relatively high titer NAb response of animal #241 was unlikely to be attributable to the use of Enhanzyn™ adjuvant from week 37 onwards, since atypically potent NAb titers were also observed in this animal between weeks 24 and 37. However, it is possible that Enhanzyn™ improved the NAb responses elicited in animals #238 and #240. Sera from both animals lacked neutralizing activity against HIV-1_{JR-FL} prior to the switch to Enhanzyn™ at week 37, but by week 39 50% titers of 34 and 25 had developed (FIG. 16C).

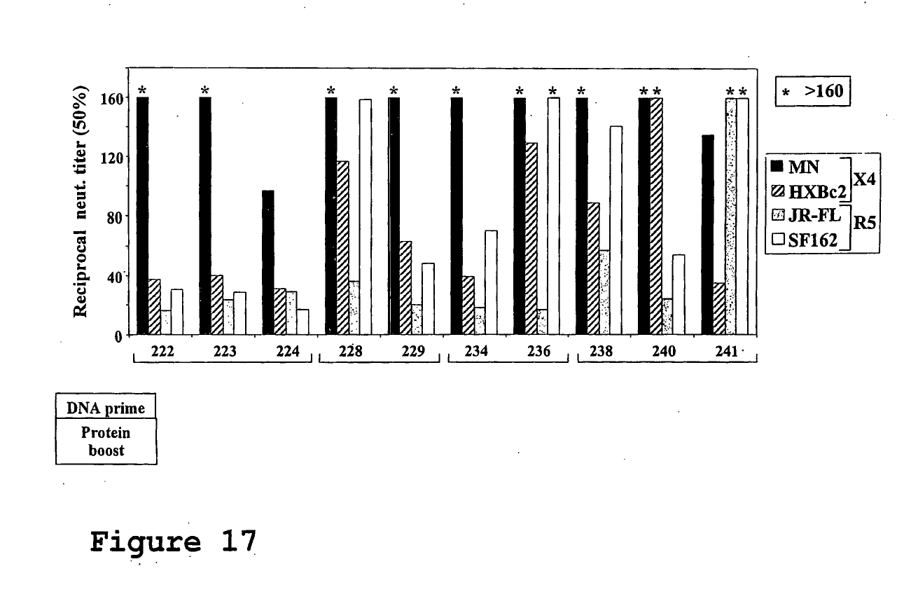

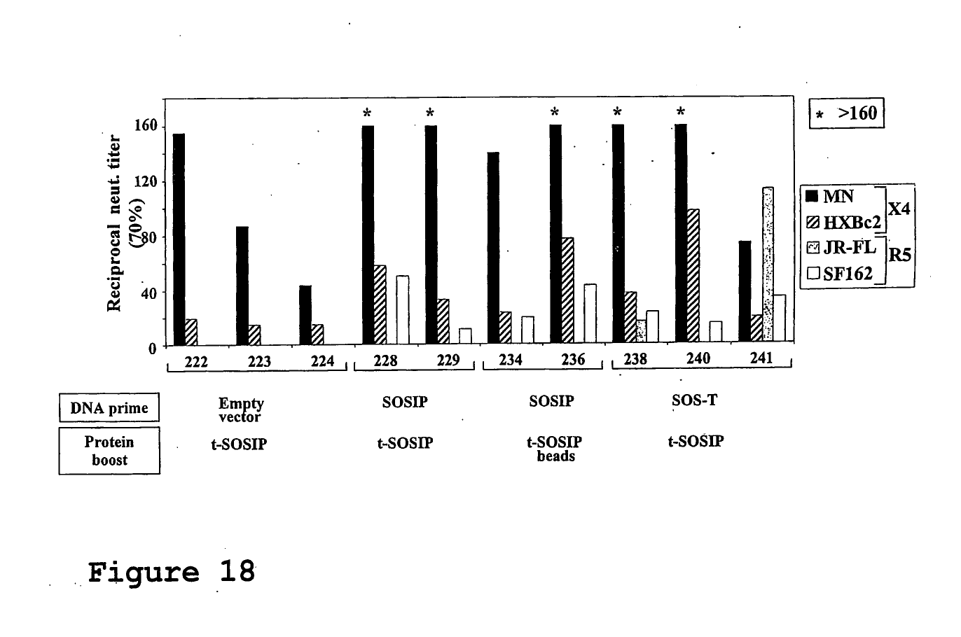

The neutralization data are summarized in Tables 2 and 3, and shown graphically in FIGS. 17 and 18.

TABLE 2
Neutralization of Primary and TCLA Env-pseudotyped HIV-1 by gp120- and SOSIP.R6-immunized Rabbits

| | | | Neutralization of Env-pseudotyped HIV-1 by | | | | | | | | | | | |
|---|---|---|---|---|---|---|---|---|---|---|---|---|---|---|
| | | | rabbit sera from week 20 and week 39 ^{a} | | | | | | | | | | | |
| | | | Week 20 | | | | | | Week 39 | | | | | |
| | | | JR-FL | | | MN | | | JR-FL | | | MN | | |
| Study | DNA/Protein | Animal | 50% | 70% | 90% | 50% | 70% | 90% | 50% | 70% | 90% | 50% | 70% | 90% |
| Pilot | gp140 Wt/ | 5695-1 | — | — | — | 14 | — | — | | | | | | |
| | gp120 | 5695-2 | — | — | — | >160 | >160 | 97 | | | | | | |
| | | 5695-3 | — | — | — | >160 | 124 | 45 | | | | | | |
| | | 5695-4 | — | — | — | >160 | 158 | 66 | | | | | | |
| | | 5695-5 | — | — | — | 102 | 40 | 17 | | | | | | |
| | | 5695-6 | — | — | — | 38 | 25 | 13 | | | | | | |
| Second | Empty vector/ | 222 | — | — | — | >160 | 98 | 33 | — | — | — | 148 | 92 | 35 |
| stage | SOSIP.R6 | 223 | — | — | — | >160 | 141 | 45 | 21 | — | — | >160 | 101 | 27 |
| | Arm A | 224 | 12 | — | — | 22 | 11 | — | 22 | — | — | 57 | 28 | 12 |
| | | 225 | — | — | — | >160 | 103 | 31 | — | — | — | >160 | >160 | 81 |
| | SOSIP.R6/ | 226 | — | — | — | >160 | 120 | 44 | — | — | — | >160 | >160 | 113 |
| | SOSIP.R6 | 227 | — | — | — | 154 | 84 | 25 | — | — | — | >160 | >160 | 50 |
| | Arm B | 228 | — | — | — | >160 | 125 | 33 | 29 | — | — | >160 | >160 | >160 |
| | | 229 | 21 | — | — | >160 | 138 | 39 | 20 | — | — | >160 | 129 | 33 |
| | SOSIP.R6/ | 230 | — | — | — | — | — | — | — | — | — | — | — | — |
| | Empty beads | 231 | — | — | — | — | — | — | — | — | — | 20 | — | — |
| | Arm C | 232 | — | — | — | — | — | — | — | — | — | 16 | — | — |
| | | 233 | — | — | — | — | — | — | — | — | — | — | — | — |
| | SOSIP.R6/ | 234 | — | — | — | 22 | — | — | — | — | — | >160 | 127 | 36 |
| | SOSIP.R6 beads | 235 | — | — | — | 15 | — | — | — | — | — | >160 | 109 | 24 |
| | Arm D | 236 | — | — | — | >160 | >160 | >160 | 21 | — | — | >160 | >160 | >160 |
| | | 237 | — | — | — | — | — | — | — | — | — | >160 | 104 | 22 |
| | SOS.R6(T)/ | 238 | — | — | — | 91 | 28 | — | 34 | 14 | — | >160 | >160 | 81 |
| | SOSIP.R6 | 239 | — | — | — | — | — | — | 22 | — | — | >160 | 153 | 30 |
| | Arm E | 240 | — | — | — | >160 | 111 | 45 | 25 | — | — | >160 | >160 | >160 |
| | | 241 | 16 | — | — | 39 | 19 | — | >160 | 85 | — | 150 | 76 | 22 |

^{a} The reciprocal of the dilution which resulted in 50%, 70% or 90% neutralization of the primary (JR-FL) or TCLA (MN) Env-pseudotyped HIV-1 infectivity on co-receptor bearing U87.CD4 cells (mean of 2-6 tests). A dash indicates <50% neutralization was observed at the 1:10 dilution. Note the Pilot study was terminated after 20 weeks, so no formal comparison of responses at week 39 was possible

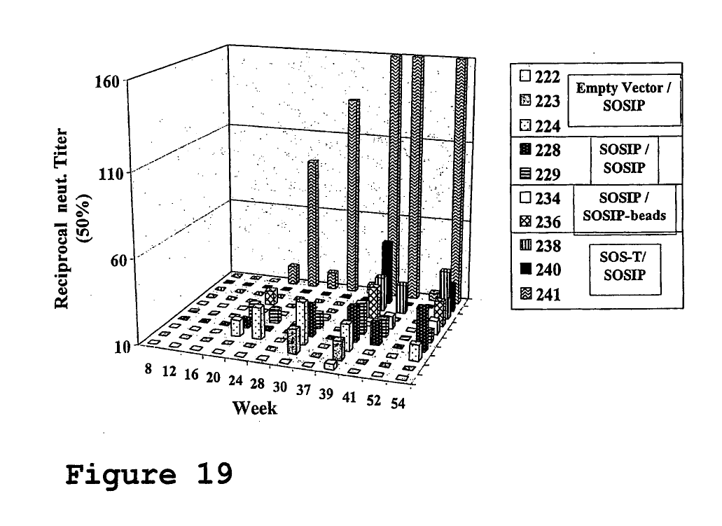

A time course of neutralization titers against HIV-1_{JR-FL} pseudoviruses is shown in FIG. 19. 50% neutralization titers in the sera of different rabbits generally peaked around week 39 and remained approximately constant or declined slightly.

### Neutralization of PBMC-Grown, Replication-Competent Virus

Pseudovirus-based assays are typically more sensitive than those based on the use of PBMC-grown virus with PBMC as the target cells (Moore and Burton, 2004). The latter assay has been the generally accepted standard for many years. Hence, selected final bleed (week 54) sera were compared for their abilities to neutralize both replication-competent HIV-1_{JR-FL} and HIV-1_{MN} in a PBMC assay, and the corresponding Env-pseudotyped viruses on the appropriate U87.CD4 co-receptor bearing cells (Table 3). SIVmac239 and MuLV Env-pseudotyped viruses served as negative controls for the PBMC and pseudovirus assays, respectively. None of the rabbit sera neutralized these control viruses, suggesting that any neutralization of HIV-1JR-FL and HIV-1_{MN} was likely to be a specific effect, and not due to toxicity or other interfering factors.

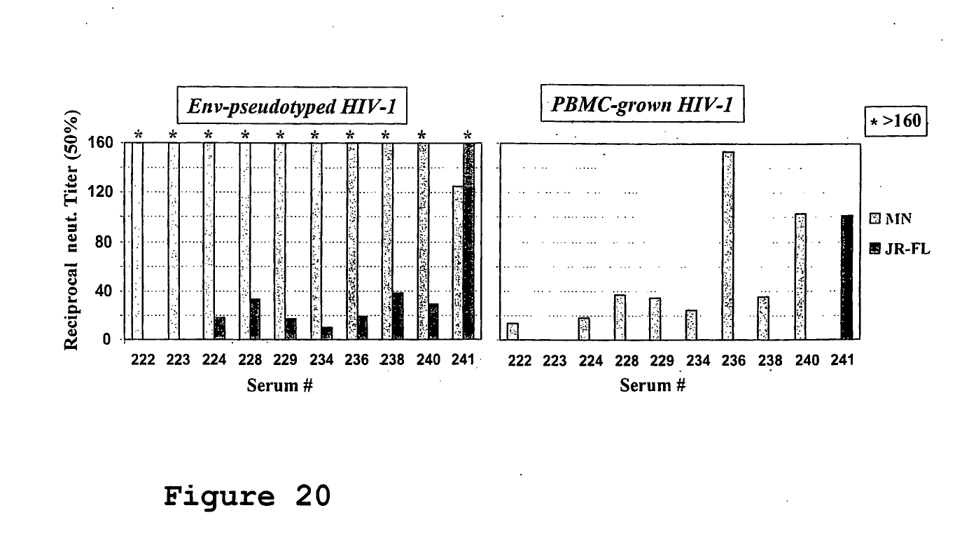

The infectious virus-based PBMC assay and the Env-pseudotyped assay in U87.CD4 cells generated broadly similar patterns of neutralization data, although as expected the PBMC assay was substantially the less sensitive of the two (FIG. 20; Table 3). The greater neutralization sensitivity of the Env-pseudotyped viruses is also shown by the use of CD4-IgG2 as a reference reagent. Thus, HIV-1_{JR-FL} pseudovirus was approximately three times more sensitive to CD4-IgG2 at the 50% level (mean titer, 0.11±0.07 μg/ml; n=29) compared to PBMC-grown HIV-1_{JR-FL} (0.32±0.24 μg/ml; n=14). At the 90% neutralization level, the differential was approximately two-fold (HIV-1_{JR-FL} pseudovirus, 0.72±0.44 μg/ml; replication-competent HIV-1JR-FL, 1.32±0.74 μg/ml). A greater differential (7-10-fold) was seen between

TABLE 3
Comparative neutralization of Env-pseudotyped and PBMC-grown HIV-1

| | Neutralization of Env-pseudotype or PBMC-grown HIV-1 by | | | | | | | | | | | |
|---|---|---|---|---|---|---|---|---|---|---|---|---|
| | sera from SOSIP.R6 gp140-immunized Rabbits ^{a} | | | | | | | | | | | |
| | Env-pseudotyped HIV-1 | | | | | | PBMC-grown HIV-1 | | | | | |
| | JR-FL | | | MN | | | JR-FL | | | MN | | |
| Animal | 50% | 70% | 90% | 50% | 70% | 90% | 50% | 70% | 90% | 50% | 70% | 90% |
| 222 | — | — | — | >160 | 111 | 26 | — | — | — | 14 | — | — |
| 223 | — | — | — | >160 | 74 | 20 | — | — | — | — | — | — |
| 224 | 18 | — | — | >160 | 65 | 18 | — | — | — | 18 | — | — |
| 228 | 33 | 10 | — | >160 | >160 | >160 | — | — | — | 37 | 26 | 14 |
| 229 | 17 | — | — | >160 | >160 | 50 | — | — | — | 34 | 22 | 11 |
| 234 | 10 | — | — | >160 | >160 | 45 | — | — | — | 24 | 12 | — |
| 236 | 19 | — | — | >160 | >160 | >160 | — | — | — | 154 | 100 | 45 |
| 238 | 38 | 15 | — | >160 | >160 | 59 | — | — | — | 35 | 21 | — |
| 240 | 29 | — | — | >160 | >160 | >160 | — | — | — | 103 | 67 | 29 |
| 241 | >160 | >160 | 24 | 125 | 75 | 26 | 101 | 56 | — | — | — | — |

^{a} The reciprocal of the dilution which resulted in 50%, 70% or 90% neutralization of Env-pseudotyped HIV-1 infectivity of co-receptor bearing U87.CD4 cells, or PBMC-grown HIV-1 on PBMC. A dash indicates <50% neutralization was observed at the 1:10 dilution.

HIV-1MN pseudoviruses (50% titer, 0.01±0.01 μg/ml; 90% titer, 0.05±0.06 μg/ml; n=22) and PBMC-grown HIV-1JR-FL (50% titer, 0.10±0.09 μg/ml; 90% titer, 0.38±0.57 μg/ml; n=6).

Only four of the test sera from SOSIP.R6 gp140-immunized animals (#228, #229, #236 and #240) could neutralize HIV-1_{MN} by 90% (mean titer, 25) in the PBMC assay (Table 3). Three of these sera (#228, #236 and #240) were the most potent against HIV-1_{MN} in the pseudovirus assay (90% titers >160). However, sera from only one SOSIP.R6 gp140-immunized animal (#241) neutralized JR-FL by >50% in PBMC, whereas sera from 8/10 animals could neutralize HIV-1JR-FL at the 50% level in the Env-pseudotype assay. When the longitudinal serum set from animal #241 was tested, the rise and fall of the neutralization titers against PBMC-grown HIV-1_{JR-FL} and HIV-1_{JR-FL} Env pseudotype virus followed a similar temporal pattern, although the absolute titers were lower in the PBMC assay (data not shown). Hence the principal difference between the two assay systems is quantitative rather than qualitative.

### Breadth of Antibody Reactivity Elicited by Env Subunit Protein Immunization

While the ability of animal antisera to neutralize the homologous strain on which the subunit immunogen was based is a relevant parameter, their breadth of reactivity against a range of heterologous isolates is likely to be a more meaningful measure of the potential of any candidate HIV-1 immunogen (Burton et al., 2004; Garber et al., 2004).

The cross-neutralization activity of the panel of ten selected final bleed rabbit antisera (see Table 3) was first assessed against an extended virus test panel that included the Env-pseudotype viruses HIV-1_{SF162}, HIV-1_{ADA} and HIV-1_{YU2}, and a panel of 7 PBMC-grown, primary isolates. Selected sera from gp120- and SOSIP.R6 gp140-immunized animals were also tested by an external laboratory, ViroLogics Inc.

The atypically sensitive HIV-1_{SF162} Env-pseudotyped virus was neutralized to high titer by sera from gp120-immunized animals in the Pilot study (mean 50% neutralization titer±SD, 108±4), and by sera from the SOSIP.R6 gp140-immunized animals (titer, 112±43). However, none of these antisera could neutralize the more resistant HIV-1_{ADA} or HIV-1_{YU2} Env-pseudotyped viruses, even at the 50% level, at dilutions of 1 in 10 (data not shown).

Final bleed sera from animals #228, #236 and #241 were tested against a panel of seven PBMC-grown R5 isolates, representing Env subtypes A (n=2), B (n=3) and C (n=2), in a PBMC assay. No activity (50% neutralization titers <10) was observed against these viruses with any of the test sera (data not shown).

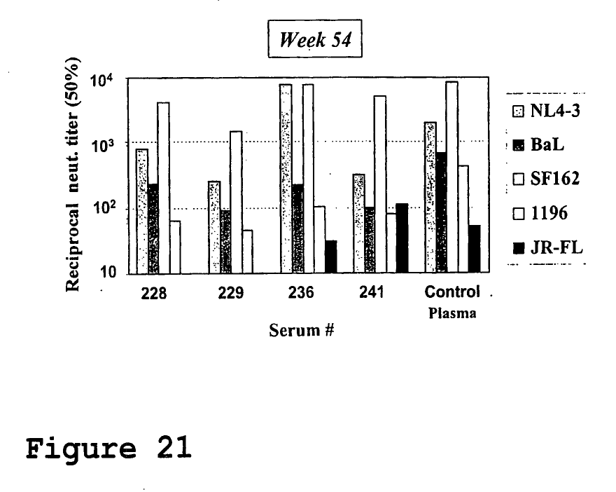

Four sera from the Second-stage study (weeks 20 and 54) and one from the Pilot study (week 20) were also assessed for neutralizing activity using the Phenosense™ HIV Entry Env-pseudotype neutralization assay (FIG. 21). Seven HIV-1 strains were used, including well-characterized env clones with a range of sensitivities to NAb (Table 4). The pseudoviruses used in this assay were recently defined as ranging from highly sensitive to highly resistant as follows: HIV-1_{SF162}, HIV-1_{NL4/3}>HIV-1_{BaL}, HIV-1₁₁₉₆>HIV-1_{JR-FL}, HIV-1_{JR-CSF}>HIV-1₁₁₆₈ (Binley et al., 2004). A similar ranking was observed in the present study. Sera from SOSIP.R6 gp140- or gp120-immunized animals neutralized the 4 most sensitive isolates (HIV-1_{SF162}, HIV-1_{NL/3}, HIV-1_{BaL} and HIV-1₁₁₉₆). Neutralization of the more resistant HIV-1_{JR-FL} virus was, however, only observed using sera from the SOSIP.R6 gp140-immunized animals, #236 and #241. In contrast, the latter two sera were both inactive against HIV-1_{JR-CSF} and HIV-1₁₁₆₈. As was also observed in the in-house

TABLE 4
Assessment of cross-neutralization activity in the Phenosense ™ assay (ViroLogics Inc.)

| | | Neutralization of Env-pseudotyped HIV-1 by Rabbit sera ^{a} | | | | | | | |
|---|---|---|---|---|---|---|---|---|---|
| Animal | Serum (Wk) | SF162 | NL4/3 | BaL | 1196 | JR-FL | JR-CSF | 1168 | MLV |
| 5695-4 | 0 | — | — | — | — | — | — | — | — |
| | 20 | 4658 | 90 | 63 | 32 | — | — | — | — |
| 228 | 0 | — | — | — | — | — | — | — | — |
| | 20 | 2233 | 154 | 55 | — | — | — | — | — |
| | 54 | 4167 | 785 | 230 | 65 | — | — | — | — |
| 229 | 0 | — | — | — | — | — | — | — | — |
| | 20 | 810 | 40 | — | — | — | — | — | — |
| | 54 | 1473 | 247 | 90 | 46 | — | — | — | — |
| 236 | 0 | — | — | — | — | — | — | — | — |
| | 20 | 1499 | 1327 | 29 | — | — | — | — | — |
| | 54 | 7769 | 7895 | 218 | 101 | 32 | — | — | — |
| 241 | 0 | — | — | — | — | — | — | — | — |
| | 20 | 3379 | 36 | 46 | 40 | — | — | — | — |
| | 54 | 5061 | 313 | 100 | 79 | 112 | — | — | — |
| Reference HIV-1 + | Mean | 8497 | 2036 | 678 | 413 | 52 | 133 | 26 | — |
| plasma, N16 | | | | | | | | | |

^{a} The reciprocal of the dilution which resulted in 50% neutralization of Env-pseudotyped HIV-1 infectivity of co-receptor bearing U87.CD4 cells. A dash indicates that <50% neutralization was observed at the 1:25 dilution. Plasma (N16) is from an HIV-1 infected individual and is used as a reference standard to control for assay to assay variation.

pseudovirus assays (Tables 2 and 3), serum from animal #241 was the most potent against HIV-1JR-FL and had modest activity against HIV-1_{MN}, whereas serum #236 had more modest activity against HIV-1_{JR-FL} but strongly inhibited HIV-1_{MN}.

Plasma sample N16, obtained from an HIV-1 subtype B infected individual, was recently selected from a panel of twenty-seven such plasmas for its potent and broadly reactive neutralizing antibody response (Binley et al., 2004). It was used to gauge the neutralizing activity of sera from the gp140 SOSIP.R6-immunized animals, #236 and #241, by performing a comparative analysis. The primary Env-pseudotyped viruses HIV-1 BaL, 1196 and JR-FL were neutralized to a comparable extent by these two rabbit sera and by the N16 plasma (Table 4). Thus, immunization of rabbits #236 and #241 induced NAb of a potency similar to those generated during natural infection, although the breadth of neutralization by the rabbit sera was more restricted.

Overall, there was reasonable concordance between the pseudovirus neutralization data generated using the commercial Phenosense™ assay and the in-house tests. No obviously toxic effects of the test sera were apparent in any of the assays, and the control MuLV Env-pseudotyped virus was not inhibited.

Taken together, the above sets of data demonstrate that immunization with SOSIP.R6 gp140 elicited antibodies capable of neutralizing the relatively resistant, autologous primary isolate HIV-1_{JR-FL} in both pseudovirus and whole-virus formats. A few heterologous Env subtype B Env-pseudotyped viruses that are known to be fairly sensitive to NAbs were also neutralized by some of the test sera. However, the overall ability of the sera to cross-neutralize primary isolates was modest.

### Qualitative Analysis of Neutralizing Antibody Responses

To gain some further understanding of what component was responsible for the neutralizing activity present in these rabbit antisera, a series of control and/or analytical experiments was performed on selected serum samples.

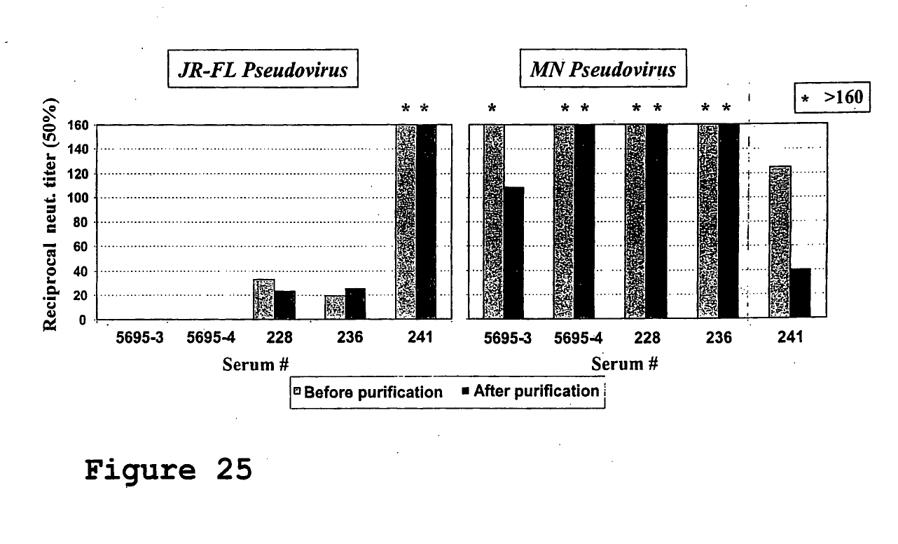

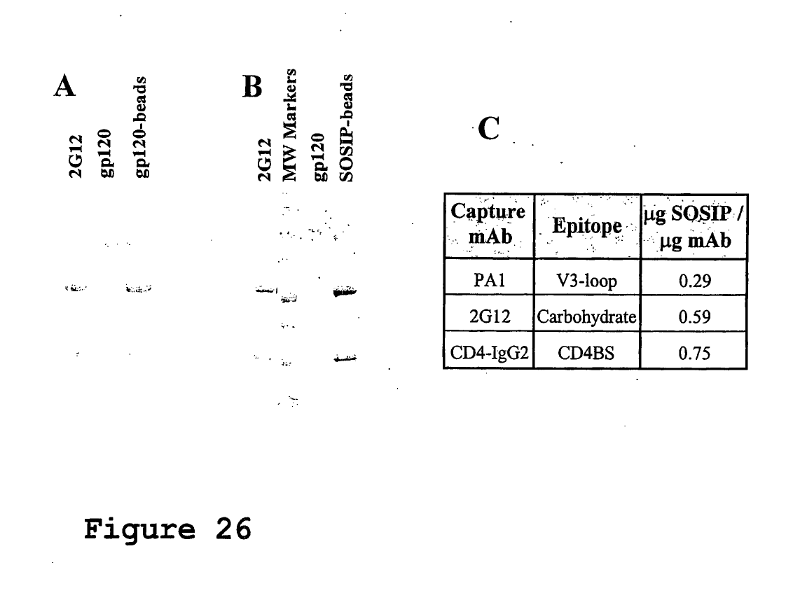

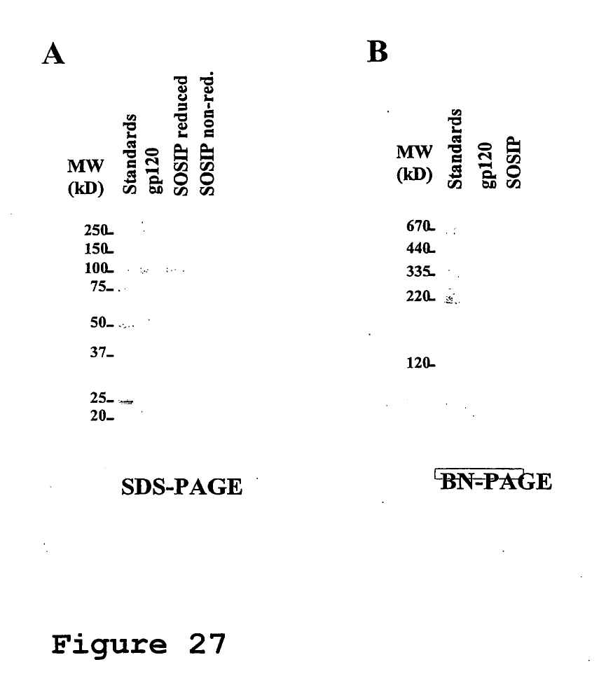

First, Ig fractions from serum samples 5695-2, 5695-3, 5695-4 and 5695-6 from week 20 of the Pilot study, and from sera #228, #236 and #241 from week 54 of the Second-stage study were purified. The abilities of the Ig fractions and the unfractionated sera to neutralize HIV-1JR-FL and HIV-1_{MN} were then compared in our standard, in-house Env-pseudotype assay. The neutralizing activity was retained in the purified Ig fractions, showing that antibodies, and not other antiviral molecules such as chemokines or cytotoxic serum proteins, were responsible for the inhibition of viral infection caused by the corresponding unfractionated sera (FIG. 25). Only minor quantitative differences between the unfractionated sera and the purified Ig fractions were recorded.

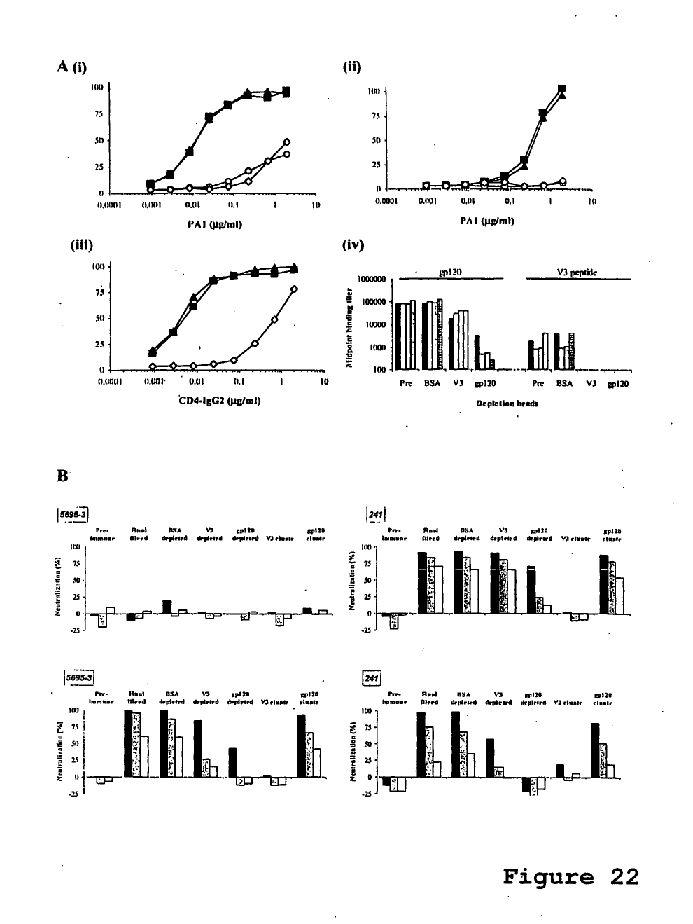

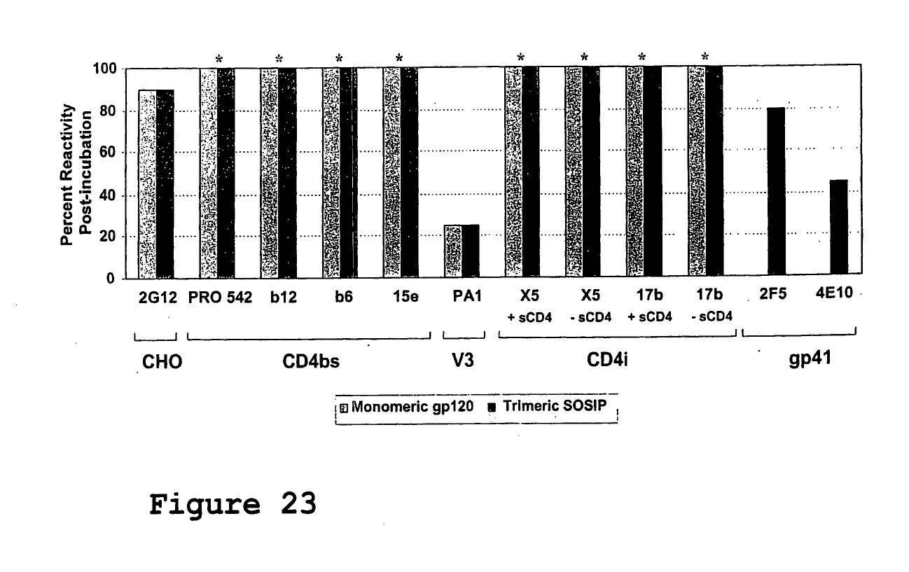

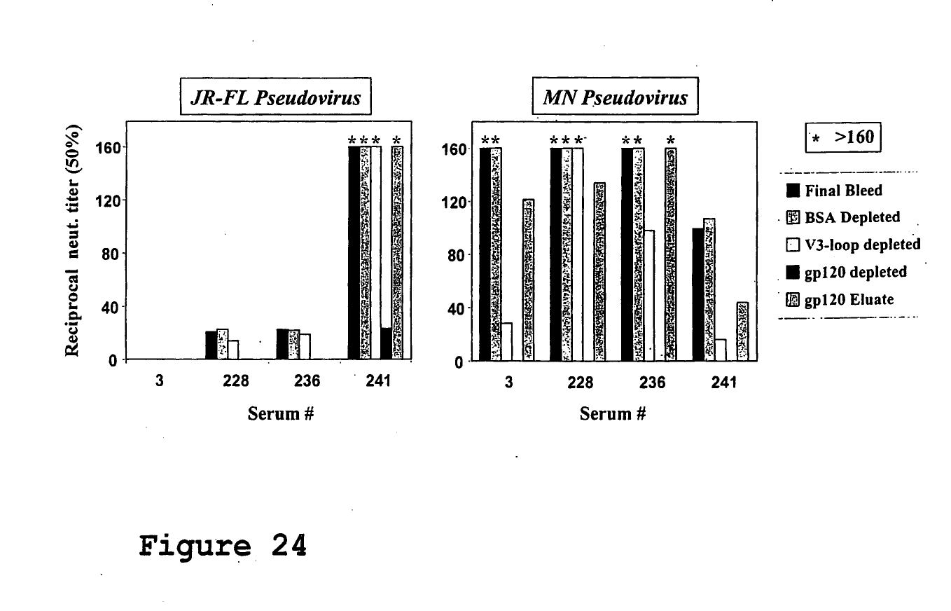

Whether neutralization was attributable to antibodies that recognized JR-FL gp120 and, more specifically, its V3 region was next evaluated. To deplete antibodies directed against gp120 or the V3 loop, selected final bleed sera (those with the highest NAb titers) were incubated with BSA (negative control), gp120_{JR-FL} or a cyclic V3_{JR-FL} peptide affixed to Sepharose 4B beads. The depletion procedure was validated by spiking the anti-V3_{JR-FL} MAb PA1, or the CD4-IgG2 molecule, into a rabbit pre-bleed serum pool, then testing the ability of the gp120- and V3 peptide-containing beads to remove these agents from the spiked serum (FIG. 22A, panels i-iii). Both the gp120- and the V3 peptide-containing beads depleted >99% of the added PA1 MAb from the test serum, and the gp120-beads removed a similar proportion of spiked CD4-IgG2, as judged by the shifts in the titration curves. As expected, the V3 peptide-beads did not remove CD4-IgG2, and neither PA1 nor CD4-IgG2 was removed by the control BSA-beads (FIG. 22A, and data not shown).

When final bleed sera from four selected Env-protein immunized rabbits (Pilot study animal 5695-3, and Second-stage study animals #228, #236 and #241) were treated, the gp120-beads decreased the anti-gp120 titers on average by 33-, 166-, 199- and 423-fold, respectively (FIG. 22A, iv and data not shown). Hence the depletion procedure was effective at removing antigen-induced anti-gp120 Abs from sera. The same four sera were also incubated with the V3 peptide-beads; in this case, the anti-gp120 titer decreased only 4-, 3-, 2- and 3-fold, respectively, implying that only a minor fraction of the total anti-gp120 Abs in the sera could recognize the V3 region of gp120. Both the gp120- and V3 peptide-beads were able to remove essentially all the V3 peptide-reactive Abs from all four animal sera. Similar reductions in gp120 binding titers were observed when three other Pilot study sera, 5695-2, 5695-4 and 5695-6, were depleted using gp120-beads (data not shown).

Rabbit antisera #5695-3 (Pilot study) and #241 (SOS.R6(T) DNA prime, followed by SOSIP.R6 gp140 protein boost) were then tested for their ability to neutralize HIV-1_{JR-FL} and HIV-1_{MN} in the Env-pseudotype assay, before and after bead depletion. Serum #241 was selected because of its relatively strong neutralizing activity compared to most of the other sera; the aim was to determine what Ab specificities were responsible for this activity. Serum sample #5695-3 from the Pilot study was chosen for comparison. The bead-treated and untreated sera, and also pre-immune sera from the same animals, were tested in the neutralization assays at three different dilutions (FIG. 22B).

TABLE 5
Neutralization titers of the individual rabbit sera
at Week 39 for CXCR4- and CCR5-using pseudoviruses

| | CXCR4-using Env Pseudotypes | | | CCR5-using Env Pseudotypes | | | | |
|---|---|---|---|---|---|---|---|---|
| Animal # | MN | 3.2P | HXBc2 | ADA | YU2 | JRFL | SF162 | MLV |

A. 50% Neutralization Titers

| | | | | | | | | |
|---|---|---|---|---|---|---|---|---|
| 222 | >160 | — | 37 | — | — | 16 | 30 | — |
| 223 | >160 | — | 40 | — | — | 23 | 28 | — |
| 224 | 97 | — | 31 | — | — | 29 | 17 | — |
| 228 | >160 | 18 | 117 | — | — | 36 | 159 | — |
| 229 | >160 | — | 63 | — | — | 20 | 48 | — |
| 234 | >160 | — | 39 | — | — | 18 | 70 | — |
| 236 | >160 | — | 129 | — | — | 17 | >160 | — |
| 238 | >160 | — | 89 | — | — | 57 | 141 | — |
| 240 | >160 | 17 | >160 | — | — | 24 | 54 | — |
| 241 | 135 | — | 35 | — | — | >160 | >160 | — |

B. 70% Neutralization Titers

| | | | | | | | | |
|---|---|---|---|---|---|---|---|---|
| 222 | 155 | — | 19 | — | — | — | — | — |
| 223 | 87 | — | 15 | — | — | — | — | — |
| 224 | 44 | — | 15 | — | — | — | — | — |
| 228 | >160 | — | 58 | — | — | — | 50 | — |
| 229 | >160 | — | 33 | — | — | — | 11 | — |
| 234 | 140 | — | 23 | — | — | — | 19 | — |
| 236 | >160 | — | 77 | — | — | — | 43 | — |
| 238 | >160 | — | 37 | — | — | 16 | 23 | — |
| 240 | >160 | — | 98 | — | — | — | 15 | — |
| 241 | 74 | — | 19 | — | — | 114 | 34 | — |

C. 90% Neutralization Titers

| | | | | | | | | |
|---|---|---|---|---|---|---|---|---|
| 222 | 38 | — | — | — | — | — | — | — |
| 223 | 24 | — | — | — | — | — | — | — |
| 224 | 17 | — | — | — | — | — | — | — |
| 228 | >160 | — | 19 | — | — | — | — | — |
| 229 | 42 | — | — | — | — | — | — | — |
| 234 | 38 | — | — | — | — | — | — | — |
| 236 | >160 | — | 26 | — | — | — | — | — |
| 238 | 82 | — | — | — | — | — | — | — |
| 240 | >160 | — | 28 | — | — | — | — | — |
| 241 | 23 | — | — | — | — | 10 | — | — |

As expected, the serum from the gp120-immunized rabbit did not neutralize HIV-1_{JR-FL}, before or after treatment with the gp120- or V3 peptide-beads. Its ability to neutralize HIV-1_{MN} was completely abolished by exposure to the gp120-beads (the dilution providing 50% inhibition [ID_{50%}] decreased from >160 to <10), and it was substantially depleted by the V3 peptide-beads (ID₅₀% 28). The possibility cannot be discounted that some antibodies specific for V3_{MN} may not have been depleted from these sera by use of a V3_{JR-FL} peptide. NAb activity could be recovered from the gp120-beads after glycine elution, but this procedure was unsuccessful at eluting NAb from the V3 peptide-beads, presumably because the affinity of the Ab-peptide interaction was very high. Similar results to those generated with 5695-3 were obtained using final bleed sera from three other gp120-immunized animals (5695-2, 5695-4 and 5695-6), before and after gp120-bead depletion (data not shown).

The HIV-1_{JR-FL} neutralizing activity of antiserum #241 was not measurably affected by treatment with the V3 peptide-beads (the ID₅₀% remained >160), but it was substantially depleted by the gp120-beads (the ID₅₀% decreased to 23). In contrast, the activity of the same serum against HIV-1_{MN} was dependent upon the presence of V3-directed antibodies; their removal by the V3 peptide-beads substantially reduced the titer against HIV-1_{MN} (ID₅₀% decrease from 99 to 16). The gp120-beads had a similar effect (ID₅₀%<10) (FIG. 22B). Similar observations were made using final bleed sera from animals #228 and #236, albeit to a lower titer than #241 (data not shown).

As noted above, the most active rabbit sera could neutralize HIV-1_{JR-FL} pseudovirus (and the neutralization-sensitive HIV-1_{SF162} pseudovirus), but not HIV-1_{ADA} or HIV-1_{YU2} pseudoviruses. The V3 peptide-depletion experiments suggested that the V3 region was unlikely to be the target for these NAbs. However, this issue was explored further by altering the JR-FL V3 sequence to that of ADA by site-directed mutagenesis to create the single-residue change E322D. The resulting HIV-1_{JR-FL}/V3-_{ADA} pseudovirus was then tested to determine whether it was now resistant to neutralization by serum #241. The serum was found to neutralize both HIV-1_{JR-FL} and HIV-1_{JR-FL/V3-ADA} pseudoviruses with identical titers (50% neutralization, >160). Because HIV-1_{ADA} is resistant to serum #241, it seems unlikely that Abs to V3 were responsible for the neutralization of HIV-1_{JR-FL} by this serum. However, the target(s) of the NAbs present in serum #241 remains to be determined. The resistance of HIV-1_{ADA} and HIV-1_{YU2} to neutralization by the same serum is probably due to more global differences in Env configuration, rather than in V3 alone. This conclusion would be consistent with other studies on the global neutralization resistance of various viruses (Moore et al., 1996).

### Discussion

HIV vaccine development targeting HIV envelope glycoproteins has been hindered by the inherent instability of the native envelope glycoprotein complex. It may be possible to overcome this problem by producing more stable forms of the envelope glycoprotein complex that better mimic the native structure.

One approach to resolving the instability of the native complex is to remove the proteolytic cleavage site between the gp120 and gp41 subunits, leading to the expression of a gp140 glycoprotein in which the gp120 subunit is covalently linked by a peptide bond to the gp41 ectodomain (gp41_{ECTO}). Such proteins can be oligomeric, sometimes trimeric (Chen et al., 2000; Earl et al., 1997; Earl et al., 1994; Earl et al., 1990; Earl et al., 2001; Edinger et al., 2000; Farzan et al., 1998; Richardson et al., 1996; Stamatatos et al., 2000; Yang et al., 2000a; Yang et al., 2000b; Yang et al., 2001; Zhang et al., 2001). However, it is doubtful that such uncleaved gp140 (gp140UNC) proteins represent the structure of the native, fusion-competent complex in which the gp120-gp41 cleavage site is fully utilized. Hence, the receptor-binding properties of gp140UNC proteins tend to be impaired, and non-neutralizing antibody epitopes are exposed on them that probably are not accessible on the native structure (Binley et al., 2000a; Burton, 1997; Hoffman et al., 2000; Sattentau et al., 1995; Zhang et al., 2001).

An alternative approach is to retain the cleavage site but to introduce a disulfide bond between the gp120 and gp41_{ECTO} subunits (Binley et al., 2000a; Sanders et al., 2000). Properly positioned, this intermolecular disulfide bond forms efficiently during envelope glycoprotein (Env) synthesis, allowing the secretion of gp140 proteins that are proteolytically processed but in which the association between the gp120 and gp41ECTO subunits is maintained by the disulfide bond (SOS gp140 proteins). However, gp41-gp41 interactions are unstable in the SOS gp140 protein, and oligomeric forms of SOS gp140, which are not abundant, do not survive purification. Thus, purified SOS gp140 is a monomeric protein (Schulke et al., 2002). By contrast, gp140UNC proteins, with or without the SOS cysteine substitutions, are multimeric, implying that cleavage of the peptide bond between gp120 and gp41 destabilizes the native complex.

Despite being monomeric, the purified and unpurified forms of SOS gp140 are better antigenic structural mimics of the native, fusion-competent Env structure than are the corresponding gp120 or gp140UNC proteins. This may be because the presence and orientation of gp41_{ECTO} occludes certain non-neutralization epitopes on SOS gp140 while preserving the presentation of important neutralization sites. This explanation is consistent with immunoelectron microscopy studies of the protein. Unexpectedly, proteolytically mature, but variable-loop-deleted, SOS gp140 glycoproteins have enhanced oligomeric stability, so these molecules warrant further study for their structural and immunogenic properties.

Modified gp140 proteins which can be synthesized as soluble, cleaved HIV-1 Env trimers have recently been generated (WO 03/022869 A2; Sanders et al., 2002b). In these gp140 proteins, the gp120-gp41 interactions are stabilized by an intermolecular disulfide bond, and the gp41-gp41 interactions are stabilized by specific amino-acid substitutions in the N-terminal heptad repeat of gp41_{ECTO}, most notably at position 559. Substitutions introduced at position 559 to make the SOSI559P or SOSI559G gp140 proteins to block one or more of the conformational transitions in gp41_{ECTO} (Hunter, 1997). A model of how the substitutions might act is presented in FIG. 3. Preventing these transitions is believed to stabilize the SOS gp140 protein as a trimer (WO 03/022869 A2; Sanders et al., 2002b). It has also been demonstrated that the I559P substitution does not affect the ability of the gp120 subunits of an SOS gp140 protein to bind the CD4 receptor, and to then undergo receptor-mediated conformational changes within the gp120 subunits (WO 03/022869 A2; Sanders et al., 2002b).

The invention relates to the use of trimeric SOSIP proteins as immunogens in rabbits to elicit production of antibodies with neutralization activity against viral particles pseudotyped with envelopes from selected primary HIV-1 isolates and immortalized T cell line-adapted (TCLA) HIV-1 strains as well as against PBMC-grown HIV-1.

Initial studies on the immunogenicity of disulfide-stabilized, cleaved forms of the trimeric HIV-1 Env complex (SOSIP.R6 gp140) in rabbits, using a DNA-prime, soluble protein boost regimen are described herein. The principal aims of the described experiments were 4-fold: first, to obtain information on the absolute immunogenicity of SOSIP.R6 gp140 trimers; second, to compare the immunogenicity of these trimers with gp120 monomers; third, to determine whether bead-immobilized SOSIP.R6 gp140 trimers were superior to soluble forms of the same protein at eliciting an antibody response in the boosting phase; and fourth, to evaluate the relative merits of soluble or membrane-associated forms of Env proteins in the DNA priming phase. Conditions were identified under which SOSIP.R6 gp140 trimers can elicit antibodies that neutralize HIV-1_{JR-FL} and certain other representative primary isolates in pseudovirus and/or whole-virus assays. The findings have been incorporated into the design of follow-on immunogenicity studies designed to directly compare SOSIP.R6 gp140 trimers and gp120 monomers.

The results showed that it was beneficial to use a DNA priming procedure, compared to immunization with subunit proteins only. The advantages provided by DNA priming related more to the rate at which antibody responses were generated after subunit boosting and, to an extent, their longevity, rather than to the magnitude and breadth of the final NAb responses. In addition, antibody responses in primed animals were somewhat more robust and less subject to decline between protein boosts. Thus, NAb titers (or even anti-gp120 binding Ab titers) were a little higher in the DNA-primed animals after completion of all the protein boosts, compared to animals that received protein only. These findings are broadly consistent with other reports on the outcome of DNA immunizations in small animals (reviewed in Estcourt et al., 2004; Giri et al., 2004).

The use of membrane-bound and soluble forms of gp140 proteins (i.e., cleaved, disulfide-stabilized proteins that contained or lacked the gp41 transmembrane domain) during the DNA-priming phase was also compared. The exact configuration(s) in which gp140 proteins are expressed in vivo after immunization with DNA is not known; indeed, the issue appears to have never been studied. It is, for example, uncertain whether the gp140 proteins are cleaved, wholly or in part after DNA immunization. Env proteins derived from primary strains of HIV-1, when expressed on the surface of env-transfected cells in vitro, are rarely fully cleaved, and they differ from native, fusion-competent trimers in their exposure of various neutralizing and non-neutralizing antibody epitopes (Abrahamyan et al., 2003; Herrera et al., 2003). Since Env cleavage may be relevant to the design of the best antigenic mimics of the native trimer, cleavage-enhancing substitutions were incorporated into some of the gp140 proteins that were expressed in the DNA priming phase (Binley et al., 2002). The R6 modification was included because Env proteins expressed on the cell surface in vitro are usually incompletely cleaved (Herrera et al., 2003; Si et al., 2003). While this modification does improve the level of Env cleavage in transiently transfected 293T cells, assessed by surface biotinylation (data not shown), the impact that it has under in vivo conditions has not been determined. Expressing membrane-bound proteins in the DNA priming phase did offer a small but measurable advantage over soluble gp140 proteins in terms of the subsequent immune responses to the soluble protein boosts. This advantage could be due to differences in the quality, or in the quantity, of the Env proteins expressed in the DNA priming phase.

A membrane-bound protein that contains a minimal cytoplasmic domain was used. Truncation of the cytoplasmic domain of gp41 is a commonly used technique to increase Env cell surface expression for antigenicity studies, by circumventing the natural down-regulatory sequences (Bultmann et al., 2001). Both the truncated form of wild type Env and the SOS mutant retain their fusion function and can be incorporated into infectious Env-pseudotyped particles generated by transfection of 293T cells (Abrahamyan et al., 2003; Binley et al., 2003). Truncation of the cytoplasmic tail does, however, reduce Env incorporation into virions generated in T-cells in vitro, and hence decreases their infectivity. Recent cross-linking experiments suggest that full length Env expressed on the surface of virions, and by implication the cell surface, is trimeric (Center et al., 2002; Richman et al., 2003). Taken together, it was considered unnecessary to further modify the SOS.R6(T) Env form by addition of the trimer-stabilizing mutation, I559P.

When different forms of gp140 proteins were compared in the protein boosting phase, the soluble proteins were found to be slightly more immunogenic than the same proteins captured onto beads to make a particulate antigen. The bead-captured antigens were evaluated because, in principle, particulate antigens offer some theoretical immunogenicity advantages over small proteins, and because the capture of Env proteins onto beads offers some benefits from the purification perspective. They may also be advantageous for the induction of cell-mediated immune responses.

Following two DNA primes and two subunit protein boosts (week 20), the anti-gp120 binding Ab titers were ˜3-fold lower in the gp140 trimer-immunized animals than in the gp120 recipients. The titer difference may reflect an inherently reduced immunogenicity of the trimers. One possibility is that non-neutralizing epitopes are occluded within the trimer but exposed on the monomer. Another possibility is that the non-neutralizing face of gp120 is occluded in the trimer by the gp41ECTO domain (Binley et al., 2000). Alternatively, neutralizing epitopes could be less well presented on the trimer than on the monomer, because of epitope occlusion within the quaternary structure of the multimeric protein. This seems unlikely, however, because the reduced titer of binding Abs was not mirrored by a reduction in the titers of NAb in the same sera at the same time.

One goal of the study, as initially designed, was to determine whether SOSIP.R6 gp140 trimers were superior to monomeric gp120 proteins for NAb induction. This determination necessitated a comparison between the results of a Pilot study (using gp120 monomers) and a Second-stage study, which evaluated different versions of gp140 trimers. When the initial immunizations (two DNA primes, two soluble protein boosts) were completed after 20 weeks, there was no indication that the trimers were superior to the monomers as immunogens. An ad hoc decision was therefore made to extend the immunization period in the Second-stage study beyond the 20 weeks used in the Pilot study, to see if additional protein boosts could improve the antibody response to the trimers. Consequently, no formal comparison between gp120 monomers and gp140 trimers is possible after week 20. This lacuna will be addressed in follow-up studies that have now been initiated, taking into account what has been learned from the experiments reported herein.

In the later stages of the Second-stage study, NAb to both the neutralization-sensitive TCLA strain HIV-1_{MN} and the autologous primary strain, HIV-1_{JR-FL} could be induced with more consistency than in the early weeks of the experiment. In some animals, particularly #228, #238, #240 and #241, the titers of NAbs against HIV-1JR-FL were respectable, particularly in the Env-pseudotype assay. It should be noted, however, that the general experiences of various groups is that Env-pseudotyped viruses are 3- to 10-fold more sensitive to neutralization than the corresponding primary isolate, for reasons that have yet to be fully understood (Moore and Burton, 2004). Thus, neutralization titers against pseudoviruses need to be interpreted cautiously; in the real world, vaccine-induced NAbs must counter true, infectious primary viruses. These caveats notwithstanding, certain conclusions can be drawn from the neutralization assays.

Firstly, it is possible to elicit by immunization with a trimeric form of Env, SOSIP.R6 gp140, at least in a subset of rabbits, antibodies capable of neutralizing a typically resistant HIV-1 clone, JR-FL, in a single-round Env-pseudotyped assay. In most of the animals, these NAb were only present at low titers, but in rabbit #241 the 50% and 90% neutralization titers against HIV-1_{JR-FL} reached >160 and ˜20, respectively, by the final bleed. Antibodies from this animal were even capable of neutralizing PBMC-grown HIV-1_{JR-FL} in a PBMC assay; other reports of the induction of NAb to JR-FL by Env-based vaccines have not been reported. It is not known what factors caused rabbit #241 to respond so strongly. An atypically robust response to Env was also observed in a single rabbit in a study of T-helper epitopes (Grundner et al., 2004). Little if any of the NAb to JR-FL were directed against the V3 region, but essentially all of them were removed by incubation of the sera with monomeric gp120. Hence their target is one or more gp120 epitopes outside the V3 region. Identifying this epitope(s) will be a priority for future studies as this knowledge could guide the development of new immunization strategies (Burton et al., 2004).

Even the most potent of the rabbit antisera had a limited ability to cross-neutralize heterologous strains. Moreover, the generation of NAb responses to the autologous strain required an intensive and prolonged immunization regimen. Accordingly, there is scope to improve the magnitude of NAd responses, and the consistency with which the stronger responses are elicited needs to be improved. Overcoming the paucity of T-helper epitopes on Env may be important (Grundner et al., 2004). Although Env trimers offer immunogenicity advantages over gp120 monomers (VanCott et al., 1997; Yang et al., 2001; Bower et al., 2004), it is expected that the immunogenicity of these trimers can be further enhanced. This applies both to the trimers described herein and to others whose basis is the deletion of the cleavage site between gp120 and gp41_{ECTO}. It is also necessary that different env gene sequences be compared, in case some Env proteins are more immunogenic than others. Modifications designed to increase epitope exposure must also be evaluated, as should alternative ways to present Env proteins to the immune system. All of these issues are the subject of ongoing studies.

### REFERENCES

1. U.S. Pat. No. 4,816,567, issued Mar. 28, 1989 to S. Cabilly et al.
2. U.S. Pat. No. 5,225,539, issued Jul. 6, 1993 to G. P. Winter et al.
3. U.S. Pat. No. 5,585,089, issued Dec. 16, 1996 to C. L. Queen et al.
4. U.S. Pat. No. 5,693,761, issued Dec. 2, 1997 to C. L. Queen et al.
5. U.S. Pat. No. 5,866,163, issued Feb. 2, 1999 to G. L. Myers et al.
6. U.S. Pat. No. 5,869,624, issued Feb. 9, 1999 to K. W. Hasel et al.
7. U.S. Pat. No. 6,110,161, issued Aug. 29, 2000 to I. Mathiesen et al.
8. U.S. Pat. No. 6,210,663, issued Apr. 3, 2001 to H. C. J. Ertl.
9. U.S. Pat. No. 6,262,281, issued Jul. 17, 2001 to C. S. Swindell et al.
10. U.S. Pat. No. 6,610,044, issued Aug. 26, 2003 to I. Mathiesen.
11. U.S. Pat. No. 6,710,173, issued Mar. 23, 2004 to J. M. Binley et al.
12. PCT International Application Publication No. WO 90/07861, published Jul. 26, 1990 (Protein Design Labs, inc.).
13. PCT International Application Publication No. WO 00/44410, published Aug. 3, 2000 (Stichting Biomedical Primate R et al.).
14. PCT International Application Publication No. WO 03/022869, published Mar. 20, 2003 (Progenics Pharmaceuticals, Inc. et al.).
15. Abrahamyan, L. G., Markosyan, R. M., Moore, J. R., Cohen, F. S., and Melikyan, G. P. (2003) Human immunodeficiency virus type 1 Env with an intersubunit disulfide bond engages coreceptors but requires bond reduction after engagement to induce fusion. J. Virol. 77:5829-5836.
16. Allaway, G. P., Davis-Bruno K. L., Beaudry G. A., Garcia E. B., Wong E. L., Ryder A. M., Hasel K. W., Gauduin M. C., Koup R. A., McDougal J. S, and Maddon P. J. (1995) Expression and characterization of CD4-IgG2, a novel heterotetramer which neutralizes primary HIV-1 isolates. AIDS Res. Hum. Retroviruses. 11:533-539.
17. Alving, C. R., Koulchin V., Glenn G. M. and Rao M. (1995) Liposomes as carriers of peptide antigens: induction of antibodies and cytotoxic T lymphocytes to conjugated and unconjugated peptides. Immunol. Rev. 145:5-31.
18. Berman, P. W., Riddle L., Nakamura G., Haffar O. K., Nunes W. M., Skehel P., Byrn R., Groopman J., Matthews T. and Gregory T. (1989) Expression and immunogenicity of the extracellular domain of the human immunodeficiency virus type 1 envelope glycoprotein, gp160. J. Virol. 63: 3489-98.
19. Binley, J. and Moore J.P. (1997b) HIV-cell fusion. The viral mousetrap. 387:346-348.
20. Binley, J. M., Clas B., Gettie A., Vesanen M., Montefiori D. C., Sawyer L., Booth J., Lewis M., Marx P. A., Bonhoeffer S, and Moore J.P. (2000b) Passive infusion of immune serum into simian immunodeficiency virus-infected rhesus macaques undergoing a rapid disease course has minimal effect on plasma viremia. Virology 270:237-249.
21. Binley, J. M., Klasse P. J., Cao Y., Jones I., Markowitz M., Ho D. D., and Moore J.P. (1997a). Differential regulation of the antibody responses to Gag and Env proteins of human immunodeficiency virus type 1. J. Virol. 71:2799-2809.
22. Binley, J. M., Sanders R. W., Clas B., Schulke N., Master A., Guo Y., Kajumo F., Anselma D. J., Maddon P. J., Olson W. C. and Moore J.P. (2000a) A recombinant HIV-1 envelope glycoprotein complex stabilized by an intermolecular disulfide bond between the gp120 and gp41 subunits is an antigenic mimic of the trimeric virion-associated structure. J. Virol. 74:627-643.
23. Binley, J. M., Sanders, R. W., Master A., Cayanan C. S., Wiley C. L., Schiffner L., Travis B., Kuhmann S., Burton D. R., Hu S. L., Olson W. C., and Moore J. P. (2002) Enhancing the proteolytic maturation of human immunodeficiency virus type 1 envelope glycoproteins. J. Virol. 76:2606-2616.
24. Binley, J. M., Wrin T., Korber B., Zwick M. B., Wang M., Chappey C., Stiegler G., Kunert R., Zolla-Pazner S., Katinger H., Petropoulos, C. J., and Burton, D. R. (2004) Comprehensive cross-clade neutralization analysis of a panel of anti-human immunodeficiency virus type 1 monoclonal antibodies. J. Virol. 78:13232-13252.
25. Bower, J. F., Yang X., Sodroski J. and Ross T. M. (2004) Elicitation of neutralizing antibodies with DNA vaccines expressing soluble stabilized human immunodeficiency virus type 1 envelope glycoprotein trimers conjugated to C3d. J. Virol. 78:4710-4719.
26. Buchacher, A., Predl R., Strutzenberger K., Steinfellner W., Trkola A., Purtscher M., Gruber G., Tauer C., Steindl F., Jungbauer A. et al. (1994) AIDS Res. Human Retrov. 10:359-369.
27. Bultmann, A., Muranyi W., Seed B. and Haas J. (2001) Identification of two sequences in the cytoplasmic tail of the human immunodeficiency virus type 1 envelope glycoprotein that inhibit cell surface expression. J. Virol. 75:5263-5276.
28. Burton, D. R. (1997) A vaccine for HIV type 1: the antibody perspective. Proc. Natl. Acad. Sci. USA 94:10018-10023.
29. Burton, D. R. and Montefiori D. C., (1997) The antibody response in HIV-1 infection. AIDS. 11 Suppl A: S87-S98.
30. Burton, D. R. and Parren, P. H. W. I. (2000) Vaccines and the induction of functional antibodies: time to look beyond the molecules of natural infection? Nat. Med. 6:123-125.
31. Burton, D. R., Barbas C. F., Persson M. A., Koenig S., Chanock R. M., and Lerner R. A.(1991) A large array of human monoclonal antibodies to type 1 human immunodeficiency virus from combinatorial libraries of asymptomatic seropositive individuals. Proc. Nat. Acad. Sci. U.S.A. 88:10134-10137.
32. Burton, D. R., Desrosiers R. C., Doms R. W., Koff W. C., Kwong P. D., Moore J. P., Nabel G. J., Sodroski J., Wilson I. A. and Wyatt R. T. (2004) HIV vaccine design and the neutralizing antibody problem. Nat. Immunol. 5:233-236.
33. Burton, D. R., Pyati J., Koduri R., Sharp S. J., Thornton G. B, Parren P. W., Sawyer L. S., Hendry R. M., Dunlop N., Nara P. L., et al. (1994) Efficient neutralization of primary isolates of HIV-1 by a recombinant human monoclonal antibody. Science. 266:1024-1027.
34. Cao, J., Sullivan N., Desjardin E., Parolin C., Robinson J., Wyatt R. and Sodroski J. (1997) Replication and neutralization of human immunodeficiency virus type 1 lacking the V1 and V2 variable loops of the gp120 envelope glycoprotein. J. Virol. 71: 9808-9812.
35. Center, R. J., Leapman R. D., Lebowitz J., Arthur L. O., Earl P. L. and Moss B. (2002) Oligomeric structure of the human immunodeficiency virus type 1 envelope protein on the virion surface. J. Virol. 76:7863-7867.
36. Chan, D. C. and Kim P. S. (1998) HIV entry and its inhibition. Cell 93:681-684.
37. Chan, D. C., Fass D., Berger J. M. and Kim P. S. (1997) Core structure of gp41 from the HIV envelope glycoprotein. Cell 89:263-273.
38. Chen, B., Zhou G., Kim M., Chishti Y., Hussey R. E., Ely B., Skehel J. J., Reinherz E. L., Harrison S. C. and Wiley D. C. (2000) Expression, purification, and characterization of gp160e, the soluble, trimeric ectodomain of the simian immunodeficiency virus envelope glycoprotein, gp160. J. Biol. Chem. 275:34946-34953.
39. Cleland, J. L., Powell M. F., Lim A., Barron L., Berman P. W., Eastman D. J., Nunberg J. H., Wrin T., and Vennari J. C. (1994) Development of a single-shot subunit vaccine for HIV-1. AIDS Res. Hum. Retroviruses 10 Suppl 2:S21-26.
40. Cohen, J. (2003) Public health. AIDS vaccine trial produces disappointment and confusion. Science 299:1290-1291.
41. Connor, R. I., Sheridan K. E., Lai C., Zhang L. and Ho D. D. (1996) Characterization of the functional properties of env genes from long-term survivors of human immunodeficiency virus type 1 infection. J. Virol. 70:5306-5311.
42. Coombes, A. G., Lavelle E. C., and David S. S. (1999) Biodegradable lamellar particles of poly(lactide) induce sustained immune responses to a single dose of adsorbed protein. Vaccine. 2410-2422.
43. Coombes, A. G., Lavelle E. C., Jenkins P. G., and Davis S. S. (1996) Single dose, polymeric, microparticle-based vaccines: the influence of formulation conditions on the magnitude and duration of the immune response to a protein antigen. Vaccine 14:1429-1438.
44. Desrosiers, R. C. (2004) Prospects for an AIDS vaccine. Nat. Med. 10:221-223.
45. diMarzo Veronese et al. (1992) AIDS Res. Human Retrov. 8:1125.
46. Doe, B., Selby M., Barnett S., Baenziger J. and Walker C. M. (1996) Induction of cytotoxic T lymphocytes by intramuscular immunization with plasmid DNA is facilitated by bone marrow-derived cells. Proc. Natl. Acad. Sci. U.S.A. 93(16):8578-8583.
47. Doms, R. W. (2000a) Beyond receptor expression: the influence of receptor conformation, density, and affinity in HIV-1 infection. Virol. 276:229-237.
48. Doms, R. W. and Moore J. P. (2000b) HIV-1 membrane fusion: targets of opportunity. J. Cell Biol. 151:F9-14.
49. Earl P. L., Doms R. W. and Moss B. (1990) Oligomeric structure of the human immunodeficiency virus type 1 envelope glycoprotein. Proc. Nat. Acad. Sci. U.S.A. 87:648-652.
50. Earl, P. L., Moss B. and Doms R. W. (1994a) Folding, interaction with GRP78-BiP, assembly and transport of the human immunodeficiency virus type 1 envelope protein. J. Virol. 65:2047-2055.
51. Earl, P. L., Broder C. C., Doms R. W. and Moss B. (1997) Epitope map of human immunodeficiency virus type 1 gp41 derived from 47 monoclonal antibodies produced by immunization with oligomeric envelope protein. J. Virol. 71:2674-2684.
52. Earl, P. L., Broder C. C., Long D., Lee S. A., Peterson J., Chakrabarti S., Doms R. W. and Moss B. (1994b) Native oligomeric human immunodeficiency virus type 1 envelope glycoprotein elicits diverse monoclonal antibody reactivities. J. Virol. 68:3015-3026.
53. Earl, P. L., Doms R. W. and Moss B. (1990) Oligomeric structure of the human immunodeficiency virus type 1 envelope glycoprotein. Proc. Natl. Acad. Sci. U.S.A 87:648-652.
54. Earl, P. L., Sugiura W., Montefiori D. C., Broder C. C., Lee S. A., Wild C., Lifson J. and Moss B. (2001) Immunogenicity and protective efficacy of oligomeric human immunodeficiency virus type 1 gp140. J. Virol. 75:645-653.
55. Edinger, A. L., Ahuja M., Sung T., Baxter K. C., Haggarty B., Doms R. W. and Hoxie J. A. (2000) Characterization and epitope mapping of neutralizing monoclonal antibodies produced by immunization with oligomeric simian immunodeficiency virus envelope protein. J. Virol. 74:7922-7935.
56. Estcourt, M. J., McMichael A. J. and Hanke T. (2004) DNA vaccines against human immunodeficiency virus type 1. Immunol. Rev. 199:144-155.
57. Farzan, M., Choe H., Desjardins E., Sun Y., Kuhn J., Cao J., Archambault D., Kolchinsky P., Koch M., Wyatt R., and Sodroski J. (1998) Stabilization of human immunodeficiency virus type 1 envelope glycoprotein trimers by disulfide bonds introduced into the gp41 glycoprotein ectodomain. J. Virol. 72:7620-7625.
58. Feltquate, D. M., Heaney S., Webster R. G. and Robinson H. L. (1997) Different T helper cell types and antibody isotypes generated by saline and gene gun DNA immunization. J. Immunol. 158(5):2278-2284.
59. Fouts, T. R., Binley J. M., Trkola A., Robinson J. E. and Moore J.P. (1997) Neutralization of the human immunodeficiency virus type 1 primary isolate JR-FL by human monoclonal antibodies correlates with antibody binding to the oligomeric form of the envelope glycoprotein complex. J Virol 71: 2779-85.
60. Fouts, T. R., Trkola A., Fung M. S. and Moore J. P. (1998) Interactions of polyclonal and monoclonal anti-glycoprotein 120 antibodies with oligomeric glycoprotein 120-glycoprotein 41 complexes of a primary HIV type 1 isolate: relationship to neutralization. AIDS Res. Hum. Retrovir. 14:591-597.
61. Garber, D. A., Silvestri G. and Feinberg M. B. (2004) Prospects for an AIDS vaccine: three big questions, no easy answers. Lancet Infect. Dis. 4:397-413.
62. Gerl, M., Jaenicke R., Smith J. M. and Harrison P.M. (1988) Self-assembly of apoferritin from horse spleen after reversible chemical modification with 2,3-dimethylmaleic anhydride. Biochemistry 27:4089-4096.
63. Gordon, C. J., Muesing M. A., Proudfoot A. E., Power C. A., Moore J. P. and Trkola, A. (1999) Enhancement of human immunodeficiency virus type 1 infection by the CC-chemokine RANTES is independent of the mechanism of virus-cell fusion. J. Virol. 73:684-694.
64. Gorse, G. J., McElrath M. J., Matthews T. J., Hsieh R. H., Belshe R. B., Corey L., Frey S. E., Kennedy D. J., Walker M. C. and Eibl M. M. (1998) Modulation of immunologic responses to HIV-1MN recombinant gp160 vaccine by dose and schedule of administration. National Institute of Allergy and Infectious Diseases AIDS Vaccine Evaluation Group. Vaccine 16:493-506.
65. Graham, B. S. (2002) Clinical trials of HIV vaccines. Annu. Rev. Med. 53:207-221.
66. Grundner, C., Pancera M., Kang J. M., Koch M., Sodroski J. and Wyatt, R. (2004) Factors limiting the immunogenicity of HIV-1 gp120 envelope glycoproteins. Virology 330:233-248.
67. Hanes, J., Cleland J. L. and Langer R. (1997) New advances in microsphere-based single-dose vaccines. Advanced Drug Delivery Reviews 28:97-119.
68. He, Q., Mitchell A. R., Johnson S. L., Wagner-Bartak C., Morcol T. and Bell S. J. (2000) Calcium phosphate nanoparticle adjuvant. Clinical and Diagnostic Laboratory Immunology 7:899-903.
69. Herrera, C., Spenlehauer C., Fung M. S., Burton D. R., Beddows S. and Moore J. P. (2003) Nonneutralizing antibodies to the CD4-binding site on the gp120 subunit of human immunodeficiency virus type 1 do not interfere with the activity of a neutralizing antibody against the same site. J. Virol. 77:1084-1091.
70. Hoffman, T. L., Canziani G., Jia L., Rucker J. and Doms R. W. (2000) A biosensor assay for studying ligand-membrane receptor interactions: binding of antibodies and HIV-1 Env to chemokine receptors. Proc. Natl. Acad. Sci. U.S.A. 97:11215-11220.
71. Hu, S. L., Klaniecki J., Dykers T., Sridhar P. and Travis B. M. (1991) Neutralizing antibodies against HIV-1 BRU and SF2 isolates generated in mice immunized with recombinant vaccinia virus expressing HIV-1 (BRU) envelope glycoproteins and boosted with homologous gp160. AIDS Res. Hum. Retroviruses 7:615-620.
72. Hunter, E. (1997) gp41, a multifunctional protein involved in HIV entry and pathogenesis, p. III-55-III-73. In B. Korber, B. Hahn, B. Foley, J. W. Mellors, T. Leitner, G. Myers, F. McCutchan, and C. L. Kuiken (eds.), Human Retroviruses and AIDS 1997. Theoretical Biology and Biophysics Group, Los Alamos National Laboratory, Los Alamos, N. Mex.
73. Johnson, W. E. and Desrosiers R. C. (2002) Viral persistence: HIV's strategies of immune system evasion. Annu. Rev. Med. 53:499-518.
74. Jones, P. L., Korte T. and Blumenthal R. (1998) Conformational changes in cell surface HIV-1 envelope glycoproteins are triggered by cooperation between cell surface CD4 and co-receptors. J. Biol. Chem. 273:404-409.
75. Kovacsovics-Bankowski, M. and Rock K. L. (1995) A phagosome-to-cytosol pathway for exogenous antigens presented on MHC class I molecules. Science 267:243-246.
76. Kwong, P. D., Wyatt R., Robinson J., Sweet R. W., Sodroski J. and Hendrickson W. A. (1998) Structure of an HIV gp120 envelope glycoprotein in a complex with the CD4 receptor and a neutralizing human antibody. Nature 393:648-659.
77. Kwong, P. D., Wyatt R., Sattentau Q. J., Sodroski J. and Hendrickson W. A. (2000) Oligomeric modeling and electrostatic analysis of the gp120 envelope glycoprotein of human immunodeficiency virus. J. Virol. 74:1961-1972.
78. Langhein, C. and Newman J. F. (1987) Antibody response to bacterial antigens covalently bound to biodegradable polymerized serum albumin beads. Journal of Applied Bacteriology 63:443-448.
79. Lu, M., Ji H. and Shen S. (1999) Subdomain folding and biological activity of the core structure from human immunodeficiency virus type 1 gp41: implications for viral membrane fusion. J. Virol. 73:4433-4438.
80. Lu, M., S. Blackow and P. Kim. 1995. A trimeric structural domain of the HIV-1 transmembrane glycoprotein. Nat. Struct. Biol. 2:1075-1085.
81. Lu, M., Stoller M. O., Wang S., Liu J., Fagan M. B. and Nunberg J. H. (2001) Structural and Functional Analysis of Interhelical Interactions in the Human Immunodeficiency Virus Type 1 gp41 Envelope Glycoprotein by Alanine-Scanning Mutagenesis. J. Virol. 75:11146-11156.
82. Lu, S., Wyatt R., Richmond J. F., Mustafa F., Wang S., Weng J., Montefiori D. C., Sodroski J. and Robinson H. L. (1998) Immunogenicity of DNA vaccines expressing human immunodeficiency virus type 1 envelope glycoprotein with and without deletions in the V1/2 and V3 regions. AIDS Res. Hum. Retrovir. 14:151-155.
83. Ly, A. and Stamatatos L. (2000) V2 loop glycosylation of the human immunodeficiency virus type 1 SF162 envelope facilitates interaction of this protein with CD4 and CCR5 receptors and protects the virus from neutralization by anti-V3 loop and anti-CD4 binding site antibodies. J. Virol. 74:6769-76.
84. Mascola, J. R., Snyder S. W., Weislow O. S., Belay S. M., Belshe R. B., Schwartz D. H., Clements M. L., Dolin R., Graham B. S, Gorse G. J., Keefer M. C., McElrath M. J., Walker M. C., Wagner K. F., McNeil J. G., McCutchan F. E. and Burke D.S. (1996) Immunization with envelope subunit vaccine products elicits neutralizing antibodies against laboratory-adapted but not primary isolates of human immunodeficiency virus type 1. The National Institute of Allergy and Infectious Diseases AIDS Vaccine Evaluation Group. Infec. Dis. 173:340-348.
85. McMichael, A. J. and Hanke T. (2003) HIV vaccines 1983-2003. Nat. Med. 9:874-880.
86. Melikyan, G. B., Markosyan R. M., Hemmati H., Delmedico M. K., Lambert D. M. and Cohen F. S. (2000) Evidence that the transition of HIV-1 gp41 into a 6-helix bundle, not the bundle configuration, induces membrane fusion. J. Cell Biol. 151:413-423.
87. Men, Y., Gander B., Merkle H. P. and Corradin G. (1996) Induction of sustained and elevated immune responses to weakly immunogenic synthetic malarial peptides by encapsulation in biodegradable polymer microspheres. Vaccine 14:1442-1450.
88. Moore, J. P. and Binley J. (1998) HIV. Envelope's letters boxed into shape. Nature 393:630-631.
89. Moore, J. P. and Burton, D. R. (2004) Urgently needed: a filter for the HIV-1 vaccine pipeline. Nat. Med. 10:769-771.
90. Moore, J. P. and Sodroski J. (1996) Antibody cross-competition analysis of the human immunodeficiency virus type 1 gp120 exterior envelope glycoprotein. J. Virol. 70:1863-1872.
91. Moore, J. P. and Stevenson M. (2000) New targets for inhibitors of HIV-1 replication. Nat. Rev. Mol. Cell Biol. 1:40-49.
92. Moore, J. P. and Ho D. D. (1995b) HIV-1 neutralization: the consequences of viral adaptation to growth on transformed T cells. AIDS 9 (Suppl. A):S117-S136.
93. Moore, J. P., Cao Y., Leu J., Qin L., Korber B. and Ho D. D. (1996) Inter- and intraclade neutralization of human immunodeficiency virus type 1: genetic clades do not correspond to neutralization serotypes but partially correspond to gp120 antigenic serotypes. J. Virol. 70:427-444.
94. Moore, J. P., McKeating J. A., Weiss R. A. and Sattentau Q. J. (1990) Dissociation of gp120 from HIV-1 virions induced by soluble CD4. Science 250:1139-1142.
95. Moore, J. P., Parren P. W. H. I. and Burton D. R. (2001) Genetic Subtypes, Humoral Immunity, and Human Immunodeficiency Virus Type 1 Vaccine Development. J. Virol. 75:5721-5729.
96. Moore, J. P., Sattentau Q. J., Wyatt R. and Sodroski J. (1994a) Probing the structure of the human immunodeficiency virus surface glycoprotein gp120 with a panel of monoclonal antibodies. J. Virol 68:469-484.
97. Moore, J. P., Trkola A., Korber B., Boots L. J., Kessler J. A. 2nd, McCutchan F. E., Mascola J., Ho D. D., Robinson J. and Conley A. J. (1995) HIV-1 neutralization: the consequences of viral adaptation to growth on transformed T cells. AIDS. 9 [Suppl A]: S117-S136.
98. Moore, J. P., Trkola A., Korber B., Boots L. J., Kessler J. A. 2nd, McCutchan F. E., Mascola J., Ho D. D., Robinson J. and Conley A. J. (1995a) A human monoclonal antibody to a complex epitope in the V3 region of gp120 of human immunodeficiency virus type 1 has broad reactivity within and outside clade B. J. Virol. 69:122-130.
99. Moore, J. P., Willey R. L., Lewis G. K., Robinson J. and Sodroski J. (1994b) Immunological evidence for interactions between the first, second, and fifth conserved domains of the gp120 surface glycoprotein of human immunodeficiency virus type 1. J. Virol. 68:6836-6847.
100. Muster, T., Guinea R., Trkola A., Purtscher M., Klima A., Steindl F., Palese P. and Katinger H. (1994) Cross-neutralizing activity against divergent human immunodeficiency virus type 1 isolates induced by the gp41 sequence ELDKWAS. J. Virol. 68:4031-4034.
101. Muster, T., Steindl F., Purtscher M., Trkola A., Klima A., Himmler G., Ruker F. and Katinger H. (1993) A conserved neutralizing epitope on gp41 of human immunodeficiency virus type 1. J. Virol. 67:6642-6647.
102. O'Brien, W. A., Koyanagi, Namazie A., Zhao J. Q., Diagne A., Idler K., Zack J. A. and Chen I. S. (1990) HIV-1 tropism for mononuclear phagocytes can be determined by regions of gp120 outside the CD4-binding domain. Nature 348:69-73.
103. O'Hagan, D. T., Jeffery H. and Davis S. S. (1993) Long-term antibody responses in mice following subcutaneous immunization with ovalbumin entrapped in biodegradable microparticles. Vaccine 11:965-969.
104. Okayama, H. and Berg P. (1983) A cDNA cloning vector that permits expression of cDNA inserts in mammalian cells. Mol. Cell. Biol. 3(2):280-289.
105. Parker, C. E., Deterding L. J., Hager-Braun C., Binley J. M., Schulke N., Katinger H., Moore J.P. and Tomer K. B. (2001) Fine definition of the epitope on the gp41 glycoprotein of human immunodeficiency virus type 1 for the neutralizing monoclonal antibody 2F5. J. Virol. 75:10906-10911.
106. Parren, P. W., Burton D. R. and Sattentau Q. J. (1997a) HIV-1 antibody—debris or virion? Nat. Med. 3:366-367.
107. Parren, P. W., Fisicaro P., Labrijn A. F., Binley J. M., Yang W. P., Ditzel H. J., Barbas C. F. 3^{rd} and Burton D. R. (1996) In vitro antigen challenge of human antibody libraries for vaccine evaluation: the human immunodeficiency virus type 1 envelope. J. Virol. 70:9046-9050.
108. Parren, P. W., Gauduin, M. C., Koup, R. A., Poignard, P., Sattentau, W. J., Fisicaro, P. and Burton, D. R. (1997b) Relevance of the antibody response against human immunodeficiency virus type 1 envelope to vaccine design. Immunol. Lett. 58:125-132.
109. Parren, P. W., Mondor I., Naniche D., Ditzel H. J., Klasse P. J., Burton D. R. and Sattentau Q. J. (1998) Neutralization of human immunodeficiency virus type 1 by antibody to gp120 is determined primarily by occupancy of sites on the virion irrespective of epitope specificity. J. Virol. 72:3512-3519.
110. Parren, P. W. H. I. and Burton D. R. (2001) The antiviral activity of antibodies in vitro and in vivo. Adv. Immunol. 77:195-262.
111. Parren, P. W. H. I., Moore J. P., Burton D. R. and Sattentau Q. J. (1999) The neutralizing antibody response to HIV-1: viral evasion and escape from humoral immunity. AIDS 13 (Suppl. A):S137-S162.
112. Partidos, C. D., Vohra P., Anagnostopoulou C., Jones D. H., Farrar G. H. and Steward M. W. (1996) Biodegradable microparticles as a delivery system for measles virus cytotoxic T cell epitopes. Mol. Immunol. 33:485-491.
113. Pertmer, T. M., Roberts T. R. and Haynes J. R. (1996) Influenza virus nucleoprotein-specific immunoglobulin G subclass and cytokine responses elicited by DNA vaccination are dependent on the route of vector DNA delivery. J. Virol. 70(9):6119-6125.
114. Poignard, P., Saphire E. O., Parren P. W. and Burton D. R. (2001) gp120: Biologic aspects of structural features. Annu. Rev. Immunol. 19:253-274.
115. Powell, M. F., Cleland J. L., Eastman D. J., Lim A., Murthy K., Newman M. J., Nunberg J. H., Weissburg R. P., Vennari J.C. and Wrin T. (1994) Immunogenicity and HIV-1 virus neutralization of MN recombinant glycoprotein 120/HIV-1 QS21 vaccine in baboons [published erratum appears in AIDS Res Hum Retroviruses 1995 11(5):661]. AIDS Res. Hum. Retroviruses 10 Suppl 2:S105-8:S105-S108.
116. Prayaga, S. K., Ford M. J. and Haynes J. R. (1997) Manipulation of HIV-1 gp120-specific immune responses elicited via gene gun-based DNA immunization. Vaccine. 15(12-13):1349-52.
117. Raychaudhuri, S, and Rock K. L. (1998) Fully mobilizing host defense: building better vaccines. Nat. Biotechnol. 16:1025-1031.
118. Raz, E., Tighe H., Sato Y., Corr M., Dudler J. A., Roman M., Swain S. L., Spiegelberg H. L. and Carson D. A. (1996) Preferential induction of a Th1 immune response and inhibition of specific IgE antibody formation by plasmid DNA immunization. Proc. Natl. Acad. Sci. U.S.A. 93(10):5141-5145.
119. Reitter, J. N., Means R. E. and Desrosiers R. C. (1998) A role for carbohydrates in immune evasion in AIDS. Nat. Med. 4:679-684.
120. Richardson, T. M., Jr., Stryjewski B. L., Broder C. C., Hoxie J. A., Mascola J. R., Earl P. L. and Doms R. W. (1996) Humoral response to oligomeric human immunodeficiency virus type 1 envelope protein. J. Virol. 70:753-62.
121. Richman, D. D., Wrin T., Little S. J. and Petropoulos C. J. (2003) Rapid evolution of the neutralizing antibody response to HIV type 1 infection. Proc. Natl. Acad. Sci. USA 100:4144-4149.
122. Robinson, H. L. (1997) Nucleic acid vaccines: an overview. Vaccine. 15(8):785-787.
123. Rock, K. L. and Clark K. (1996) Analysis of the role of MHC class II presentation in the stimulation of cytotoxic T lymphocytes by antigens targeted into the exogenous antigen-MHC class I presentation pathway. J. Immunol. 156:3721-3726.
124. Sanders, R. W., Schiffner L., Master A., Kajumo F., Guo Y., Dragic T., Moore J. P. and Binley J. M. (2000) Variable-loop-deleted variants of the human immunodeficiency virus type 1 envelope glycoprotein can be stabilized by an intermolecular disulfide bond between the gp120 and gp41 subunits. J. Virol. 74:5091-5100.
125. Sanders, R. W., Venturi M., Schiffner L., Kalyanaraman R., Katinger H., Kwong P. D. and Moore J.P. (2002a) The mannose-dependent epitope for the neutralizing antibody 2G12 on the gp120 glycoprotein of human immunodeficiency virus type 1. J. Virol. 76:7293-7305.
126. Sanders, R. W., Vesanen M., Schuelke N., Master A., Schiffner L., Kalyanaraman R., Paluch M., Berkhout B., Maddon P. J., Olson W. C., Lu M. and Moore J.P. (2002b) Stabilization of the soluble, cleaved, trimeric form of the envelope glycoprotein complex of human immunodeficiency virus type 1. J. Virol. 76:8875-8889.
127. Sattentau, Q. J. and Moore J. P. (1991) Conformational changes induced in the human immunodeficiency virus envelope glycoprotein by soluble CD4 binding. J. Exp. Med. 174:407-415.
128. Sattentau, Q. J. and Moore J.P. (1995a) Human immunodeficiency virus type 1 neutralization is determined by epitope exposure on the gp120 oligomer. J. Exp. Med. 182:185-196.
129. Sattentau, Q. J., Zolla-Pazner S, and Poignard P. (1995b) Epitope exposure on functional, oligomeric HIV-1 gp41 molecules. Virology 206:713-717.
130. Scandella, C. J., Kilpatrick J., Lidster W., Parker C., Moore J. P., Moore G. K., Mann K. A., Brown P., Coates S., Chapman B., et al. (1993) Nonaffinity purification of recombinant gp120 for use in AIDS vaccine development. AIDS Res. Hum. Retroviruses 9:1233-1244.
131. Schagger, H., Cramer W. A. and von Jagow G. (1994) Analysis of molecular masses and oligomeric states of protein complexes by blue native electrophoresis and isolation of membrane protein complexes by two-dimensional native electrophoresis. Analytical Biochemistry 217:220-230.
132. Schulke, N., Vesanen M. S., Sanders R. W., Zhu P., Anselma D. J., Villa A. R., Parren P. W. H. I., Binley J. M., Roux K. H., Maddon P. J., Moore J. P. and Olson W. C. (2002) Oligomeric and conformational properties of a proteolytically mature, disulfide-stabilized human immunodeficiency virus type 1 gp140 envelope glycoprotein. J. Virol. 76:7760-7776.
133. Si Z., Phan N., Kiprilov E. and Sodroski J. (2003) Effects of HIV type 1 envelope glycoprotein proteolytic processing on antigenicity. AIDS Res Hum Retroviruses 19:217-226.
134. Sjolander, A., van't Land B. and Lovgren Bengtsson K. (1997). Iscoms containing purified Quillaja saponins upregulate both Th1-like and Th2-like immune responses. Cell. Immunol. 177(1):69-76.
135. St. Clair, N., Shenoy B., Jacob L. D. and Margolin A. L. (1999) Cross-linked protein crystals for vaccine delivery. Proc. Nat. Acad. Sci. U.S.A. 96:9469-9474.
136. Stamatatos, L., Lim M. and Cheng-Mayer C. (2000) Generation and structural analysis of soluble oligomeric gp140 envelope proteins derived from neutralization-resistant and neutralization-susceptible primary HIV type 1 isolates. AIDS Res. Hum. Retrovir. 16:981-994.
137. Sullivan, N., Sun Y., Sattentau Q., Thali M., Wu D., Denisova G., Gershoni J., Robinson J., Moore J. and Sodroski J. (1998) CD4-Induced conformational changes in the human immunodeficiency virus type 1 gp120 glycoprotein: consequences for virus entry and neutralization. J. Virol. 72:4694-4703.
138. Tan, K., Liu J., Wang J., Shen S, and Lu M. (1997) Atomic structure of a thermostable subdomain of HIV-1 gp41. Proc. Natl. Acad. Sci. U.S.A 94:12303-12308.
139. Thali, M., Moore J. P., Furman C., Charles M., Ho D. D., Robinson J. and Sodroski J. (1993) Characterization of conserved human immunodeficiency virus type 1 gp120 neutralization epitopes exposed upon gp120-CD4 binding. J. Virol. 67:3978-3988.
140. Thomas, G., Thorne B. A., Thomas L., Allen R. G., Hruby D. E., Fuller R. and Thorner J. (1988) Yeast KEX2 endopeptidase correctly cleaves a neuroendocrine prohormone in mammalian cells. Science 241:226-230.
141. Trkola, A., Dragic T., Arthos J., Binley J. M., Olson W. C., Allaway G. P., Cheng-Mayer C., Robinson J., Maddon P. J. and Moore J. P. (1996a) CD4-dependent, antibody-sensitive interactions between HIV-1 and its co-receptor CCR-5. Nature 384:184-187.
142. Trkola, A., Ketas T., Kewalramani V. N., Endorf F., Binley J. M., Katinger H., Robinson J., Littman D. R. and Moore J.P. (1998) Neutralization sensitivity of human immunodeficiency virus type 1 primary isolates to antibodies and CD4-based reagents is independent of coreceptor usage. J. Virol. 72:1876-1885.
143. Trkola, A., Pomales A. B., Yuan H., Korber B., Maddon P. J., Allaway G. P., Katinger H., Barbas C. F. 3rd, Burton D. R., Ho D. D., et al. (1995) Cross-clade neutralization of primary isolates of human immunodeficiency virus type 1 by human monoclonal antibodies and tetrameric CD4-IgG. J. Virol. 69:6609-6617.
144. Trkola, A., Purtscher M., Muster T., Ballaun C., Buchacher A., Sullivan N., Srinivasan K., Sodroski J., Moore J.P. and Katinger H. (1996b) Human monoclonal antibody 2G12 defines a distinctive neutralization epitope on the gp120 glycoprotein of human immunodeficiency virus type 1. J. Virol. 70:1100-1108.
145. VanCott, T. C., Mascola J. R., Kaminski R. W., Kalyanaraman V., Hallberg P. L., Burnett P. R., Ulrich J. T., Rechtman D. J. and Birx D. L. (1997) Antibodies with specificity to native gp120 and neutralization activity against primary human immunodeficiency virus type 1 isolates elicited by immunization with oligomeric gp160. J. Virol. 71:4319-4330.
146. Vidard, L., Kovacsovics-Bankowski M., Kraeft S. K., Chen L. B., Benacerraf B. and Rock K. L. (1996) Analysis of MHC class II presentation of particulate antigens of B lymphocytes. J. Immunol. 156:2809-2818.
147. Weissenhorn, W., Dessen A., Harrison S. C., Skehel J. J. and Wiley D. C. (1997) Atomic structure of the ectodomain from HIV-1 gp41. Nature 387:426-430.
148. White-Scharf, M. E., Potts B. J., Smith L. M., Sokolowski K. A., Rusche J. R. and Silver S. (1993) Broadly neutralizing monoclonal antibodies to the V3 region of HIV-1 can be elicited by peptide immunization. Virology 192:197-206.
149. Wu, L., Gerard N. P., Wyatt R., Choe H., Parolin C., Ruffing N., Borsetti A., Cardoso A. A., Desjardin E., Newman W., Gerard C. and Sodroski J. (1996) CD4-induced interaction of primary HIV-1 gp120 glycoproteins with the chemokine receptor CCR-5. Nature 384:179-183.
150. Wyatt, R. and Sodroski J. (1998) The HIV-1 envelope glycoproteins: fusogens, antigens and immunogens. Science 280:1884-1888.
151. Wyatt, R., Kwong P. D., Desjardins E., Sweet R. W., Robinson J., Hendrickson W. A. and Sodroski J. G. (1998) The antigenic structure of the HIV gp120 envelope glycoprotein. Nature 393:705-711.
152. Xu, J. Y., Gorny M. K., Palker T., Karwowska S, and Zolla-Pazner S. (1991) Epitope mapping of two immunodominant domains of gp41, the transmembrane protein of human immunodeficiency virus type 1, using ten human monoclonal antibodies. J. Virol. 65:4832-4838.
153. Yang, X., Farzan M., Wyatt R. and Sodroski J. (2000b) Characterization of stable, soluble trimers containing complete ectodomains of human immunodeficiency virus type 1 envelope glycoproteins. J. Virol. 74:5716-5725.
154. Yang, X., Florin L., Farzan M., Kolchinsky P., Kwong P. D., Sodroski J. and Wyatt R. (2000a) Modifications that stabilize human immunodeficiency virus envelope glycoprotein trimers in solution. J. Virol. 74:4746-4754.
155. Yang, X., Wyatt R. and Sodroski J. (2001) Improved elicitation of neutralizing antibodies against primary human immunodeficiency viruses by soluble stabilized envelope glycoprotein trimers. J. Virol. 75:1165-1171.
156. Ye, L., Bu Z., Vzorov A., Taylor D., Compans R. W. and Yang C. (2004) Surface stability and immunogenicity of the human immunodeficiency virus envelope glycoprotein: role of the cytoplasmic domain. J. Virol. 78:13409-13419
157. Zhang, C. W., Chishti Y., Hussey R. E. and Reinherz E. L. (2001) Expression, purification, and characterization of recombinant HIV gp140. The gp41 ectodomain of HIV or simian immunodeficiency virus is sufficient to maintain the retroviral envelope glycoprotein as a trimer. J. Biol. Chem. 276:39577-39585.
158. Zhang, W., Canziani G., Plugariu C., Wyatt R., Sodroski J., Sweet R., Kwong P., Hendrickson W. and Chaiken I. (1999) Conformational changes of gp120 in epitopes near the CCR5 binding site are induced by CD4 and a CD4 miniprotein mimetic. Biochemistry 38:9405-9416.
159. Zwick, M. B., Labrijn A. F., Wang M., Spenlehauer C., Saphire E. O., Binley J. M., Moore J. P., Stiegler G., Katinger H., Burton D. R. and Parren P. W. (2001) Broadly neutralizing antibodies targeted to the membrane-proximal external region of human immunodeficiency virus type 1 glycoprotein gp41. J. Virol. 75:10892-10905.

1. A method for eliciting an immune response in a subject comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a trimeric protein complex, wherein each monomeric polypeptide unit in the complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

2. The method of claim 1, wherein the gp41 comprises an I559P substitution.

3. The method of claim 1, wherein the modified gp120 comprises an A501C mutation.

4. The method of claim 1, wherein the modified gp91 comprises a T605C mutation.

5. (canceled)

6. The method of claim 1, wherein the protein boost further comprises a pharmaceutically acceptable carrier.

7-22. (canceled)

23. The method of claim 1, wherein the nucleic acid prime is a DNA prime.

24-28. (canceled)

29. The method of claim 1, wherein the DNA prime encodes a variant of HIV-I gp140 comprising a gp41.

30. (canceled)

31. The method of claim 1, wherein the codons in the DNA prime have been optimized for expression in mammalian cells.

32. The method of claim 31, wherein codon optimization increases the number of CpG motifs in the DNA prime.

33-37. (canceled)

38. The method of claim 1, further comprising administering al least one adjuvant in combination with the protein boost composition.

39. The method of claim 38, wherein the at least one adjuvant is a saponin or a monophosphoryl lipid A (MPL) adjuvant.

40. (canceled)

41. The method of claim 38, wherein a first and a second adjuvant are administered sequentially in multiple boosts.

42-43. (canceled)

44. A method for preventing a subject from becoming infected with HIV-I comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-I.

45. (canceled)

46. A method for reducing the likelihood of a subject becoming infected with HIV-I comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a trimeric protein complex, wherein each monomeric polypeptide unit in the complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-I.

47. (canceled)

48. A method for preventing or delaying the onset of, or slowing the rate of progression of, an HIV-1-related disease in an HIV-1-infected subject which comprises administering to the subject as part of a regimen (i) more than one dose of a nucleic acid prime; and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a trimeric protein complex, wherein each monomeric polypeptide unit in the complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

49. A method for eliciting an immune response in a subject comprising administering to the subject as part of a regimen (i) more than one dose of a nucleic acid prime, and (ii). more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically or therapeutically effective dose of a superparamagnetic particle and a trimeric protein complex so affixed thereto as to permit recognition of the complex by a subject's immune system, wherein in the trimeric complex each monomeric polypeptide unit comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41.

50. The method of claim 49, wherein the trimeric protein complex is affixed to the superparamagnetic particle monovalently via one of three available epitopes in the complex, so as to leave two epitopes accessible to permit recognition of the complex by a subject's immune system.

51-92. (canceled)

93. A method for preventing a subject from becoming infected with HIV-I comprising administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent the subject from becoming infected with HIV-I.

94. (canceled)

95. A method for reducing the likelihood of a subject becoming infected with HIV-I comprising administering to the subject as part of a regimen (i) more than one dose of a nucleic acid prime, and (u) more than one dose of a protein boost composition, wherein each dose of the nucleic acid prime is administered to the subject at a first predefined interval and each dose of the protein boost composition is administered to the subject at a second predefined interval, and wherein the protein boost composition comprises a prophylactically effective dose of a composition comprising a superparamagnetic particle and a trimeric protein complex so affixed thereto as to permit recognition of the complex by the subject's immune system, wherein each monomeric polypeptide unit in the trimeric complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby reduce the likelihood of the subject becoming infected with HIV-I.

96. (canceled)

97. A method for preventing or delaying the onset of, or slowing the rate of progression of, an HIV-1-related disease in an HIV-1-infected subject which comprises administering to the subject as part of a regimen (i) more than one dose of a nucleic acid prime, and (ii) more than one dose of a protein boost composition, wherein each dose of the nucleic acid prime is administered to the subject at a first predefined interval and each dose of the protein boost composition is administered to the subject at a second predefined interval, and wherein the protein boost composition comprises a therapeutically effective dose of a composition comprising a superparamagnetic particle and a trirαeric protein complex so affixed thereto as to permit recognition of the complex by the subject's immune system, wherein each monomeric polypeptide unit in the trimeric complex comprises a modified HIV-I gp120 and a modified HIV-I gp41, wherein in each monomeric unit the modified gp41 has at least one amino acid substitution in its N-terminal helix and the modified gp120 and the modified gp41 are bound to each other by at least one disulfide bond between a cysteine residue substituted for a residue normally present in the amino acid sequence of the gp120 and a cysteine residue substituted for a residue normally present in the amino acid sequence of the gp41, so as to thereby prevent or delay the onset of, or slow the rate of progression of, the HIV-1-related disease in the subject.

98-105. (canceled)
